(19) United States
(12) Patent Application Publication
(10) Pub. No.: US 2023/0389583 A1
(43) Pub. Date: Dec. 7, 2023

(54) FOOD INGREDIENT

(71) Applicant: DUPONT NUTRITION BIOSCIENCES APS, Kongens Lyngby (DK)

(72) Inventors: Pieter Hendrik VAN DER ZAAL, Kongens Lyngby (DK); Marianne TOFTDAL, Kongens Lyngby (DK); Line GULDBRAND, Kongens Lyngby (DK); Kim Andkjaer HARRIT, Kongens Lyngby (DK); Goker BABACAN, Kongens Lyngby (DK)

(21) Appl. No.: 18/249,139

(22) PCT Filed: Oct. 13, 2021
(86) PCT No.: PCT/EP2021/078343

§ 371 Date: Apr. 14, 2023

(30) Foreign Application Priority Data
Oct. 14, 2020 (EP) 20201775.2
Apr. 29, 2021 (EP) 21171139.5

(51) Int. Cl. A23L 17/60; A23L 29/256; A23L 33/185

(52) U.S. Cl. CPC A23L 17/60; A23L 29/256; A23L 33/185

(57) ABSTRACT

There is provided a food ingredient obtained from seaweed of the class of Rhodophyta wherein (a) the food ingredient contains mu carrageenan in an amount of at least 4 wt. % based on the total weight of the food ingredient; and (b) the weight average molecular weight of carrageenan present in the food ingredient is at least 700 kDa.

### FIELD OF THE INVENTION

The present invention relates to a food ingredient, a composition comprising the food ingredient, a foodstuff containing the food ingredient or the composition and to methods and uses utilising the food ingredient or composition.

### BACKGROUND OF THE INVENTION

There is a growing consumer interest for foods and beverages that are produced using methods, and ingredients retaining naturalness of their raw material, and absent of chemically modified components. This consumer approach has been partly responded to by the industry offering reformulated or new products, in which some food additives, particularly preservatives, colors, and flavors have been successfully eliminated, or replaced with more positively recognised alternative ingredients and food additives of natural origin.

However, providing non-chemically modified alternatives to many food and beverage applications has remained a challenge due to absence of economically feasible methods to provide new products to deliver unique and desired functionalities (e.g. viscosity improvements and stabilisation in beverages) without negatively affecting the sensory properties (e.g. color, odor, taste) of the food or beverage system. Furthermore, these non-chemically modified new products need to be positively recognised by consumers and regulatory as discrete entities, and not chemically modified ingredients when compared to existing additives.

It would thus be desirable to provide a food ingredient which provides useful functionality, such as reduction of sedimentation in a beverage, yet which is considered by consumers and/or recognised by regulators as a natural product.

### SUMMARY OF THE INVENTION

In one aspect there is provided a food ingredient obtained from seaweed of the class of Rhodophyta wherein
(a) the food ingredient contains mu carrageenan in an amount of at least 4 wt. % based on the total weight of the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa.

In one aspect there is provided a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) a protein.

In one aspect there is provided a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) a polysaccharide selected from galactomannans, glucomannans, and mixtures thereof.

In one aspect there is provided a method for improving the stability of a foodstuff, the method comprising the step of combining a food ingredient obtained from seaweed of the class of Rhodophyta wherein
(a) the food ingredient contains mu carrageenan in an amount of at least 4 wt. % based on the total weight of the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa; and
   with the foodstuff.

In one aspect there is provided a method for improving the stability of a foodstuff, the method comprising the step of combining a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) (1) a protein or (2) a polysaccharide selected from galactomannans, glucomannans, and mixtures thereof;
   with the foodstuff.

In one aspect there is provided use of a food ingredient obtained from seaweed of the class of Rhodophyta wherein
(a) the food ingredient contains mu carrageenan in an amount of at least 4 wt. % based on the total weight of the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa; and
   for improving the stability of a foodstuff.

In one aspect there is provided use of a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) (1) a protein or (2) a polysaccharide selected from galactomannans, glucomannans, and mixtures thereof;
   for improving the stability of a foodstuff.

In one aspect there is provided a method for reducing sedimentation in a beverage, the method comprising the step of combining a food ingredient obtained from seaweed of the class of Rhodophyta wherein
(a) the food ingredient contains mu carrageenan in an amount of at least 4 wt. % based on the total weight of the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa; and
   with the foodstuff.

In one aspect there is provided a method for reducing sedimentation in a beverage, the method comprising the step of combining a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) (1) a protein or (2) a polysaccharide selected from galactomannans, glucomannans, and mixtures thereof;
   with the foodstuff.

In one aspect there is provided use of a food ingredient obtained from seaweed of the class of Rhodophyta wherein
(a) the food ingredient contains mu carrageenan in an amount of at least 4 wt. % based on the total weight of the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa; and
   for reducing sedimentation in a beverage.

In one aspect there is provided use of a composition comprising
(i) a food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 800 kDa, or at least 700 kDa, such as at least about 710, 720, 730, 740, 750, 760, 770, 780, or 790 kDa; and
(ii) (1) a protein or (2) a polysaccharide selected from galactomannans, glucomannans, and mixtures thereof;
   for reducing sedimentation in a beverage.

### BRIEF DESCRIPTION OF THE DRAWINGS

The present invention will now be described in further detail by way of example only with reference to the accompanying figure in which:

FIG. 1 shows LUMiSizer plots of chocolate milk, LUMiSizer measurements were done in triplicate shown from top to bottom for each formulation. From left to right: without stabilizer, seaweed flour (sample 1.5) at 800 ppm, control (200 ppm GRINDSTED® Carrageenan CL 220), control (200 ppm refined kappa carrageenan).

FIG. 2 shows pictures of chocolate milk. From left to right: without stabilizer, seaweed flour (sample 1.5) at 800 ppm, control (200 ppm GRINDSTED® Carrageenan CL 220), control (200 ppm refined kappa carrageenan).

FIG. 3 shows seaweed flour (sample 25.5a) dosed between 500 and 3000 ppm, compared to GRINDSTED® Gellan VEG 200 at 330 ppm in an almond chocolate plant-based beverage model system.

FIG. 4 shows seaweed flour (sample 25.5a) dosed between 500 and 2000 ppm, compared to GRINDSTED® Gellan VEG 200 at 330 ppm in a soy protein isolate (SUPRO® XT 55 IP) based model system.

FIG. 5 shows seaweed flour (sample 25.5a) dosed between 500 and 2000 ppm, compared GRINDSTED® Gellan VEG 200 at 330 ppm in a pea protein (TRUPRO™ 2000) based model system.

FIG. 6 shows cooling curves (depicted on the left) and flow curves (depicted on the right) of cocoa model system. Seaweed flour (sample 25.5a) is dosed at 1000 ppm, without protein present (top graph), in combination with 3.4 wt. % Soy protein isolate (SUPRO® XT 55 IP) (middle two graphs) and in combination with 3.4 wt. % Pea protein (TRUPRO™ 2000) (bottom two graphs).

FIG. 7 shows cooling curves (depicted on the left) and flow curves (depicted on the right) of cocoa model system. Seaweed flour (sample 25.5a) is dosed at 1000 ppm with three different Soy protein isolates at 3.4 wt. %, from top to bottom SUPRO® 760 IP, SUPRO® XT 55 IP, SUPRO® XT 221D IP.

FIG. 8 shows cooling curves (depicted on the left) and flow curves (depicted on the right) of cocoa model system. From top to bottom: seaweed flour (sample 25.5a) is dosed at 1000 ppm, without galacto-/gluco-mannans (top graph), in combination with 1000 ppm GRINDSTED® LBG 246, in combination with 1000 ppm GRINDSTED® Guar 250, in combination with 1000 ppm konjac flour (Konjac Gum YZ-J-11 available from Hubei Yizhi Konjac Biotechnology Co., Ltd.).

FIG. 9 shows pictures of soy protein isolate (SUPRO® 760 IP) based soy drinks after 3 months of storage at 4° C. From left to right: control (300 ppm refined iota carrageenan), blank, 150 ppm seaweed flour (sample 1.5), 300 ppm seaweed flour (sample 1.5), 450 ppm seaweed flour (sample 1.5), 600 ppm seaweed flour (sample 1.5), 750 ppm seaweed flour (sample 1.5) and 900 ppm seaweed flour (sample 1.5). Signs of instability/phase separation are indicated by the orange arrows.

FIG. 10 shows LUMiSizer plots of soy protein isolate (SUPRO® 760 IP) based soy drinks, LUMiSizer measurements were done in triplicate shown from top to bottom for each formulation. From left to right: control (300 ppm refined iota carrageenan), blank, 150 ppm seaweed flour (sample 1.5), 300 ppm seaweed flour (sample 1.5), 450 ppm seaweed flour (sample 1.5), 600 ppm seaweed flour (sample 1.5), 750 ppm seaweed flour (sample 1.5) and 900 ppm seaweed flour (sample 1.5). As shown in FIG. 9, stability is achieved at seaweed flour (sample 1.5) levels of 600 ppm and higher and in the 300 ppm refined iota carrageenan control.

FIG. 11 shows cooling curves and flow curves of both seaweed flour (sample 25.5a) with whey protein concentrate in cocoa model system.

FIG. 12 shows cooling curves and flow curves of 1000 ppm seaweed flour (sample 25.5a) with 1000 ppm carob flour (Danisco® Pet Tex 2210), 1000 ppm seaweed flour (sample 25.5a) with 1000 ppm Cassia gum Powder E427 available from Indian Hydrocolloids and 1000 ppm seaweed flour (sample 25.5a) with 200 ppm GRINDSTEDO Gellan VEG 200.

FIG. 13 shows pictures of dairy based desserts according to the invention.

FIG. 14 shows pictures of pea based desserts according to the invention.

FIG. 15 shows pictures of soy based desserts according to the invention.

FIG. 16 shows pictures of soy-based cold-cut slices (1 mm and 2 mm) containing refined kappa carrageenan or seaweed flour at different concentrations according to the invention.

FIG. 17 shows pictures of pea-based cold-cut slices (1 mm and 2 mm) containing refined kappa carrageenan or seaweed flour at different concentrations according to the invention.

FIG. 18 shows pictures of soy-based sausages containing refined kappa carrageenan or seaweed flour at different concentrations according to the invention.

FIG. 19 shows the shear stress (Pa, y-axis) of a dairy-based frozen dessert, containing either refined kappa carrageenan, seaweed flour or no texturant, plotted versus the shear rate (1/s, x-axis).

FIG. 20 shows the shear stress (Pa, y-axis) of a soy-based frozen dessert, containing either refined kappa carrageenan, seaweed flour or no texturant, plotted versus the shear rate (1/s, x-axis).

### DETAILED DESCRIPTION OF THE INVENTION

As discussed herein, in one aspect there is provided a food ingredient obtained from seaweed of the class of Rhodophyta wherein (a) the food ingredient contains mu carrageenan in an amount of at least 4 wt. % based on the total weight of the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa.

The majority of foodstuffs are normally comprised of several ingredients. For example, plant-based beverages are normally comprised of a base (e.g. (whole) soy, almond (butter), (fermented) oat,), vegetable oil (e.g. sunflower oil, canola oil,), a main flavour (e.g. cocoa, coffee, hazelnut,), protein fortification from concentrates and/or isolates (e.g. soy, pea, faba,) and calcium fortification (e.g. calcium carbonate (CaCO₃), tricalcium phosphate (Ca₃(PO₄)₂)). The beverage is then optimized to taste with sugar and salt. Normally, a mixture of these ingredients will result in precipitation and/or creaming effects in the final beverage. The extent of precipitation depends on the ingredient mix. For example, the base, the protein fortification, the calcium fortification and particulate flavours such as cocoa powder are often found to readily precipitate. The extent of creaming depends on the amount of oil/fat (for example coming from vegetable oil or cocoa powder) and on the amount of naturally present emulsifying ingredients in the final beverage. It is also found in plant-based beverages that the low solid content in the final beverage may often result in a lack of mouthfeel, especially when compared to the sensory experience of their non-plant counterparts (e.g. yoghurt drinks, milk drinks).

Since consumer experience is key, it is important for food and beverage manufacturers to be able to provide products such as plant-based beverages that look appealing during their shelf-life and fulfil consumer expectation from a sensory perspective. The requirement to remain appealing applies both at refrigerated and ambient temperatures but ambient temperature can put additional stresses on the beverage system and exaggerate instabilities that are less pronounced in refrigerated conditions.

We have found that an advantageous food ingredient may be provided which provides effective stabilisation of food products such as providing effective reduction in sedimentation in beverages, in particular in plant-based beverages. This food ingredient is obtained from seaweed of the class of Rhodophyta and therefore in view of its natural source it will be seen positively by consumers. In particular, the food ingredient requires no chemical modification and therefore will not be seen as a “chemical additive” by consumers who have a desire for natural products. Furthermore, natural products are regarded positively by regulators. We have identified that a particular component from seaweed of the class of Rhodophyta exhibits these stabilization properties. More specifically, the component surprisingly found to exhibit these properties contains mu (p) carrageenan in an amount of at least 4 wt. % based on the total weight of the food ingredient, and the weight average molecular weight of carrageenan present in the food ingredient is at least 800 kDa, or at least 700 kDa, such as at least about 710, 720, 730, 740, 750, 760, 770, 780, or 790 kDa. This particular component has been shown to be at least as effective in stabilization of food systems, such as plant-based beverages, as prior chemically modified stabilizers. Therefore, by the provision of the present component, effective stabilization may be achieved while reducing the reliance on the food and beverage industry for chemically modified food ingredients.

The present stabilizer, which in some applications may function as a texturant, avoids the need to use food additives such as are typically used in systems requiring stabilization. For example, plant-based drinks are often stabilized with food additives such as gellan gum, locust bean gum, guar gum, xanthan gum, carboxymethyl cellulose, microcrystalline cellulose, carrageenan, starch, modified starch, maltodextrins, lecithin and mono-/di-glycerides. Since one food additive is often not effective in providing all the desired functionality, a combination of the food additives listed above will be required to provide the desired stabilization, emulsification and improved mouthfeel. In contrast, the present food ingredient may provide multiple functionality itself. Therefore, the present invention may provide not only stable and palatable beverages, but addresses increasing consumer demand for more understandable food labelling. Furthermore, the present food ingredient addresses the concern of consumers with regard to food ingredient compositions. In particular, the present food ingredient is advantageous for consumers that are health and environment conscious and who can be put off by the nature of the chemically derived of food additives that are listed on the final product. The present invention addresses the increased demand for clean label ingredients from ingredient manufacturers stemming from more immediately and obviously natural raw material sources.

We have found that a particular preferred aspect of the present invention relates to a combination of the food ingredient of the present invention and protein. The protein may be, for example, a protein concentrate or protein isolate. When combined with a protein the food ingredient of the present invention provides effects such as improved stability, improved viscosity retention and/or improved mouth feel which exceed the effects which would be expected from an additive combination of the present food ingredient and protein. These effects are particularly pronounced when the protein is pea protein or alternatively soy protein. Thus, in a further aspect the present invention provides a composition comprising (i) a food ingredient as defined herein; and (ii) pea protein. Thus in a further aspect the present invention provides a composition comprising (i) a food ingredient obtained from seaweed of the class of Rhodophyta wherein (a) the food ingredient contains mu carrageenan in an amount of at least 4 wt. % based on the total weight of the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa; and (ii) pea protein.

For ease of reference, these and further aspects of the present invention are now discussed under appropriate section headings. However, the teachings under each section are not necessarily limited to each particular section.

The inventors of the present invention have found that the food products obtained from the combination of ingredients described here have some unique properties in terms of elasticity, syneresis control, moisture control, water binding/holding capacity, starch reduction, starch synergy, texture/structure mouthfeel, reduced dosage with synergy.

### Seaweed—Rhodophyta

As discussed herein, the food ingredient is obtained from seaweed of the class of Rhodophyta. Red seaweeds taxonomically belong to the class of Rhodophyta. The seaweed may be referred to as ‘red seaweed’. Examples of suitable seaweeds belong to the genera consisting of Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus.

The food ingredient is obtained from seaweed of the class of Rhodophyta. By the term “obtained from” it is meant that the ingredient is obtained from seaweed of the class of Rhodophyta without chemically modifying the components obtained from the seaweed. The food ingredient obtained from seaweed of the class of Rhodophyta is however treated to obtain it from the seaweed, for example the treatment may deodorize the food ingredient, may leach out certain components or may modify the sodium, potassium or chloride content of the food ingredient.

In one aspect, the seaweed is at least one seaweed of the genera Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus. In one aspect, the seaweed is at least of the genus Eucheuma or Kappaphycus. As will be understood by one skilled in the art, the genus Eucheuma has recently been renamed as Kappaphycus. Therefore, references to the genus Eucheuma may be equivalent to the genus Kappaphycus. In one aspect, the seaweed is at least of the genus Eucheuma. In one aspect, the seaweed is at least of the genus Kappaphycus.

In one aspect, the seaweed is at least of the species Euchema striatum, Kappaphycus striatus (also known as Kappaphycus striatum), Euchema alvarezii, Kappaphycus alvarezii, or a combination thereof. As discussed above, the genus Eucheuma may be equivalent to the genus Kappaphycus. Therefore, references to Euchema striatum may be equivalent to Kappaphycus striatus, and references to Euchema alvarezii may be equivalent to Kappaphycus alvarezii. In one aspect, the seaweed is at least of the species Euchema striatum/Kappaphycus striatus, Euchema alvarezii/Kappaphycus alvarezii, or a combination thereof. In one aspect, the seaweed is at least of the species Euchema striatus. In one aspect, the seaweed is at least of the species Euchema cottonii (also known as Red guso). In one aspect, the seaweed is at least of the species Kappaphycus striatus. In one aspect, the seaweed is at least of the species Euchema alvarezii. In one aspect, the seaweed is at least of the species Kappaphycus alvarezii. In one aspect, the seaweed is at least of the species Euchema striatum/Kappaphycus striatus. In one aspect, the seaweed is at least of the species Euchema alvarezii/Kappaphycus alvarezii. In one aspect, the seaweed is a combination of the species Kappaphycus striatus and Kappaphycus alvarezii, this combination may be known commercially as cottonii.

The food ingredient of the present invention may be provided in the form of a flour. The food ingredient is obtained from seaweed of the class of Rhodophyta and therefore the flour may be referred to as “red seaweed flour”. The term “red seaweed flour” is to be understood as a description of a flour-like product obtained from seaweed of the Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus genera.

The food ingredient of the present invention may be treated to reduce the number of micro-organisms. In one aspect, the food ingredient of the present invention is heat treated to reduce the number of micro-organisms. In one aspect, the food ingredient of the present invention is pasteurized.

In one aspect, the food ingredient of the present invention is deodorized. In one aspect, the food ingredient of the present invention is bleached i.e. its color is reduced. It is to be understood that when referred to “bleached”, this is in one important embodiment a non-chemical bleaching, such as color reduction by natural sunlight. Accordingly, in some embodiment the food ingredient of the present invention is treated with natural sunlight for reducing the color.

In one aspect, the food ingredient of the present invention is dried. In one aspect, the food ingredient of the present invention is milled. In one aspect, the food ingredient of the present invention is dried and milled.

### Carrageenan

As will be appreciated by one skilled in the art, a food ingredient which is obtained from seaweed of the class of Rhodophyta will contain carrageenan. Carrageenan refers to a family of linear sulfated polysaccharides that are extracted from red edible seaweeds. Carrageenan is a high-molecular-weight polysaccharide made up of repeating galactose units and 3,6 anhydrogalactose (3,6-AG), both sulfated and nonsulfated. The units are joined by alternating α-1,3 and β-1,4 glycosidic linkages. Carrageenan is widely used in the food and other industries as thickening or stabilizing agents. There are three main commercial classes of carrageenan:
These three varieties differ in their degree of sulfation. Kappa carrageenan has one sulfate group per disaccharide, iota carrageenan has two, and lambda carrageenan has three. When used in food products, carrageenan has the EU additive E numbers E407 or E407a when present as “processed eucheuma seaweed”.

Kappa carrageenan forms strong, rigid gels in the presence of potassium ions, and reacts with dairy proteins. It is sourced mainly from Kappaphycus alvarezii. Kappa carrageenan is typically produced by alkaline elimination from mu carrageenan, also isolated from Kappaphycus alvarezii. The structures of these two materials and the alkaline conversion is shown below.

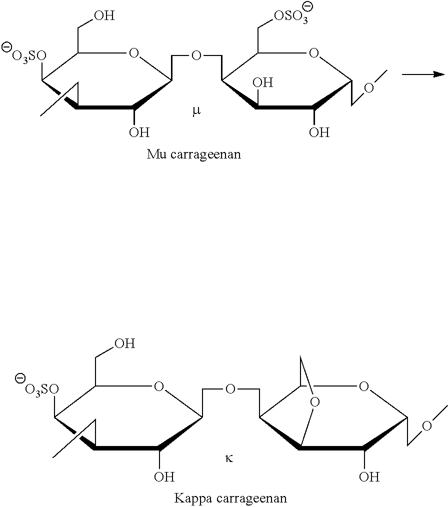

We have identified that by avoiding or reducing this chemical modification i.e. avoiding or reducing alkaline elimination of mu carrageenan (to provide kappa carrageenan) a food ingredient may be provided having the advantageous properties described herein. There is provided a food ingredient having mu carrageenan in an amount of at least 4 wt. % based on the total weight of the food ingredient, and having an weight average molecular weight of carrageenan of at least 800 kDa, or at least 700 kDa, such as at least about 710, 720, 730, 740, 750, 760, 770, 780, or 790 kDa.

The content of the various forms of carrageenan can be readily determined by one skilled in the art. The examples of the present specification provide a detailed method of how this determination may be made. In one aspect, the content of carrageenan type, such a mu carrageenan is determined in accordance with Determination Process 1 (Determination of Carrageenan types by ¹H-NMR).

In one aspect, the food ingredient contains mu carrageenan in an amount of at least 4.5 wt. % based on the total weight of the food ingredient. In one aspect, the food ingredient contains mu carrageenan in an amount of at least 5 wt. % based on the total weight of the food ingredient. In one aspect, the food ingredient contains mu carrageenan in an amount of at least 5.5 wt. % based on the total weight of the food ingredient. In one aspect, the food ingredient contains mu carrageenan in an amount of at least 6 wt. % based on the total weight of the food ingredient. In one aspect, the food ingredient contains mu carrageenan in an amount of at least 6.5 wt. % based on the total weight of the food ingredient. In one aspect, the food ingredient contains mu carrageenan in an amount of at least 7 wt. % based on the total weight of the food ingredient. In one aspect, the food ingredient contains mu carrageenan in an amount of at least 7.5 wt. % based on the total weight of the food ingredient. In one aspect, the food ingredient contains mu carrageenan in an amount of at least 8 wt. % based on the total weight of the food ingredient.

In one aspect, the food ingredient contains mu carrageenan in an amount of no greater than 30 wt. % based on the total weight of the food ingredient. In one aspect, the food ingredient contains mu carrageenan in an amount of no greater than 25 wt. % based on the total weight of the food ingredient. In one aspect, the food ingredient contains mu carrageenan in an amount of no greater than 20 wt. % based on the total weight of the food ingredient. In one aspect, the food ingredient contains mu carrageenan in an amount of no greater than 19 wt. % based on the total weight of the food ingredient. In one aspect, the food ingredient contains mu carrageenan in an amount of no greater than 18 wt. % based on the total weight of the food ingredient. In one aspect, the food ingredient contains mu carrageenan in an amount of no greater than 17 wt. % based on the total weight of the food ingredient. In one aspect, the food ingredient contains mu carrageenan in an amount of no greater than 16 wt. % based on the total weight of the food ingredient. In one aspect, the food ingredient contains mu carrageenan in an amount of no greater than 15 wt. % based on the total weight of the food ingredient. In one aspect, the food ingredient contains mu carrageenan in an amount of no greater than 14 wt. % based on the total weight of the food ingredient. In one aspect, the food ingredient contains mu carrageenan in an amount of no greater than 13 wt. % based on the total weight of the food ingredient.

In one aspect, the food ingredient contains mu carrageenan in an amount of from 4 to 20 wt. % based on the total weight of the food ingredient. In one aspect, the food ingredient contains mu carrageenan in an amount of from 4.5 to 20 wt. % based on the total weight of the food ingredient. In one aspect, the food ingredient contains mu carrageenan in an amount of from 5 to 20 wt. % based on the total weight of the food ingredient. In one aspect, the food ingredient contains mu carrageenan in an amount of from 5.5 to 20 wt. % based on the total weight of the food ingredient. In one aspect, the food ingredient contains mu carrageenan in an amount of from 6 to 20 wt. % based on the total weight of the food ingredient. In one aspect, the food ingredient contains mu carrageenan in an amount of from 6.5 to 20 wt. % based on the total weight of the food ingredient. In one aspect, the food ingredient contains mu carrageenan in an amount of from 7 to 20 wt. % based on the total weight of the food ingredient. In one aspect, the food ingredient contains mu carrageenan in an amount of from 7.5 to 20 wt. % based on the total weight of the food ingredient. In one aspect, the food ingredient contains mu carrageenan in an amount of from 8 to 20 wt. % based on the total weight of the food ingredient.

In one aspect, the food ingredient contains mu carrageenan in an amount of from 4 to 30 wt. % based on the total weight of the food ingredient. In one aspect, the food ingredient contains mu carrageenan in an amount of from 4 to 25 wt. % based on the total weight of the food ingredient. In one aspect, the food ingredient contains mu carrageenan in an amount of from 4 to 20 wt. % based on the total weight of the food ingredient. In one aspect, the food ingredient contains mu carrageenan in an amount of from 4 to 19 wt. % based on the total weight of the food ingredient. In one aspect, the food ingredient contains mu carrageenan in an amount of from 4 to 18 wt. % based on the total weight of the food ingredient. In one aspect, the food ingredient contains mu carrageenan in an amount of from 4 to 17 wt. % based on the total weight of the food ingredient. In one aspect, the food ingredient contains mu carrageenan in an amount of from 4 to 16 wt. % based on the total weight of the food ingredient. In one aspect, the food ingredient contains mu carrageenan in an amount of from 4 to 15 wt. % based on the total weight of the food ingredient. In one aspect, the food ingredient contains mu carrageenan in an amount of from 4 to 14 wt. % based on the total weight of the food ingredient. In one aspect, the food ingredient contains mu carrageenan in an amount of from 4 to 13 wt. % based on the total weight of the food ingredient.

In one aspect, the food ingredient contains mu carrageenan in an amount of at least 6 wt.c/o based on the total weight of carrageenan present in the food ingredient. In one aspect, the food ingredient contains mu carrageenan in an amount of at least 8 wt. % based on the total weight of carrageenan present in the food ingredient. In one aspect, the food ingredient contains mu carrageenan in an amount of at least 10 wt. % based on the total weight of carrageenan present in the food ingredient. In one aspect, the food ingredient contains mu carrageenan in an amount of at least 12 wt. % based on the total weight of carrageenan present in the food ingredient. In one aspect, the food ingredient contains mu carrageenan in an amount of at least 13 wt. % based on the total weight of carrageenan present in the food ingredient.

In one aspect, the food ingredient contains mu carrageenan in an amount of no greater than 30 wt. % based on the total weight of carrageenan present in the food ingredient. In one aspect, the food ingredient contains mu carrageenan in an amount of no greater than 25 wt. % based on the total weight of carrageenan present in the food ingredient. In one aspect, the food ingredient contains mu carrageenan in an amount of no greater than 24 wt. % based on the total weight of carrageenan present in the food ingredient. In one aspect, the food ingredient contains mu carrageenan in an amount of no greater than 22 wt. % based on the total weight of carrageenan present in the food ingredient. In one aspect, the food ingredient contains mu carrageenan in an amount of no greater than 20 wt. % based on the total weight of carrageenan present in the food ingredient.

In one aspect, the present invention provides a food ingredient obtained from seaweed of the class of Rhodophyta wherein
(a) the food ingredient contains mu carrageenan in an amount of at least 6 wt. % based on the total weight of carrageenan present in the food ingredient; and
(b) the weight average molecular weight of carrageenan present in the food ingredient is at least 800 kDa, or at least 700 kDa, such as at least about 710, 720, 730, 740, 750, 760, 770, 780, or 790 kDa. Each of the aspects discussed herein applies to this aspect of the invention.

As discussed herein, the weight average molecular weight of carrageenan present in the food ingredient is at least 800 kDa, or at least 700 kDa, such as at least about 710, 720, 730, 740, 750, 760, 770, 780, or 790 kDa. We have identified that it is important to maintain carrageenan in its native form or close to its native form, as part of the seaweed structure. In addition to avoiding or reducing chemical modification of mu carrageenan to provide kappa carrageenan, it is also important to maintain a relatively high weight average molecular weight. By provision of both the relatively high mu carrageenan content and the weight average molecular weight of at least 700 KDa or at least 800 kDa a food ingredient is provided having the advantageous properties described herein.

The weight average molecular weight is determined based on the number of molecules present and the weight of each molecule. The weight average molecular weight is given by:

$$weight average molecular weight = \frac{{\sum }_{i = 1}^{∞} NiMiMi}{{\sum }_{i = 1}^{∞} NiMi}$$

Weight average molecular weight can also be expressed as:
weight average molecular weight=Σᵢ₌₁^{∞}wiMi
where wi is the weight fraction of polymer with molecular weight Mi.

The weight average molecular weight may be determined by standard Multi Angle Laser Light Scattering (MALLS) techniques. In one aspect the weight average molecular weight is determined in accordance with Determination Process 2.

The weight average molecular weight of carrageenan can be readily determined by one skilled in the art. The examples of the present specification provide a detailed method of how this determination may be made. In one aspect, the weight average molecular weight of carrageenan is determined in accordance with Determination Process 2 (Determination of Weight average molecular weight).

In one aspect the weight average molecular weight of carrageenan present in the food ingredient is at least 700 kDa. In one aspect the weight average molecular weight of carrageenan present in the food ingredient is at least 710 kDa. In one aspect the weight average molecular weight of carrageenan present in the food ingredient is at least 720 kDa. In one aspect the weight average molecular weight of carrageenan present in the food ingredient is at least 730 kDa. In one aspect the weight average molecular weight of carrageenan present in the food ingredient is at least 740 kDa. In one aspect the weight average molecular weight of carrageenan present in the food ingredient is at least 750 kDa. In one aspect the weight average molecular weight of carrageenan present in the food ingredient is at least 760 kDa. In one aspect the weight average molecular weight of carrageenan present in the food ingredient is at least 770 kDa. In one aspect the weight average molecular weight of carrageenan present in the food ingredient is at least 780 kDa. In one aspect the weight average molecular weight of carrageenan present in the food ingredient is at least 790 kDa. In one aspect the weight average molecular weight of carrageenan present in the food ingredient is at least 800 kDa, 810, 820, 830, or 840 kDa. In one aspect the weight average molecular weight of carrageenan present in the food ingredient is at least 850 kDa. In one aspect the weight average molecular weight of carrageenan present in the food ingredient is at least 900 kDa. In one aspect the weight average molecular weight of carrageenan present in the food ingredient is at least 950 kDa. In one aspect the weight average molecular weight of carrageenan present in the food ingredient is at least 1000 kDa. In one aspect the weight average molecular weight of carrageenan present in the food ingredient is at least 1050 kDa. In one aspect the weight average molecular weight of carrageenan present in the food ingredient is at least 1100 kDa. In one aspect the weight average molecular weight of carrageenan present in the food ingredient is at least 1150 kDa. In one aspect the weight average molecular weight of carrageenan present in the food ingredient is at least 1200 kDa.

In one aspect the weight average molecular weight of carrageenan present in the food ingredient is no greater than 3000 kDa. In one aspect the weight average molecular weight of carrageenan present in the food ingredient is no greater than 2800 kDa. In one aspect the weight average molecular weight of carrageenan present in the food ingredient is no greater than 2600 kDa. In one aspect the weight average molecular weight of carrageenan present in the food ingredient is no greater than 2400 kDa. In one aspect the weight average molecular weight of carrageenan present in the food ingredient is no greater than 2200 kDa. In one aspect the weight average molecular weight of carrageenan present in the food ingredient is no greater than 2000 kDa. In one aspect the weight average molecular weight of carrageenan present in the food ingredient is no greater than 1800 kDa. In one aspect the weight average molecular weight of carrageenan present in the food ingredient is no greater than 1700 kDa. In one aspect the weight average molecular weight of carrageenan present in the food ingredient is no greater than 1600 kDa. In one aspect the weight average molecular weight of carrageenan present in the food ingredient is no greater than 1500 kDa.

In one aspect the weight average molecular weight of carrageenan present in the food ingredient is from 700 to 3000 (such as 750 to 2000) kDa. In one aspect the weight average molecular weight of carrageenan present in the food ingredient is from 850 to 3000 (such as 850 to 2000) kDa. In one aspect the weight average molecular weight of carrageenan present in the food ingredient is from 900 to 3000 (such as 900 to 2000) kDa. In one aspect the weight average molecular weight of carrageenan present in the food ingredient is from 950 to 3000 (such as 950 to 2000) kDa. In one aspect the weight average molecular weight of carrageenan present in the food ingredient is from 1000 to 3000 (such as 1000 to 2000) kDa. In one aspect the weight average molecular weight of carrageenan present in the food ingredient is from 1050 to 3000 (such as 1050 to 2000) kDa. In one aspect the weight average molecular weight of carrageenan present in the food ingredient is from 1100 to 3000 (such as 1100 to 2000) kDa. In one aspect the weight average molecular weight of carrageenan present in the food ingredient is from 1150 to 3000 (such as 1150 to 2000) kDa. In one aspect the weight average molecular weight of carrageenan present in the food ingredient is from 1200 to 3000 (such as 1200 to 2000) kDa.

In one aspect the weight average molecular weight of carrageenan present in the food ingredient is from 700 to 3000 kDa. In one aspect the weight average molecular weight of carrageenan present in the food ingredient is from 800 to 3000 (such as from 1000 to 3000) kDa. In one aspect the weight average molecular weight of carrageenan present in the food ingredient is from 800 to 2800 (such as from 1000 to 2800) kDa. In one aspect the weight average molecular weight of carrageenan present in the food ingredient is from 800 to 2600 (such as from 1000 to 2600) kDa. In one aspect the weight average molecular weight of carrageenan present in the food ingredient is from 800 to 2400 (such as from 1000 to 2400) kDa. In one aspect the weight average molecular weight of carrageenan present in the food ingredient is from 800 to 2200 (such as from 1000 to 2200) kDa. In one aspect the weight average molecular weight of carrageenan present in the food ingredient is from 800 to 2000 (such as from 1000 to 2000) kDa. In one aspect the weight average molecular weight of carrageenan present in the food ingredient is from 800 to 1800 (such as from 1000 to 1800) kDa. In one aspect the weight average molecular weight of carrageenan present in the food ingredient is from 800 to 1700 (such as from 1000 to 1700) kDa. In one aspect the weight average molecular weight of carrageenan present in the food ingredient is from 800 to 1600 (such as from 1000 to 1600) kDa. In one aspect the weight average molecular weight of carrageenan present in the food ingredient is from 800 to 1500 (such as from 1000 to 1500) kDa.

It is desirable to provide the advantageous product of the present invention to limit the total amount of kappa carrageenan. In particular, since kappa carrageenan forms particularly strong gels it may “over stabilize” food and beverage products. For example, for a beverage it is desirable to stabilize the product to the extent that particulate material does not form a sediment during storage. However, if the product is over stabilized then achieving an even dispersion of the particulate product during production is difficult. In particular, a gel can be formed to quickly before the particulate material has been dispersed evenly within the product. In one aspect, the food ingredient contains kappa carrageenan in an amount of no greater than 60 wt. % based on the total weight of the food ingredient. In one aspect, the food ingredient contains kappa carrageenan in an amount of no greater than 55 wt. % based on the total weight of the food ingredient.

As discussed herein, the content of the various forms of carrageenan can be readily determined by one skilled in the art. The examples of the present specification provide a detailed method of how this determination may be made. In one aspect, the content of carrageenan type, such a kappa carrageenan, is in accordance with Determination Process 1 (Determination of Carrageenan types by 1H-NMR).

In one aspect, the food ingredient contains kappa carrageenan in an amount of from 20 to 70 wt. %, such as from 20 to 65 wt. %, such as from 40 to 60 wt. %, such as from 50 to 60 wt. %, or from 20 to 60 wt. % based on the total weight of the food ingredient. In one aspect, the food ingredient contains kappa carrageenan in an amount of from 25 to 60 wt. % based on the total weight of the food ingredient. In one aspect, the food ingredient contains kappa carrageenan in an amount of from 30 to 60 wt. % based on the total weight of the food ingredient. In one aspect, the food ingredient contains kappa carrageenan in an amount of from 35 to 60 wt. % based on the total weight of the food ingredient. In one aspect, the food ingredient contains kappa carrageenan in an amount of from 20 to 55 wt. % based on the total weight of the food ingredient. In one aspect, the food ingredient contains kappa carrageenan in an amount of from 25 to 55 wt. % based on the total weight of the food ingredient. In one aspect, the food ingredient contains kappa carrageenan in an amount of from 30 to 55 wt. % based on the total weight of the food ingredient. In one aspect, the food ingredient contains kappa carrageenan in an amount of from to 55 wt. % based on the total weight of the food ingredient.

In one aspect, the food ingredient contains kappa carrageenan in an amount of from 20 to 70 wt. %, such as from 20 to 65 wt. %, such as from 40 to 60 wt. %, such as from 50 to 60 wt. %, or from 20 to 60 wt. % based on the total weight of carrageenan present in the food ingredient. In one aspect, the food ingredient contains kappa carrageenan in an amount of from 25 to 60 wt. % based on the total weight of carrageenan present in the food ingredient. In one aspect, the food ingredient contains kappa carrageenan in an amount of from 30 to 60 wt. % based on the total weight of carrageenan present in the food ingredient. In one aspect, the food ingredient contains kappa carrageenan in an amount of from 35 to 60 wt. % based on the total weight of carrageenan present in the food ingredient. In one aspect, the food ingredient contains kappa carrageenan in an amount of from 20 to 55 wt. % based on the total weight of carrageenan present in the food ingredient. In one aspect, the food ingredient contains kappa carrageenan in an amount of from 25 to 55 wt. % based on the total weight of carrageenan present in the food ingredient. In one aspect, the food ingredient contains kappa carrageenan in an amount of from 30 to 55 wt. % based on the total weight of carrageenan present in the food ingredient. In one aspect, the food ingredient contains kappa carrageenan in an amount of from 35 to 55 wt. % based on the total weight of carrageenan present in the food ingredient.

### Acid Insoluble Material

We have also identified that the removal of acid insoluble material to provide a chemically modified product is undesirable for the reasons set out herein. Typical chemically modified products obtained from seaweed may have an acid insoluble material content of less than 2%.

We have found that higher contents of acid insoluble material are advantageous in achieving the objects of the present invention.

The term “acid insoluble matter” is well understood by one skilled in the art, at least because it is a regulated parameter. By the term “acid insoluble matter” it is meant the percentage of material left after heating in a steam bath or boiling water bath where the material in question is treated with sulfuric acid. The amount of acid insoluble material is determined by weight as regards the amount of insoluble material retained on a filter from a pre-weighed sample and expressed as a percentage.

The acid insoluble matter can be readily determined by one skilled in the art. The examples of the present specification provide a detailed method of how this determination may be made. In one aspect, the acid insoluble matter is determined in accordance with Determination Process 3 (Determination of Acid Insoluble Matter).

In one aspect, the food ingredient contains acid insoluble matter in an amount of at least 3 wt. % based on the total weight of the food ingredient. In one aspect, the food ingredient contains acid insoluble matter in an amount of at least 4 wt. % based on the total weight of the food ingredient. In one aspect, the food ingredient contains acid insoluble matter in an amount of at least 5 wt. % based on the total weight of the food ingredient. In one aspect, the food ingredient contains acid insoluble matter in an amount of at least 6 wt. % based on the total weight of the food ingredient. In one aspect, the food ingredient contains acid insoluble matter in an amount of at least 7 wt. % based on the total weight of the food ingredient. In one aspect, the food ingredient contains acid insoluble matter in an amount of at least 8 wt. % based on the total weight of the food ingredient. In one aspect, the food ingredient contains acid insoluble matter in an amount of at least 8.5 wt. % based on the total weight of the food ingredient.

In one aspect, the food ingredient contains acid insoluble matter in an amount of no greater than 20 wt. % based on the total weight of the food ingredient. In one aspect, the food ingredient contains acid insoluble matter in an amount of no greater than 18 wt. % based on the total weight of the food ingredient. In one aspect, the food ingredient contains acid insoluble matter in an amount of no greater than 16 wt. % based on the total weight of the food ingredient. In one aspect, the food ingredient contains acid insoluble matter in an amount of no greater than 15 wt. % based on the total weight of the food ingredient. In one aspect, the food ingredient contains acid insoluble matter in an amount of no greater than 14 wt. % based on the total weight of the food ingredient. In one aspect, the food ingredient contains acid insoluble matter in an amount of no greater than 13 wt. % based on the total weight of the food ingredient. In one aspect, the food ingredient contains acid insoluble matter in an amount of no greater than 12 wt. % based on the total weight of the food ingredient. In one aspect, the food ingredient contains acid insoluble matter in an amount of no greater than 11 wt. % based on the total weight of the food ingredient. In one aspect, the food ingredient contains acid insoluble matter in an amount of no greater than 10.5 wt. % based on the total weight of the food ingredient.

In one aspect, the food ingredient contains acid insoluble matter in an amount of from 3 to 20 wt. % based on the total weight of the food ingredient. In one aspect, the food ingredient contains acid insoluble matter in an amount of from 4 to 20 wt. % based on the total weight of the food ingredient. In one aspect, the food ingredient contains acid insoluble matter in an amount of from 5 to 20 wt. % based on the total weight of the food ingredient. In one aspect, the food ingredient contains acid insoluble matter in an amount of from 6 to 20 wt. % based on the total weight of the food ingredient. In one aspect, the food ingredient contains acid insoluble matter in an amount of from 7 to 20 wt. % based on the total weight of the food ingredient. In one aspect, the food ingredient contains acid insoluble matter in an amount of from 8 to 20 wt. % based on the total weight of the food ingredient. In one aspect, the food ingredient contains acid insoluble matter in an amount of from 8.5 to 20 wt. % based on the total weight of the food ingredient.

In one aspect, the food ingredient contains acid insoluble matter in an amount of from 3 to 18 wt. % based on the total weight of the food ingredient. In one aspect, the food ingredient contains acid insoluble matter in an amount of from 3 to 16 wt. % based on the total weight of the food ingredient. In one aspect, the food ingredient contains acid insoluble matter in an amount of from 3 to 15 wt. % based on the total weight of the food ingredient. In one aspect, the food ingredient contains acid insoluble matter in an amount of from 3 to 14 wt. % based on the total weight of the food ingredient. In one aspect, the food ingredient contains acid insoluble matter in an amount of from 3 to 13 wt. % based on the total weight of the food ingredient. In one aspect, the food ingredient contains acid insoluble matter in an amount of from 3 to 12 wt. % based on the total weight of the food ingredient. In one aspect, the food ingredient contains acid insoluble matter in an amount of from 3 to 11 wt. % based on the total weight of the food ingredient. In one aspect, the food ingredient contains acid insoluble matter in an amount of from 3 to 10.5 wt. % based on the total weight of the food ingredient.

In one aspect, the food ingredient contains acid insoluble matter in an amount of from 5 to 15 wt. % based on the total weight of the food ingredient. In one aspect, the food ingredient contains acid insoluble matter in an amount of from 8 to 15 wt. % based on the total weight of the food ingredient. In one aspect, the food ingredient contains acid insoluble matter in an amount of from 8.5 to 10.5 wt. % based on the total weight of the food ingredient.

### Compositions

The present invention further provides compositions containing the food ingredient as described herein. The compositions may contain one or more components in addition to the food ingredient as described herein.

In one aspect the present invention provides a composition comprising (i) a food ingredient as defined herein; and (ii) a protein. We have found that the present food ingredient is particularly advantageous in stabilizing protein containing food and beverage products. In particular, the present food ingredient interacts with protein in products such as beverage products to provide products such as a beverage having improved viscosity and/or improved mouth feel when compared the expected viscosity and/or mouth feel which would be expected from the food ingredient and protein having an additive effect.

In one aspect, the protein is a protein concentrate, a protein isolate or a mixture thereof. In one aspect, the protein is a protein concentrate. In one aspect, the protein is a protein isolate. In one aspect, the protein is a mixture of protein concentrate and protein isolate.

The term “protein concentrate” is well understood by one skilled in the art. By the term “protein concentrate” it is meant a composition comprising a protein wherein the protein is present at a concentration greater than found in nature or when synthesized. Typically, a protein concentrate contains fat and carbohydrate fractions.

The term “protein isolate” is well understood by one skilled in the art, By the term “protein isolate” it is meant a composition comprising a purified protein wherein the composition does not contain all of the components found in the protein containing composition in nature or after synthesis.

The protein may be selected from any suitable protein source. In one aspect, the protein is selected from legumenous proteins, such as soy protein, pea protein, faba protein, carob protein, cereal proteins, such as oat protein; grass protein, cotton protein, dairy proteins, such as whey protein, casein protein, and mixtures thereof. In one aspect, the protein is selected from soy protein, pea protein, and mixtures thereof. In one aspect, the protein is at least soy protein. In one aspect, the protein is at least pea protein. In one aspect, the protein is a mixture of soy protein and pea protein.

In one aspect the present invention provides a composition comprising (i) a food ingredient as defined herein; and (ii) pea protein. In one aspect the present invention provides a composition comprising (i) a food ingredient obtained from seaweed of the class of Rhodophyta wherein (a) the food ingredient contains mu carrageenan in an amount of at least 4 wt. % based on the total weight of the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa; and (ii) pea protein.

In one aspect the present invention provides a composition comprising (i) a food ingredient obtained from seaweed of the class of Rhodophyta wherein (a) the food ingredient contains mu carrageenan in an amount of at least 6 wt. % based on the total weight of carrageenan present in the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa; and (ii) pea protein. Each of the aspects discussed herein applies to this aspect of the invention.

Whey protein, a mixture of proteins containing a-lactalbumun, f3-lactoglobulin, serum albumin and immunoglobulins, is commonly used as thickener to improve the texture and exert syneresis control on food systems. Whey Protein Concentrates possess low but still significant quantities of fat and cholesterol, and generally contain higher levels of bioactive components such as carbohydrates (lactose) than the Isolated versions, and they vary in protein content from around to 90%.

Soy protein stems from dehulled and defatted soy bean meal, and the isolated format is highly refined and purified such that the protein concentration is at a minimum moisture free level of 90%. Most of the non-protein components have been removed and this results in an overall neutral flavour, and a tendency to decrease subsequent flatulence on ingesting compared to the less protein pure versions. The primary use of soy protein isolates is as texture improvers of meat alternative products, enhancers in moisture retention and for their emulsification functionality.

Pea protein extracted from green, yellow or split peas contains significant protein content, but the content varies based on the individual plant's genetics and the climatic conditions under which it grew. Its use within foods is predominantly as a nutritional and texture enhancer, and can also be used to optimise viscosity, gelation, stability and emulsifying properties, whilst demonstrating fat binding qualities. Typically, it can be found in isolated or concentrate forms through processing via a wet or dry fractionation method respectively. Containing 8 of the 9 essential amino acids, lacking only methionine, pea protein is considered an incomplete protein.

In one aspect the present invention provides a composition comprising (i) a food ingredient as defined herein; and (ii) a polysaccharide selected from galactomannans, glucomannans, and mixtures thereof. We have found that the present food ingredient is particularly advantageous in stabilizing food and beverage products galactomannans and/or glucomannans. In particular, the present food ingredient interacts with galactomannans and/or glucomannans in products such as beverage products to provide products such as a beverage having improved viscosity and/or improved mouth feel when compared the expected viscosity and/or mouth feel which would be expected from the food ingredient and galactomannans and/or glucomannans having an additive effect.

Galactomannans are examples of polysaccharides, specifically consisting of a mannose backbone along which galactose side chains extend outwards. More specifically the mannose backbone is a 1-4 linked beta-D-mannopyranose chain from which 1-6 linked alpha-D-galactopyranose side units emanate. The frequency of these side units decide which polysaccharide is provided. For the following Galactose:Mannose ratios the gums listed are provided. 1:1=Fenugreek gum, 1:2=Guar gum, 1:3=Tara gum, 1:4=Locust bean gum (LBG), 1:5=Cassia gum. Guar and LBG are typically used to increase the viscosity of the water phase of food products.

The galactomannan may be any suitable galactomannan. In one aspect, the galactomannan is selected from guar gum, tara gum, locust bean gum, carob flour, and mixtures thereof. In one aspect, the galactomannan is selected from guar gum, tara gum, locust bean gum, and mixtures thereof.

Glucomannans are water soluble largely linear polysaccharides with only limited degrees of branching side chains, where the main sugar components are beta-(1-4)-linked D-Mannose and D-glucose side units occurring with an 8% repeating frequency. The typical example of this hemicellulose polysaccharide is Konjac, and its main function within food systems is as an emulsifier and thickener.

The glucomannan may be any suitable glucomannan. In one aspect, the glucomannan is derived from the konjac plant. In one aspect, the glucomannan is obtained from the konjac plant.

### Foodstuff

In a further aspect, the present invention provides a foodstuff containing a food ingredient as defined herein or containing a composition as defined herein. The foodstuff may contain the food ingredient or composition in any suitable amount to achieve the desired properties.

In a further aspect, the present invention provides a foodstuff containing a food ingredient obtained from seaweed of the class of Rhodophyta wherein (a) the food ingredient contains mu carrageenan in an amount of at least 6 wt. % based on the total weight of carrageenan present in the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa. Each of the aspects discussed herein applies to this aspect of the invention.

In a further aspect, the present invention provides a foodstuff containing (i) a food ingredient obtained from seaweed of the class of Rhodophyta wherein (a) the food ingredient contains mu carrageenan in an amount of at least 6 wt. % based on the total weight of carrageenan present in the food ingredient; and (b) the weight average molecular weight of carrageenan present in the food ingredient is at least 800 kDa, or at least 700 kDa, such as at least about 710, 720, 730, 740, 750, 760, 770, 780, or 790 kDa; and (ii) protein, such as pea protein. Each of the aspects discussed herein applies to this aspect of the invention.

In one aspect the food ingredient obtained from seaweed of the class of Rhodophyta is defined as a composition consisting of components selected from the group consisting of NaCl, KCI, protein, fat, carrageenan, dietary fiber, acid insoluble matter, starch, and carbohydrates. In the present specification the amounts of food ingredient present in the foodstuff may refer to the amount of composition obtained from seaweed of the class of Rhodophyta consisting of components selected from the group consisting of NaCl, KCl, protein, fat, carrageenan, dietary fiber, acid insoluble matter, starch, and carbohydrates.

In one aspect, the food ingredient is present in the foodstuff in an amount of no greater than 10 wt. % based on the total weight of the foodstuff. In one aspect, the food ingredient is present in the foodstuff in an amount of no greater than 8 wt. % based on the total weight of the foodstuff. In one aspect, the food ingredient is present in the foodstuff in an amount of no greater than 7 wt. % based on the total weight of the foodstuff. In one aspect, the food ingredient is present in the foodstuff in an amount of no greater than 6 wt. % based on the total weight of the foodstuff. In one aspect, the food ingredient is present in the foodstuff in an amount of no greater than 5 wt. % based on the total weight of the foodstuff. In one aspect, the food ingredient is present in the foodstuff in an amount of no greater than 4 wt. % based on the total weight of the foodstuff. In one aspect, the food ingredient is present in the foodstuff in an amount of no greater than 3 wt. % based on the total weight of the foodstuff.

In one aspect, the food ingredient is present in the foodstuff in an amount of at least 0.01 wt. % based on the total weight of the foodstuff. In one aspect, the food ingredient is present in the foodstuff in an amount of at least 0.02 wt. % based on the total weight of the foodstuff. In one aspect, the food ingredient is present in the foodstuff in an amount of at least 0.05 wt. % based on the total weight of the foodstuff. In one aspect, the food ingredient is present in the foodstuff in an amount of at least 0.1 wt. % based on the total weight of the foodstuff. In one aspect, the food ingredient is present in the foodstuff in an amount of at least 0.2 wt. % based on the total weight of the foodstuff. In one aspect, the food ingredient is present in the foodstuff in an amount of at least 0.5 wt. % based on the total weight of the foodstuff. In one aspect, the food ingredient is present in the foodstuff in an amount of at least 1 wt. % based on the total weight of the foodstuff.

In one aspect, the food ingredient is present in the foodstuff in an amount of from 0.01 to 10 wt. % based on the total weight of the foodstuff. In one aspect, the food ingredient is present in the foodstuff in an amount of from 0.01 to 8 wt. % based on the total weight of the foodstuff. In one aspect, the food ingredient is present in the foodstuff in an amount of from 0.01 to 7 wt. % based on the total weight of the foodstuff. In one aspect, the food ingredient is present in the foodstuff in an amount of from 0.01 to 6 wt. % based on the total weight of the foodstuff. In one aspect, the food ingredient is present in the foodstuff in an amount of from 0.01 to 5 wt. % based on the total weight of the foodstuff. In one aspect, the food ingredient is present in the foodstuff in an amount of from 0.01 to 4 wt. % based on the total weight of the foodstuff. In one aspect, the food ingredient is present in the foodstuff in an amount of from 0.01 to 3 wt. % based on the total weight of the foodstuff.

In one aspect, the food ingredient is present in the foodstuff in an amount of from 0.01 to 5 wt. % (such as from 0.01 to 3 wt. %) based on the total weight of the foodstuff. In one aspect, the food ingredient is present in the foodstuff in an amount of from 0.02 to 5 wt. % (such as from 0.02 to 3 wt. %) based on the total weight of the foodstuff. In one aspect, the food ingredient is present in the foodstuff in an amount of from 0.05 to 5 wt. % (such as from 0.05 to 3 wt. %) based on the total weight of the foodstuff. In one aspect, the food ingredient is present in the foodstuff in an amount of from 0.1 to 5 wt. % (such as from 0.1 to 3 wt. %) based on the total weight of the foodstuff. In one aspect, the food ingredient is present in the foodstuff in an amount of from 0.2 to 5 wt. % (such as from 0.2 to 3 wt. %) based on the total weight of the foodstuff. In one aspect, the food ingredient is present in the foodstuff in an amount of from 0.5 to 5 wt. % (such as from to 3 wt. %) based on the total weight of the foodstuff. In one aspect, the food ingredient is present in the foodstuff in an amount of from 1 to 5 wt. % (such as from 1 to 3 wt. %) based on the total weight of the foodstuff.

In one aspect, the foodstuff is a plant-based foodstuff. As will be understood by one skilled in the art, a plant-based foodstuff is one that contains no ingredients of animal origin.

In one aspect, the foodstuff is a beverage. In one aspect, the foodstuff is a plant-based beverage.

In one aspect, the foodstuff is a non-diary foodstuff i.e. it is not a dairy foodstuff. In one aspect, the foodstuff contains no milk.

In one aspect, when the foodstuff is a dairy foodstuff, such as a mousse or a creamer, dessert, the food ingredient is present in an amount of less than 8.4 wt. %, such as less than 6 wt. %, such as less than 4 wt. %, or less than 2 wt. %, or less than 1 wt. %, or less than 0.5 wt. %, or in the range of 0.02% wt. to 1 wt. %, such as in the range of 0.1% wt. to 1 wt. %, or is present in an amount of greater than 9.3 wt. %, based on the total weight of the foodstuff. In one aspect, when the foodstuff is a dairy foodstuff, the food ingredient is present in an amount of less than 8.4 wt. %, such as less than 6 wt. %, such as less than 4 wt. %, or less than 2 wt. %, or less than 1 wt. %, or less than 0.5 wt. %, or in the range of 0.02% wt. to 1 wt. %, based on the total weight of the foodstuff.

In one aspect, when the foodstuff is a beverage, the food ingredient is present in an amount of less than 3 wt. %, or less than 2 wt. %, or less than 1 wt. %, or less than 0.5 wt. %, or in the range of 0.02% wt. to 1 wt. %, such as in the range of 0.1% wt. to 1 wt. %, based on the total weight of the foodstuff. In one aspect, when the foodstuff is a dairy foodstuff, the food ingredient is present in an amount of less than 3 wt. %, or less than 2 wt. %, or less than 1 wt. %, or less than wt. %, or in the range of 0.02% wt. to 1 wt. %, based on the total weight of the foodstuff.

In one aspect, when the foodstuff is a meat-alternative, the food ingredient is present in an amount of less than 8.4 wt. %, such as less than 6 wt. %, such as less than 4 wt. %, or 3 wt. %, or less than 2 wt. %, or less than 1 wt. %, or less than 0.5 wt. %, or in the range of 0.02% wt. to 1 wt. %, such as in the range of 0.1% wt. to 1 wt. %, based on the total weight of the foodstuff. In one aspect, when the foodstuff is a dairy foodstuff, the food ingredient is present in an amount of less than 3 wt. %, or less than 2 wt. %, or less than 1 wt. %, or less than 0.5 wt. %, or in the range of 0.02% wt. to 1 wt. %, based on the total weight of the foodstuff.

In one aspect, the food ingredient is present in an amount of less than 8.4 wt. %, such as less than 6 wt. %, such as less than 4 wt. %, or less than 2 wt. %, or less than 1 wt. %, or less than 0.5 wt. %, or in the range of 0.02% wt. to 1 wt. %, or is present in an amount of greater than 9.3 wt. %, based on the total weight of the foodstuff. In one aspect, the food ingredient is present in an amount of less than 8.4 wt. % based on the total weight of the foodstuff.

### Food Ingredient

The food ingredient of the present invention may be prepared by any suitable process. In one aspect, the food ingredient is obtained by a process comprising the steps of:
(a) providing seaweed of the class of Rhodophyta,
(b) drying the seaweed of step (a), and optionally reducing color by natural sunlight;
(c) rehydrating the dried seaweed at a temperature of from 20° C. to 85° C., such as from 50° C. to 75° C., or from 60° C. to 70° C. normally in the presence of a salt solution (such as NaCl and/or KCl and/or CaCl 2) at a pH that is lower than 9.5, such as lower than 8.5, such as in the range of 6.5 to 8.5;
(d) separating the rehydrated seaweed of step (c) from the solution;
(e) drying the product of step (d), and
(f) optionally milling the dried product of step (e) to form the food ingredient.

In one aspect the food ingredient of the present invention is prepared by a process as described in International Patent Application No. PCT/US2020/033805.

### Other Aspects

In a further aspect, the present invention provides a method for the preparation of a foodstuff comprising the step of combining with a food material, a food ingredient as defined herein or a composition as defined herein, to provide the foodstuff.

In a further aspect, the present invention provides a method for improving the stability of a foodstuff (such as a beverage), the method comprising the step of combining with the foodstuff a food ingredient as defined herein or a composition as defined herein.

In a further aspect, the present invention provides use of a food ingredient as defined herein or a composition herein for improving the stability of a foodstuff (such as a beverage).

In a further aspect, the present invention provides a method for reducing sedimentation in a beverage, the method comprising the step of combining with the beverage a food ingredient as defined herein or a composition as defined herein.

In a further aspect, the present invention provides use of a food ingredient as defined herein or a composition as defined herein for reducing sedimentation in a beverage.

In a further aspect, the present invention provides a method for thickening a beverage, the method comprising the step of combining with the beverage a food ingredient as defined herein or a composition as defined herein.

In a further aspect, the present invention provides use of a food ingredient as defined herein or a composition as defined herein for thickening a beverage.

In a further aspect, the present invention provides a method for improving the mouthfeel of a foodstuff (such as a beverage), the method comprising the step of combining with the beverage a food ingredient as defined herein or a composition as defined herein.

In a further aspect, the present invention provides use of a food ingredient as defined herein or a composition as defined herein for improving the mouthfeel of a foodstuff (such as a beverage).

The invention will now be described with reference to the following non-limiting examples.

### EXAMPLES

### Methods

### Determination of Carrageenan types by 1 H-NMR (Determination Process 1)

Determination of the presence of different carrageenan types and quantification of the relative amount of each carrageenan (Kappa, Mu, Iota, Nu, Lambda, Theta, Beta and contamination of Starch) by ¹H NMR.

A 0.3w/w % carrageenan sample is made by dissolving 3 mg homogenized carrageenan powder in 1 mL pH3 buffer (with 0.1% 3-(Trimethylsilyl) propionic-2,2,3,3-d4-acid sodium salt (TSP) as the internal reference). The sample is left on a heatblock with agitation for 3 h at 70° C. The sample is recorded in a 5 mm NMR probe and the spectrum is analysed by integration of spectra done automatically by custom made program. The sample has to be washed before ¹H-NMR spectrum is recorded in order to obtain a spectrum with correct quantification results.

KHP buffer is prepared by dissolving 1.021 gr of potassium hydrogen phthalate and 0.1 g TSP in 40 mL D 2 0. pH is set to 3 (+/−0.05) using DCI. When pH has reached the desired value dilute up to 100 mL to give the final 50 mM pH 3 buffer.

### Procedure ¹H-NMR spectrum

### Sample Handling

3 mg +/−0.5 mg
Samples are weighed out into Eppendorf tubes
1 mL of KHP buffer is added and samples are placed on heat block with agitation at 70° C., 1400 rpm for 3 hours
550 μL is transferred to NMR tubes for NMR analysis

### Recording of ¹H-NMR Spectrum 5 mm BBO Smartprobe:

Pulseq: zg
Ns=64
D1=20 sec
aq=2.6 sec
sw=20 ppm,
O1p=6.175,
Temp=353K
Run from IconNMR
Solvent: D2O_Hydroc
Parameterset: QUANTHYDROCOLLOID

### Recording of ¹H-NMR Spectrum 5 mm TCI Prodigy Cryo-Probe:

Pulseq: zg
Ns=64
D1=20 sec
aq=2.6 sec
sw=20 ppm,
O1p=6.175,
Temp=333K
Run from IconNMR
Solvent: D20
Parameterset: QUANTHYDROCOLLOID.cryo

### Procedure HSQC-NMR spectrum

### Sample Handling

50 +/−5 mg
Samples are weighed out into Eppendorf tubes
1mL of KHP buffer is added and samples are placed on heat block with agitation at 70° C., 1400 rpm overnight.
550 μL is transferred to NMR tubes for NMR analysis

### Recording of HSQC-NMR Spectrum 5 mm BBO Smartprobe:

Pulseq: hsqcedetgpsisp2.3
Ns=16
D1=1 sec
aq=0.57sec
sw1p=2.99 ppm
O1p=4.7
sw2p=165 ppm
O2p=75 ppm
Temp=353K
Run from IconNMR
Solvent: D2O_Hydroc
Parameterset: HSQC_carrageenan D2O Hydroc

The principle of the analysis is as follows:
1. Each spectrum is integrated in predefined regions

| Peak | Start (ppm) | Stop (ppm) | Mw(g/mol)* |
|---|---|---|---|
| IS | −0.200 | 0.200 | 172.27 |
| beta | 5.0694 | 5.0885 | 305 |
| kappa | 5.0885 | 5.127 | 420 |
| mu | 5.22 | 5.256 | 558 |
| theta | 5.298 | 5.256 | 541 |
| iota | 5.345 | 5.294 | 541 |
| starch | 5.340 | 5.400 | 162 |
| nu | 5.523 | 5.47 | 628 |
| lambda | 5.523 | 5.60 | 580 |

Calculation: The mass of each carrageenan type is calculated based on the integral (A) of the carrageenan and the internal standard, the number of contributing protons(nH), the molecular weight of each carrageenan subunit (K+ salt)

$${m}_{c a r r} = {m}_{IS} * \frac{M {w}_{c a r r}}{M {w}_{IS}} * \frac{n {H}_{IS}}{n {H}_{c a r r}} * \frac{A ( x )}{A ( IS )}$$

The recovery can now be calculated be summing the calculated mass of all carrageenan components and comparing to the mass of the sample. 1 H NMR spectra are manually integrated using Bruker TopSpin 3.6 software (Bruker Biospin, Rheinstetten, Germany)

### REFERENCES

Dyrby, M., Petersen, R. V, Larsen, J., Rudolf, B., Norgaard, L., & Engelsen, S. B. (2004). Towards on-line monitoring of the composition of commercial carrageenan powders. Carbohydrate Polymers, 57(3), 337-348.
Rundlöf, T., Mathiasson, M., Bekiroglu, S., Hakkarainen, B., Bowden, T., & Arvidsson, T. (2010). Survey and qualification of internal standards for quantification by 1H NMR spectroscopy. J Pharm Biomed Anal, 52(5), 645-651. doi:10.1016/j.jpba.2010.02.007
F. van de Velde, S. H.Knutsen, A. I. Usov, H. S. Rollema, A. S. Cerezo. Trends in Food Science & Technology 13 (2002) 73-92

### Determination of Weight Average Molecular Weight (Determination Process 2)

The present method may be used to determine the weight average molecular weight (as well as radius of gyration and polydispersity, if required) of carrageenan. In the present method carrageenan is dissolved in running buffer and analysed by Gel Permeation Chromatography equipped with a Multi Angle Light Scattering detector and a RI detector
Reagents: 0.05 M LiNO₃ with 200ppm N₃⁻: 6.89 g LiNO₃ p.a. and 0.624 g NaN₃ is dissolved in 2.00 L Millipore Water. The eluent is filtered through a 0.22 μm filter.
Control samples: Dextrans can be used to check performance. A small pullulan (Mw<20 kD) with narrow distribution can be used to check alignment and normalisation.
Apparatus: HPLC pump which can deliver a constant flow up to 1 mL/min.
   GPC Columns, e.g. PSS SUPREMA-LUX 1000 Å and PSS SUPREMA-LUX 3000 Å.
   Column oven.
   MALS detector, DAWN EOS from Wyatt Technology Corporation
   RI detector, e.g. Optilab rEX from Wyatt Technology Corporation
Procedure: 10-20 mg sample is weighed out in 10 mL measuring flask. Add a magnet and 7 mL running buffer. Stir for 2 hours.
Samples are filtered through 0.45 μm filter (e.g. 13 mm GHP 0.45 μm Minispike from Waters).
Samples are run on GPC system.
Normal running conditions are:
Flow: 0.6 mL/min; Injection volume: 100μL, Column temperature: 40° C.; Detector temperature:
40° C.; Running time: 2 hours
Note: 1-2 mg/mL is appropriate for most carrageenan with MW from 50 to 1000 kD. The carrageenan may require need heating to dissolve. Filters can shed particles and the first few drops should be discharged.
Results: Weight average molecular weight is calculated using Astra software from Wyatt Technology Corporation.

### Literature

Corredig, M.; Kerr, W.; Wicker, L. “Molecular characterization of commercial pectins by separation with linear mix gel permeation columns in-line with multi-angle light scattering detection.” Food Hydrocolloids 14 (2000) 41-47.
http://www viscotek.com/chromatogram24.php4
Burke, M. D.; Park, J. O.; Srinivasarao, M.; Khan, S. A. “Diffusion of Macromolecules in polymer Solution and Gels: A Laser Scanning Confocal Microscopy study.” Macromolecules, 33 (2000) 7500-7507 Mackier, W., Noy, R.; Sellen, D.B. “Solution Proporties of Sodium Alginate” Biopolymers, 19 (1980) 1839-1860.

### Determination of Acid Insoluble Material (Determination Process 3)

Weigh out 2 g of material, add 135 ml deionized water and 15 ml of 10% sulfuric acid into a blue capped DURAN bottle. Boil for 5 hours. Add 0.5 g of diatomaceous earth and boil for a further 1 hour. Filter the solution into a pre-weighed and dried glass container. The container is then dried in an oven at 105° C., and then cooled in a desiccator and weighed after one hour.

A control value is taken from a new glass container as obtained from the average of triple determination, where the procedure is the same as above minus the sample.

$$The calculation$$

$$\frac{B - A - ( C \times F ) \times 100}{W}$$

A=weight of the dried glass container in grams
B=weight of the dried glass contained+diatomaceous earth+test sample
C=weight of the diatomaceous earth in grams
W=weight of the sample in grams
F=Control factor (see below)

The control factor is obtained from a triple determination of the following:

$$Control factor = \frac{( D - E )}{K}$$

D=weight of dried glass container+diatomaceous earth after filtering and drying in grams
E=weight of dried glass container in grams
K=weight of diatomaceous earth in grams

The control factor is taken as the average of triple determinations and is used to calculate the percentage of acid insoluble material until a new glass container is used.
Sources
FAO, Food and Nutrition
FCC 8th, edition, I suppl.(2012)
   Rheology analysis—Used for example 6

A controlled stress rheometer of the Anton Paar MCR type was used to perform oscillatory strain sweep covering the strain range of 0.001 to 1000%, carried out at a temperature of 5° C. with a parallel plate/plate geometry (PP25/P2) and a gap set at and held constant at 1 mm. To mitigate drying of the sample under analysis water was placed around the outside of the sample. The calculations drawn from this testing were the critical linear viscoelastic region as a function of shear strain, shear stress and storage modulus given in %, Pa, and Pa respectively. The phase angle [°] was reported at a strain of 0.1%, yield point determination was performed from the strain sweep data between points 2 and 37, and finally the consistency index and flow index values were determined from the Power Law model run between points 2 to a data point higher than that corresponding to the yield point. The Power Law model is run automatically from the controlling software package, but briefly can be expressed as follows:

η=k\dot{γ}ⁿ⁻¹

Where K is the consistency index with units of Pasⁿ, and n is the power law index which is dimensionless.

### Rheology Analysis—Used for Example 12

The ice cream samples, stored at 5° C. prior to measurement, are then measured at a constant temperature of 5° C. +/−0.2° C. on an Anton Paar MCR 302 Controlled Stress Rheometer. The measuring geometry is a concentric cylinder CC 27 with corresponding bob probe such that the gap is 3 mm. The viscosity is recorded as a function of a 1-100-1 s−1 shear ramp in a logarithmic distribution, where 25 data points are gathered.

### Texture Analysis—Used for Examples 6 & 7

A Texture Analyser from Stable Microsystems TA-XT type was used to measure the texture attributes of the mousse desserts where the instrument was set up for penetration testing utilizing a P/0.5 probe. The measurement conditions were as follows: pre-test, test, and post-test speeds were identical, 2 mms−1, the penetration depth was set to 15 mm with a trigger force of 2 g. All testing was done at 5° C.

### Texture Analysis—Used for Examples 8, 9 & 10

A Texture Analyser from Stable Microsystems TA-XT type was used to measure the texture attributes of the meat alternative emulsions where the instrument was set up for penetration testing utilizing an SMS P/10 probe.

The measurement conditions were as follows: pre-test, and test speeds were identical, 2 mms−1, while the post-test speed was 10 mm−1 the penetration depth was set to 15 mm with a trigger force of 5 g.

All testing was done at 5° C.

For the meat alternative pieces, which were cut out in 25 mm sausage-like pieces, the similar type of analysis was undertaken. Here, the probe was of the SMS P/20 type, where the penetration depth was set to 2.5mm and the trigger force was 50 g. Pre-test, test, and post-test speeds were as reported for the studies on the emulsion systems above. Temperature of testing was also taken at 5° C.

### Sausage Texture Analysis for Break Strength—Used for Example 11

The texture analysis of the sausage samples is carried out using a Stable Microsystems TA XT plus texture analyser with a flat-bottomed probe of 30 mm diameter where the sausages are measured both hot and cold. Hot temperatures are at 70° C. and cold 5° C. Each sample is tempered at then immediately measured to minimize significant temperature fluctuation. The test is run in compression mode, and continued to fatality of the sausage, such that the force required to break the sausage is recorded and given as the ‘Break Strength’. Compression speed is 1 mm per second.

### Materials

### Food Ingredient Preparation

A mix of Kappaphycus alvarezii and Kappaphycus striatum was freshly harvested and submerged in a salt brine for approximately 1 day. The seaweed was then taken out of the brine, spread on a table and covered for approximately 2 days. The cover was removed and the seaweed was dried to the air for approximately 2 days. A final moisture content of about 24 wt. % was reached.

The dried seaweed was then washed with water at about 15° C. for 20 minutes to remove surface salt, sand and impurities. The washed seaweed was then pasteurized in salt brine at elevated temperatures. The pasteurized seaweed was separated from the salt solution and drained off from excess solution. The material was then dried on trays placed in a fan assisted oven at 65° C. until reaching constant final dry weight. The dried seaweed was then milled into a seaweed flour.

Analysis by ¹H-NMR spectroscopy indicated the carrageenan profile was composed of 77.5% of kappa, 11.8% of mu, 8.7% of iota, 1.1% of nu, and 0.9% of lambda type.

### Carrageenan Content

The samples were analysed in accordance with the phycocolloid profiling method described herein and the results are provided in table 1.

TABLE 1
¹H NMR analysis of several Seaweed Flours compared to (semi-)refined products
both based on a combination of seaweed of the species Kappaphycus striatus and
Kappaphycus alvarezii (known commercially as cottonii type seaweed).

| | | | | | | | | | Recovery |
|---|---|---|---|---|---|---|---|---|---|
| Product | Lambda | Nu | Starch | Iota | Theta | Mu | Kappa | Beta | (%) |
| Sample 1.5 | ND | 0.4 | 0.2 | 6.2 | ND | 9.2 | 51.4 | ND | 67.5 |
| Sample 25.5a | ND | 1.3 | ND | 5.3 | ND | 12.5 | 50.3 | ND | 69.5 |
| Sample 38.1 | ND | 1.0 | ND | 7.8 | ND | 8.2 | 39.6 | 0.8 | 57.4 |
| Sample 38.2 | ND | 1.1 | ND | 7.0 | ND | 9.4 | 36.9 | 0.7 | 55. |
| Sample | ND | ND | ND | 5.2 | ND | 5.6 | 43.0 | ND | 53.9 |
| P2-2020028 | | | | | | | | | |
| Sample | ND | ND | ND | 4.7 | ND | 5.0 | 40.7 | ND | 50.4 |
| P3-2020029 | | | | | | | | | |
| Semi-refined | ND | ND | 0.5 | 7.8 | ND | 1.3 | 62.1 | ND | 72.0 |
| kappa | | | | | | | | | |
| carrageenan | | | | | | | | | |
| Refined kappa | ND | ND | 1.2 | 9.1 | ND | 1.6 | 78.0 | ND | 89.1 |
| carrageenan | | | | | | | | | |

The samples of flour were found to vary in amounts of carrageenan based the location and time of year of their collection.

### Molecular Weight

Refined carrageenan based on cottonii type seaweeds (depending on the degree of modification) typically ranges between 200 kDa and 1000 kDa, or more likely in the range of 200-800 kDa.

TABLE 2
HPSEC analysis of several seaweed flours compared to (semi-)refined
kappa carrageenan both based on cottonii type seaweed.

| | Weight Average | |
|---|---|---|
| | Molecular | Polydispersity |
| Sample | weight (kDa) | Index |
| Refined kappa carrageenan | 600 | 2.1 |
| Semi-refined kappa carrageenan | 500 | 2.8 |
| Seaweed flour (sample 25.5a) | 1500 | 1.8 |
| Seaweed flour (sample 38.1) | 910 | 1.3 |
| Seaweed flour (sample 38.2) | 1300 | 1.3 |
| Seaweed flour (sample 38.3) | 900 | 1.8 |
| Seaweed flour (sample P2- | 750 | 2.1 |
| 2020028) | | |
| Seaweed flour (sample P3- | 780 | 2.8 |
| 2020029) | | |

### Sample Preparation and Evaluation

The samples using the method below and based on the following general recipe

| Ingredient type | Dosage |
|---|---|
| Water | Up to 100 |
| Plant protein or Almond paste | 0-4% |
| Sunflower oil | 0-2% |
| Emulsifier | 0-0.15% |
| Cocoa powder or instant coffee | 0-1.2% |
| Sucrose | 0-6% |
| Seaweed flour or E418 or E460, E466, E407 | 0-0.4% |
| Salts | 0-0.5% |
| Flavors | 0-0.2% |

### General Process

1. If proteins are used, start by hydrating these according to recommendations
2. Add and hydrate other dry ingredients while mixing at 60° C.-65° C.
3. Add liquid ingredients

### Without UHT

4. Mix well (e.g. Silverson for 2 minutes at 4000 RPM)
5. Heat at 95° C. for 10 minutes and proceed to step 9.

### With UHT

6. Mix well (e.g. Silverson for 2 minutes at 4000 RPM)
7. Preheat to 90° C. for 30 seconds
8. UHT THE 142° C. for 3 seconds
9. Homogenize at 200 bar, 70° C., or in the case of chocolate milk homogenize the samples with two-step homogenization at 250 bar and 40 bar, 70° C.
10. Cool to 10° C. and fill bottles

### LUMiSizer

All measurements were performed in triplicate.

Samples were redispersed before transfer to the Lumisizer tubes.

Experiments were run at a centrifugation speed of 2325 RCF for 45 minutes at 10° C. with 10 second scanning intervals at a wavelength of 870 nm.

### Rheometer (Flow Curves)

Samples were redispersed before transfer to the rheometer, the measurement was performed at room temperature,

The viscosity was measured in mPa·s at shear rates ranging from 0.1 s⁻¹ to 1000 s⁻¹ with an Anton Paar rheometer using double gap geometry (DG26.7).

### Rheometer (Cooling Curves)

Samples were redispersed before transfer to the rheometer, the sample was then heated to

The storage modulus (G′) and the loss modulus (G″) were recorded at a strain of 1% during cooling from 90° C. to 10° C. at a cooling rate of 1.5° C. per minute in an Anton Paar rheometer using double gap geometry (DG26.7).

### EXAMPLE 1

A beverage of skimmed milk containing chocolate was produced containing sample seaweed flour (sample 1.5) in an amount of 800 ppm (0.08 wt. %). The product was found to be stable.

The product of the present invention was compared with GRINDSTED® Carrageenan CL 220, a product that is commercially used in chocolate milk. This was also found to be stable. Comparison was also made with refined kappa carrageenan.

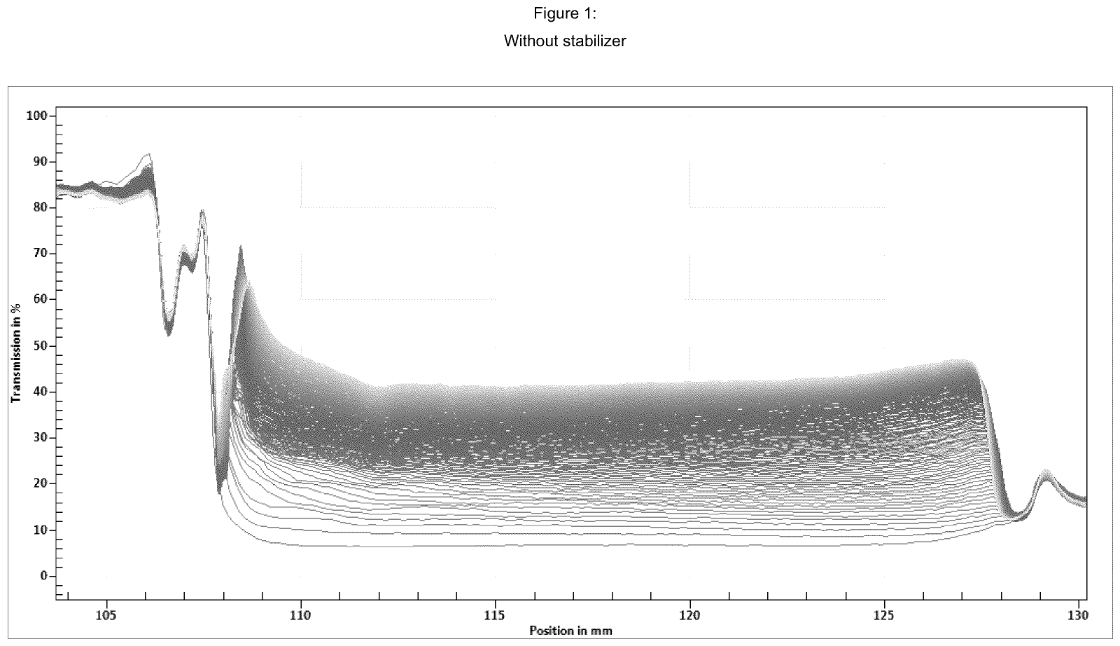

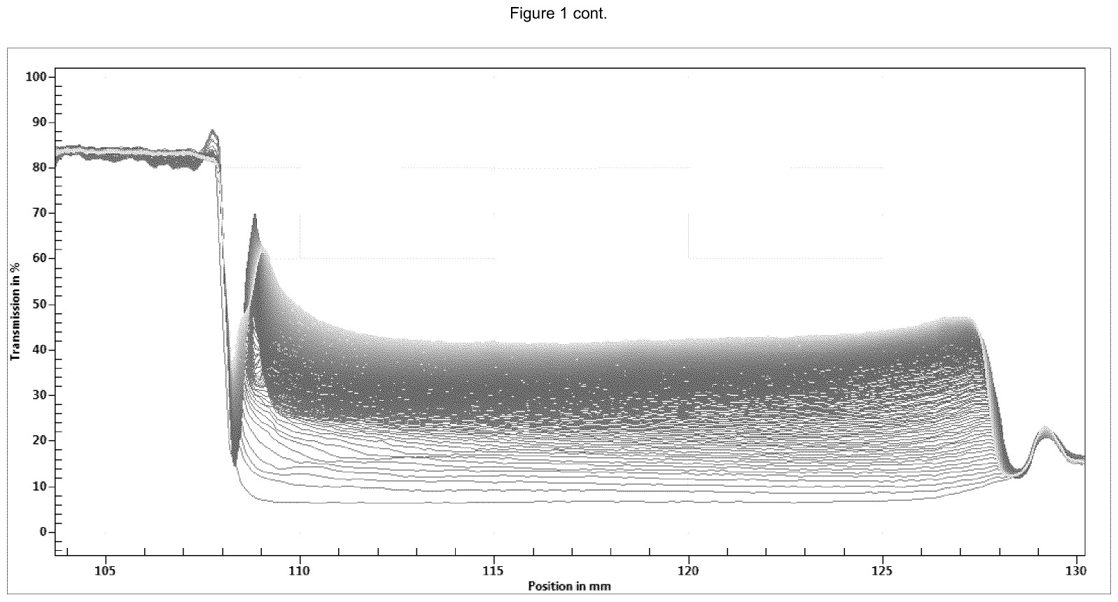

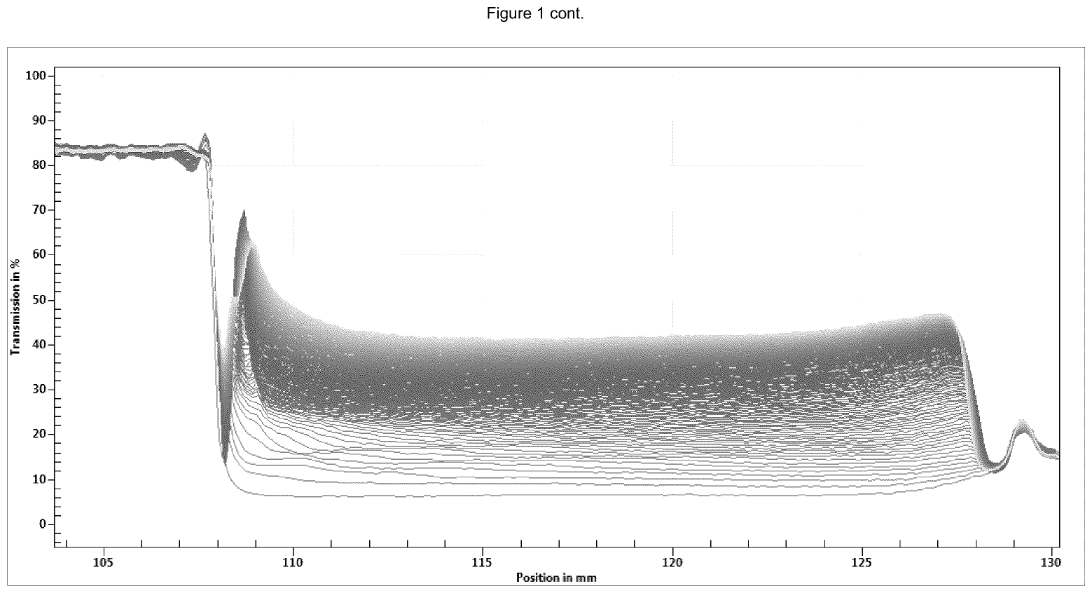

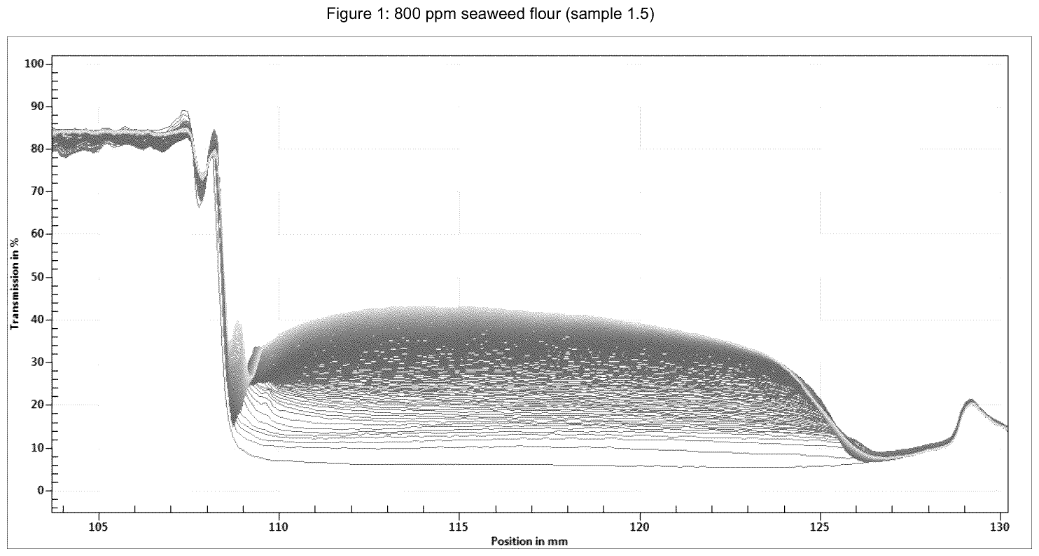

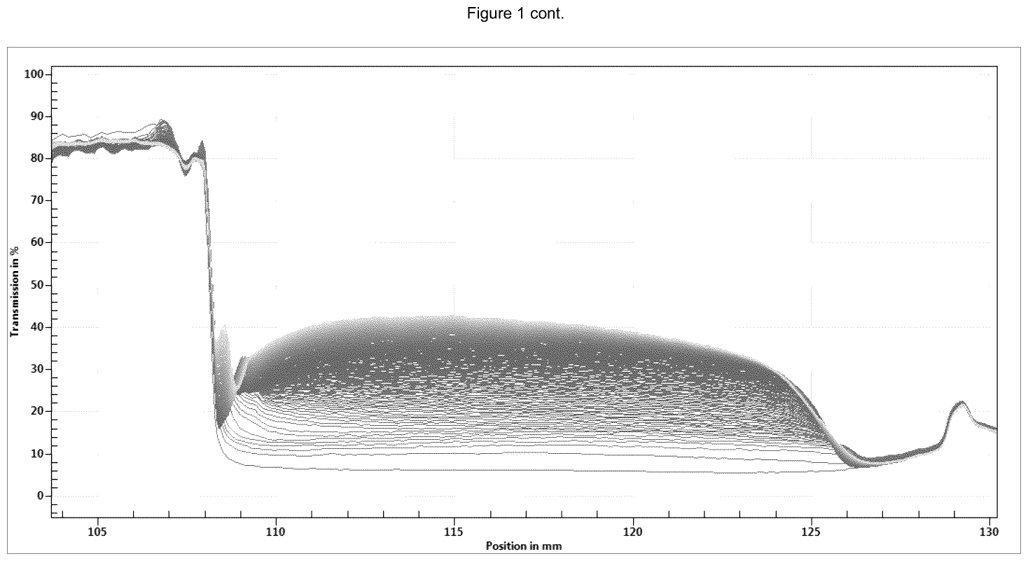

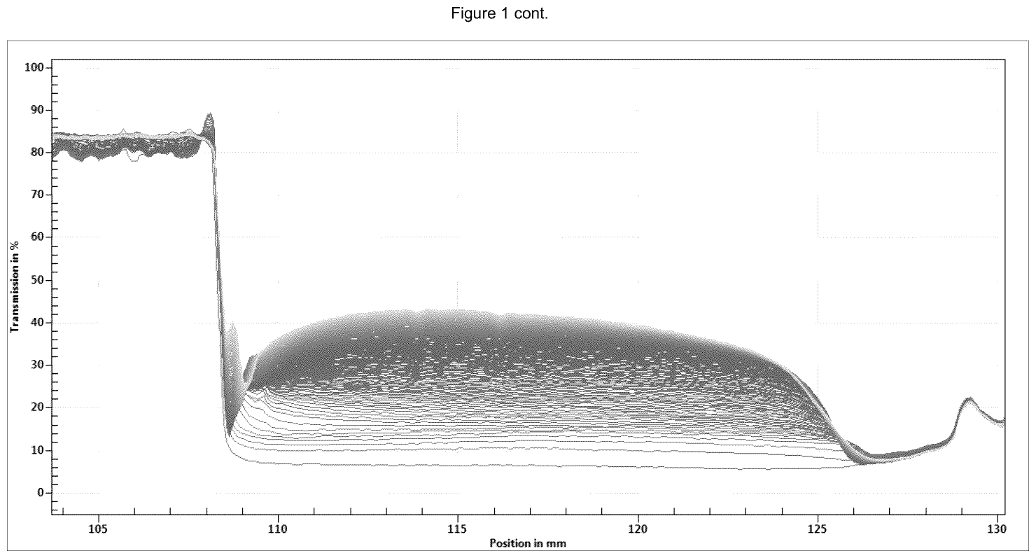

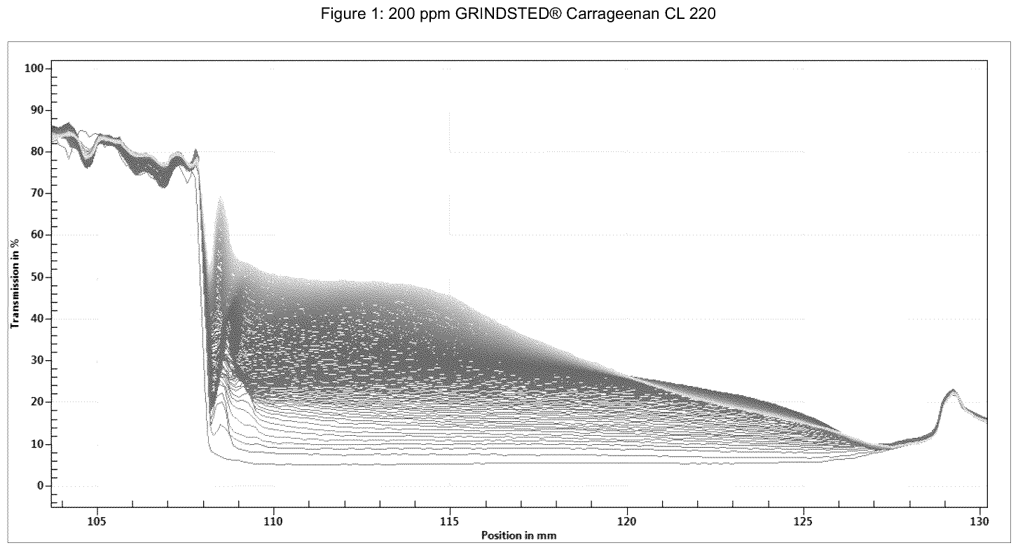

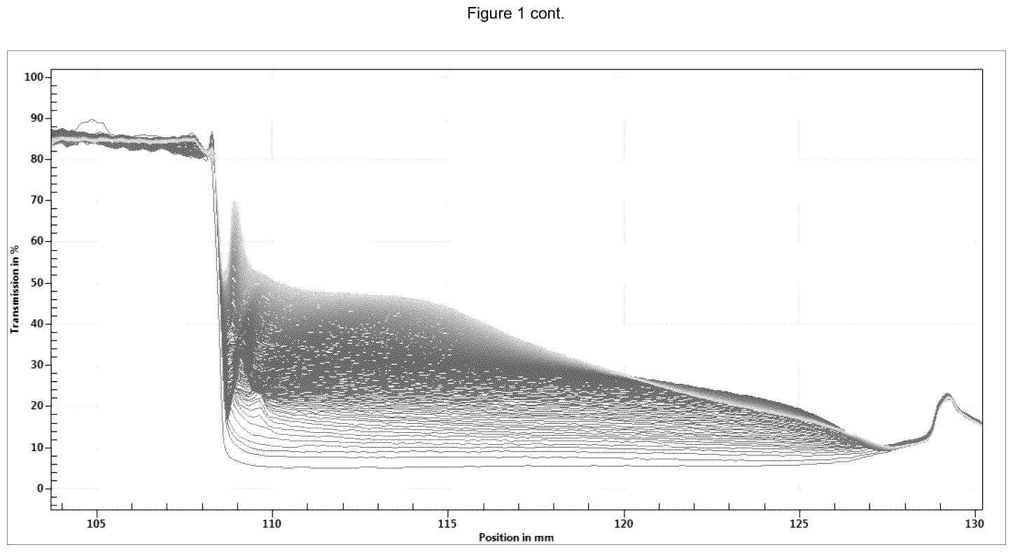

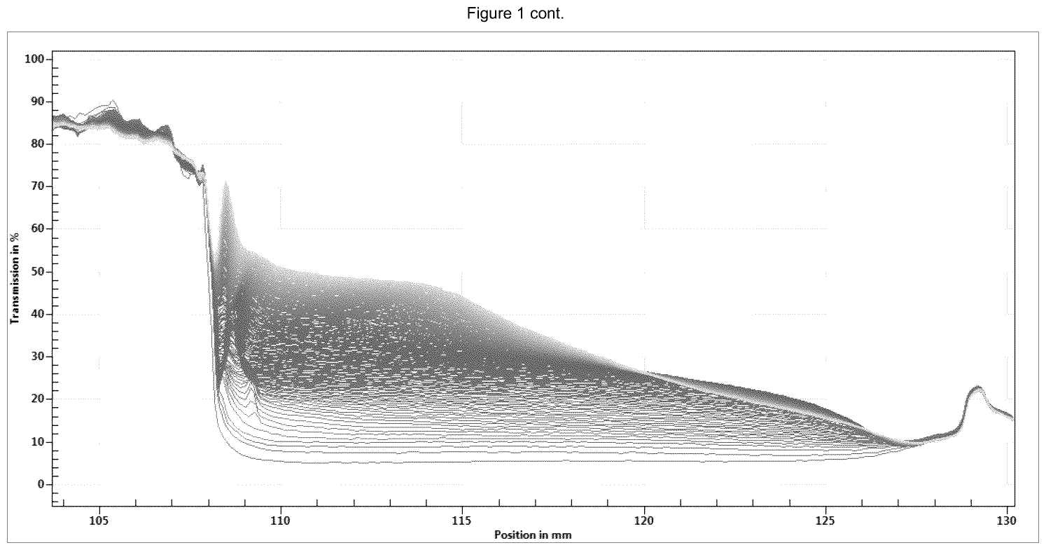

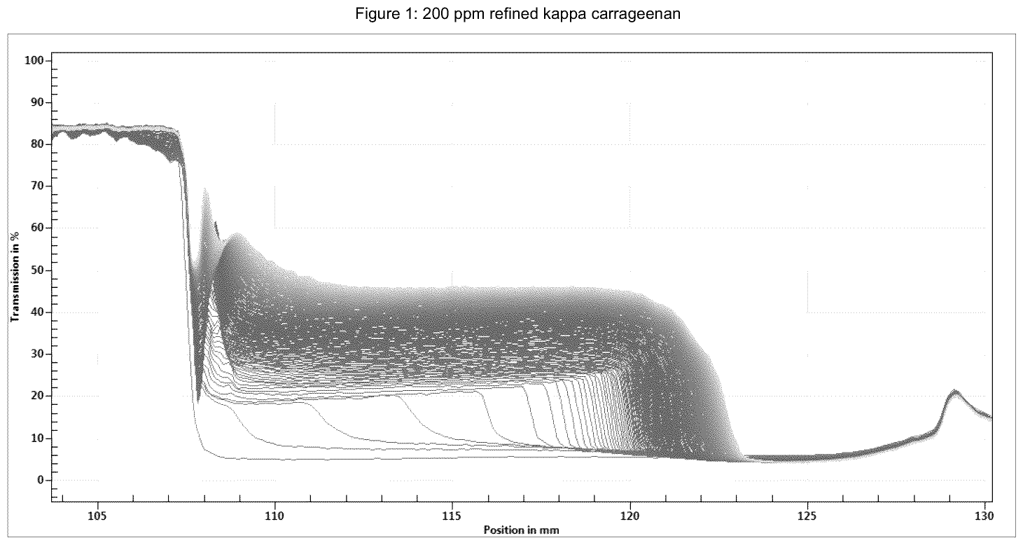

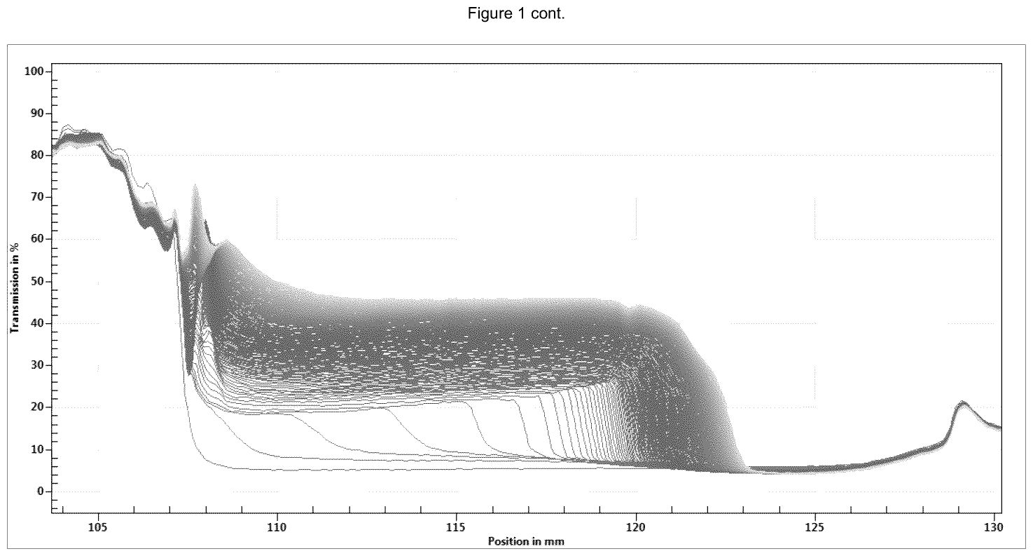

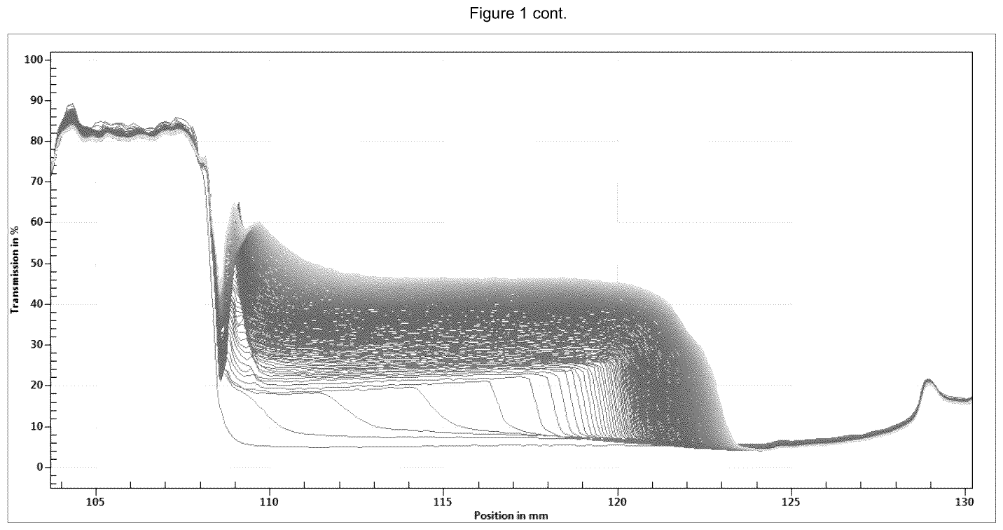

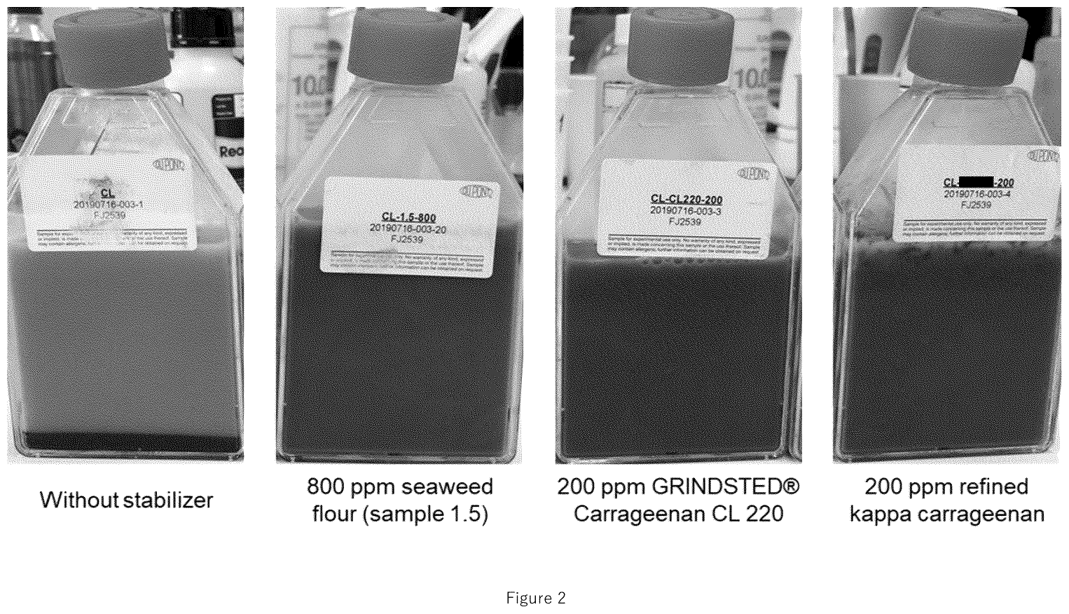

In FIG. 2 you can see that refined kappa carrageenan was found to gel too much and that small gels are formed in the top layer. The LUMiSizer plots in FIG. 1 show that the sediment layer of seaweed flour (sample 1.5) at 800 ppm is about the same size as for GRINDSTED® Carrageenan CL 220 at 200 ppm. The sedimentation layer can be seen on the right size of the graph (which corresponds to the bottom of the centrifuge tube) as non-transparent areas (the higher the response the more transparent the solution is). The size of the cocoa sedimentation layer gives an indication the stabilizing power of the added ingredient. It can be noted that the cocoa sedimentation layer for refined kappa carrageenan is even higher, but the physical sample (FIG. 2) shows that the sample is over stabilized and shows excessive gelling.

### EXAMPLE 2

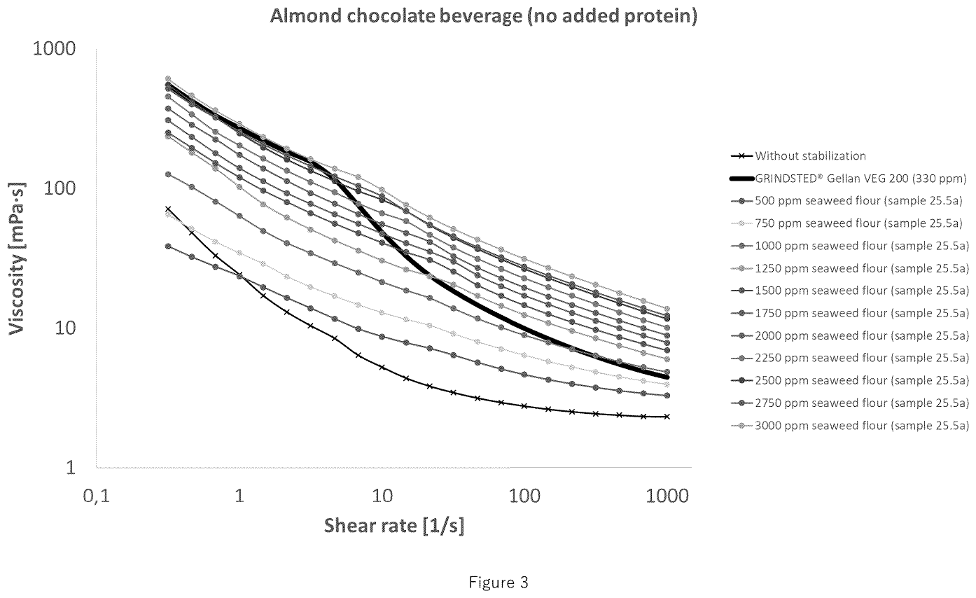

Cocoa is often used to simulate a stability challenge in plant-based beverage model systems. This series compares the functionality of a dosage range of seaweed flour (sample 25.5a) in accordance with the present invention to GRINDSTED® Gellan VEG 200 at 330 ppm (FIG. 3).

Flow curves may be used to predict stability and mouthfeel of beverages. Viscosity retention at higher shear rates (e.g. 100 1/s) can be seen as a precursor for mouthfeel, while higher viscosities at lower shear rates is often used as an indicator for suspension power during shelf-life.

FIG. 3 shows that seaweed flour (sample 25.5a) in accordance with the present invention retains more viscosity at higher shear rates compared to GRINDSTED® Gellan VEG 200 at 330 ppm. While gellan often needs to be supplemented with an extra viscosifier (e.g. LBG or guar) to compensate for the lack of mouthfeel. In contrast, the product of the present invention may be used as a stabilizer and viscosifier at the same time, which will result in a smaller ingredient list on the final product.

### EXAMPLE 3

We have found that the functionality of the food ingredient of the present invention may be further enhanced when it is used in combination with protein concentrates/isolates. This further enhancement is particularly found in plant-based beverages, but is not limited to that application.

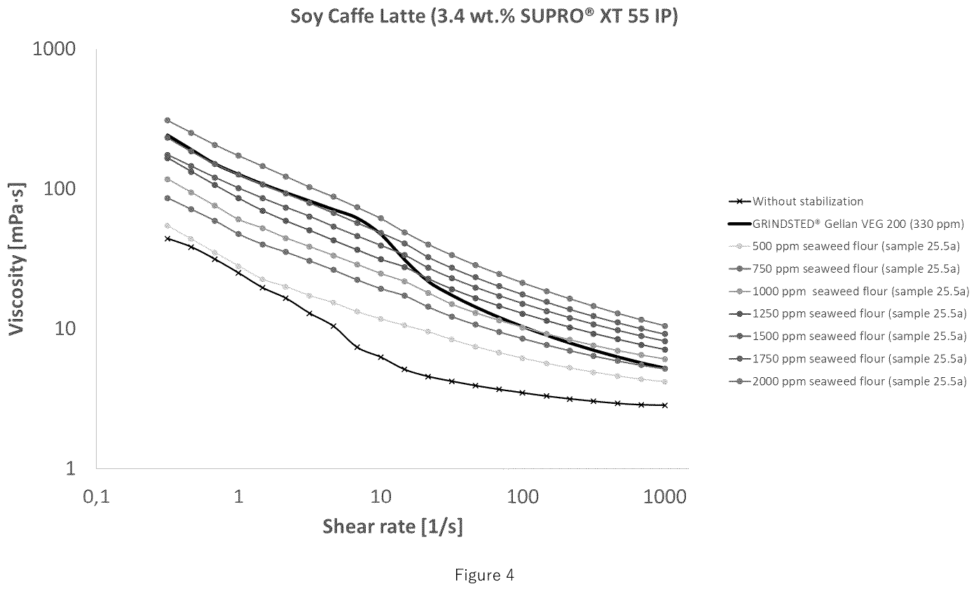

FIG. 4 shows that the presence of soy protein (SUPRO® XT 55 IP) the food ingredient of the present invention (sample 25.5a) need only be dosed at an amount of 1750 ppm to provide a comparable viscosity at a shear rate of 1/s to a dosage of 3000 ppm in the absence of soy protein (see FIG. 3). The present food ingredient at 1750 ppm also provides a significantly higher mouthfeel by providing a higher viscosity at 100/s compared to GRINDSTED® Gellan VEG 200 dosed at 330 ppm.

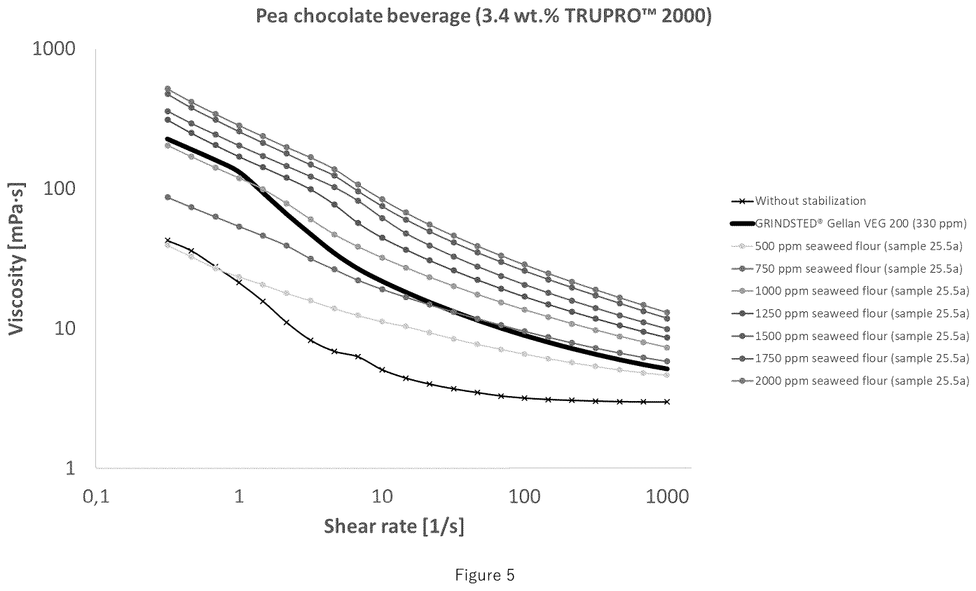

FIG. 5 shows that the presence of pea protein (3.4 wt. % TRUPRO™ 2000) brings the comparable viscosity with GRINDSTEDO Gellan VEG 200 dosed at 330 ppm, at 1/s even further down to a dosage level of the present food ingredient slightly above 1000 ppm.

### EXAMPLE 4

### Galactomannans and Glucomannans

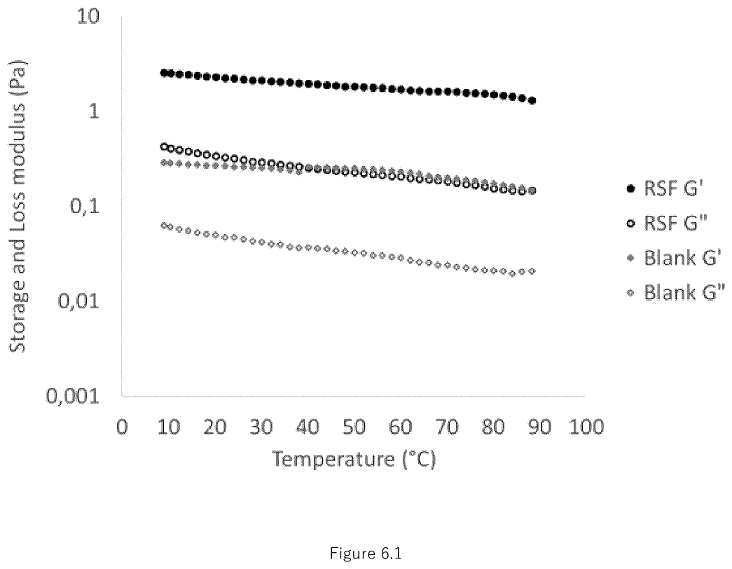

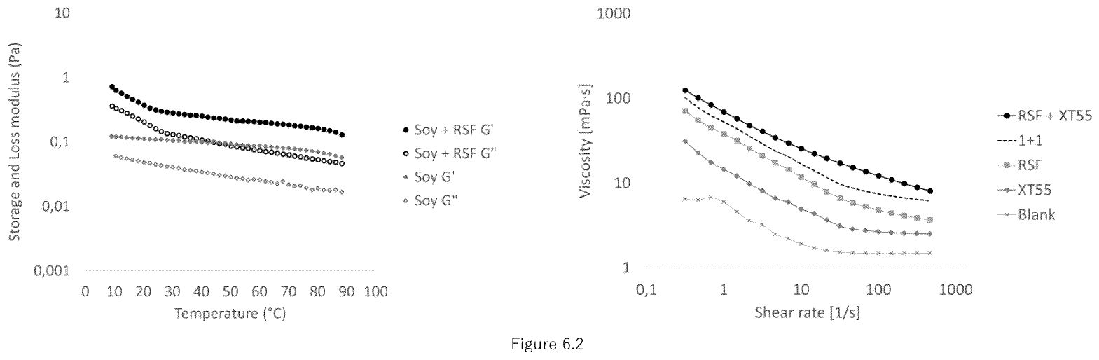

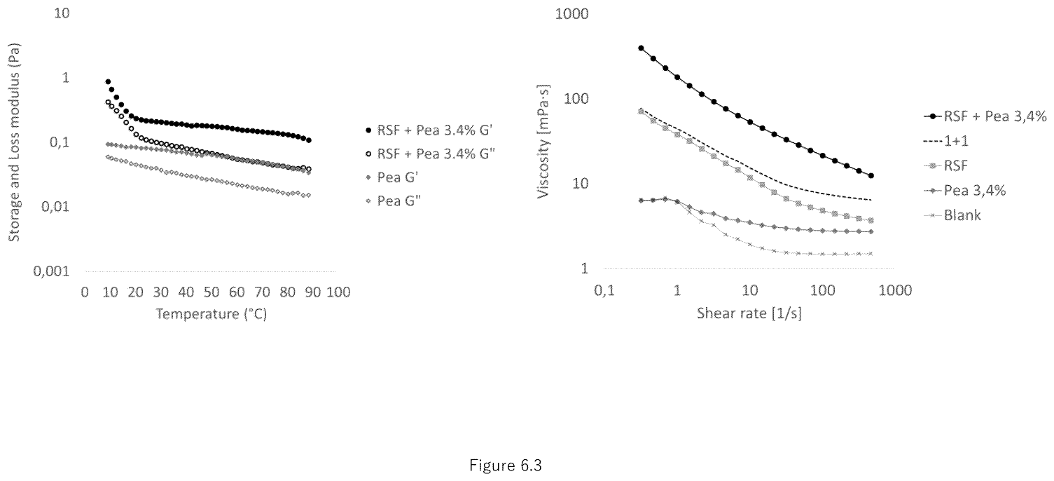

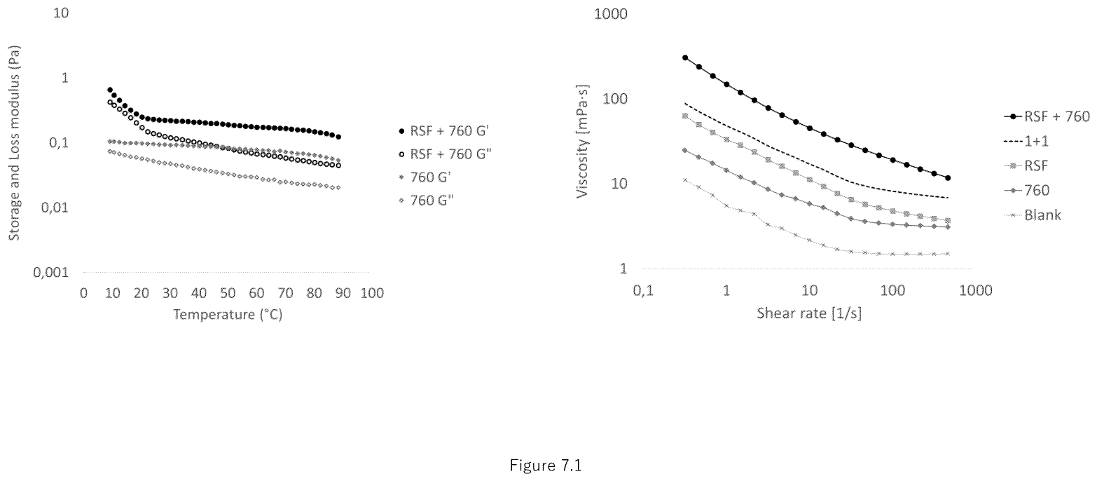

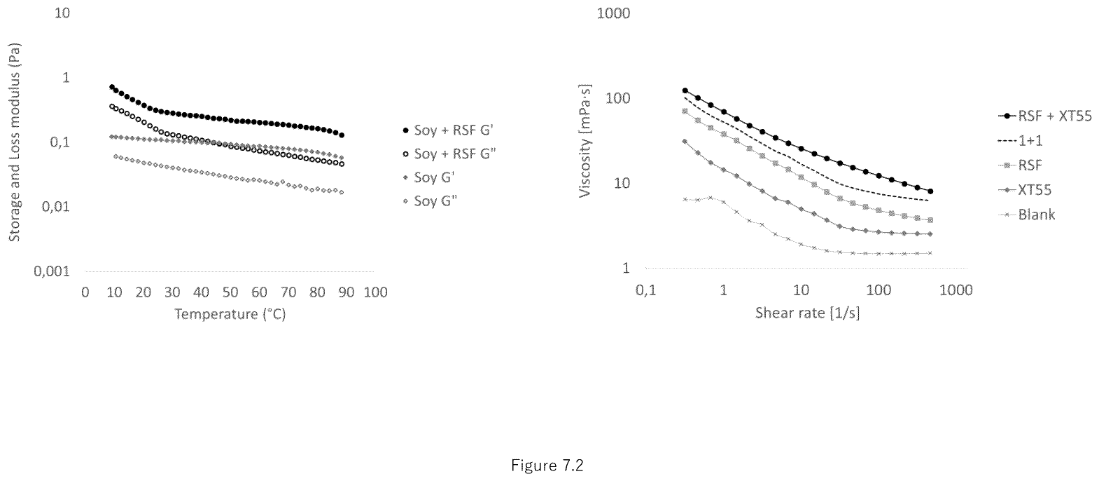

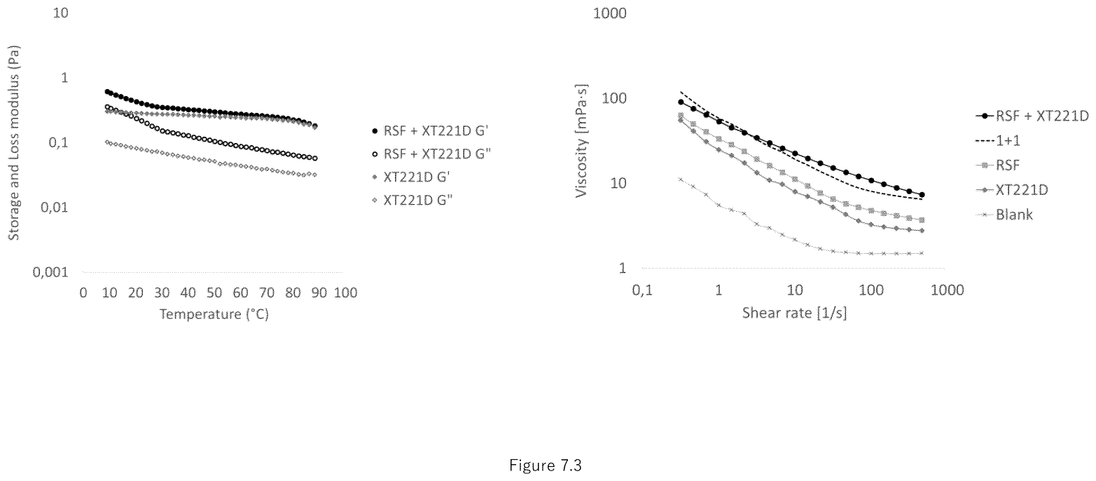

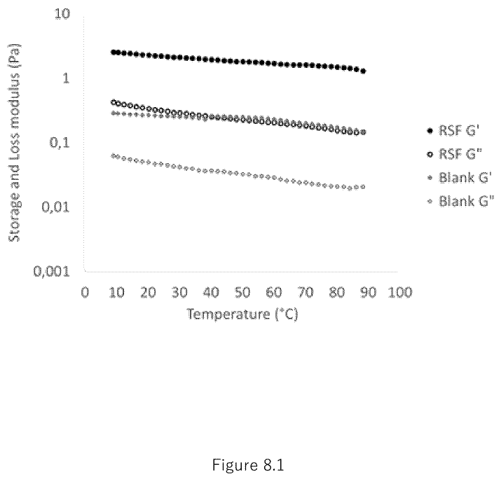

FIGS. 6 and 7 demonstrate that there are significant changes in the cooling curves and in flow curves once seaweed flour (sample 1.5) is combined with pea protein and different types of soy isolates. While seaweed flour (sample 1.5) at a concentration of 1000 ppm does not show a significant shift in G′ and G″ upon cooling, significant changes in both G′ and G″ do occur when pea protein or soy isolate is present. A similar effect is also observable in the flow curves, where a significant increase in viscosity is achieved when seaweed flour (sample 1.5) is combined with pea protein or soy isolate.

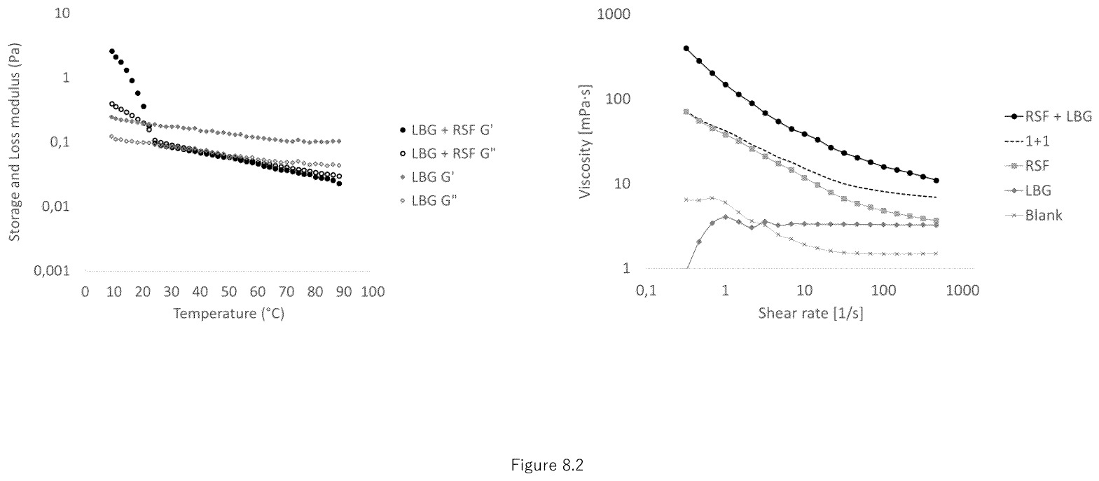

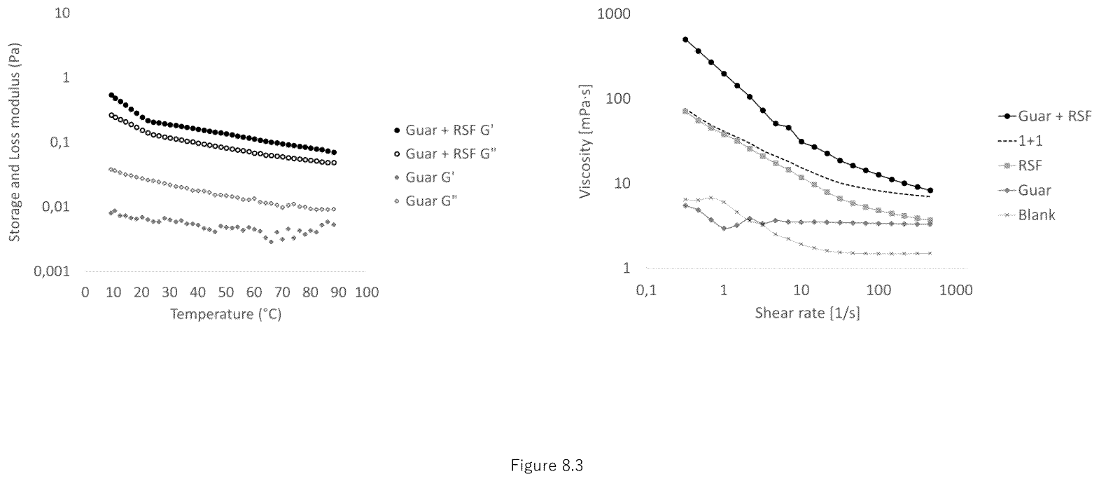

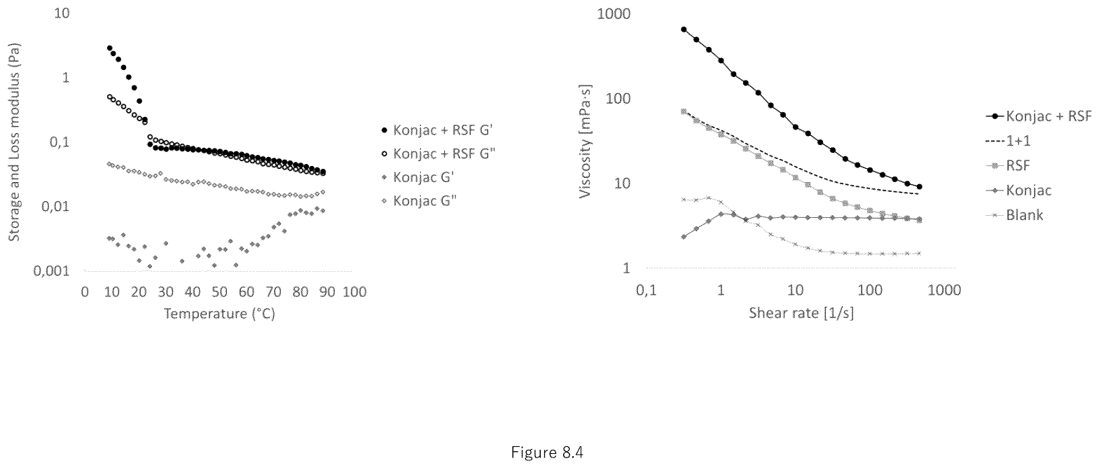

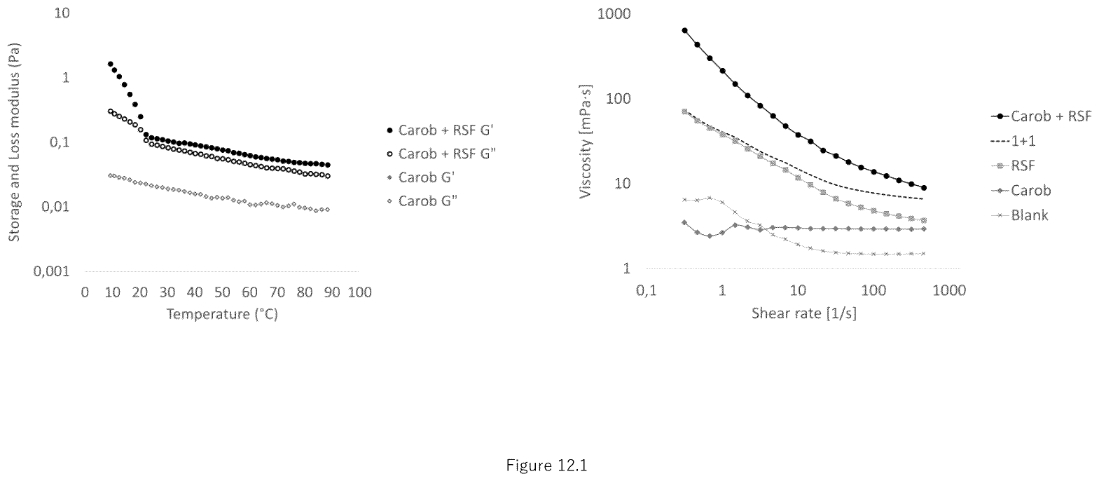

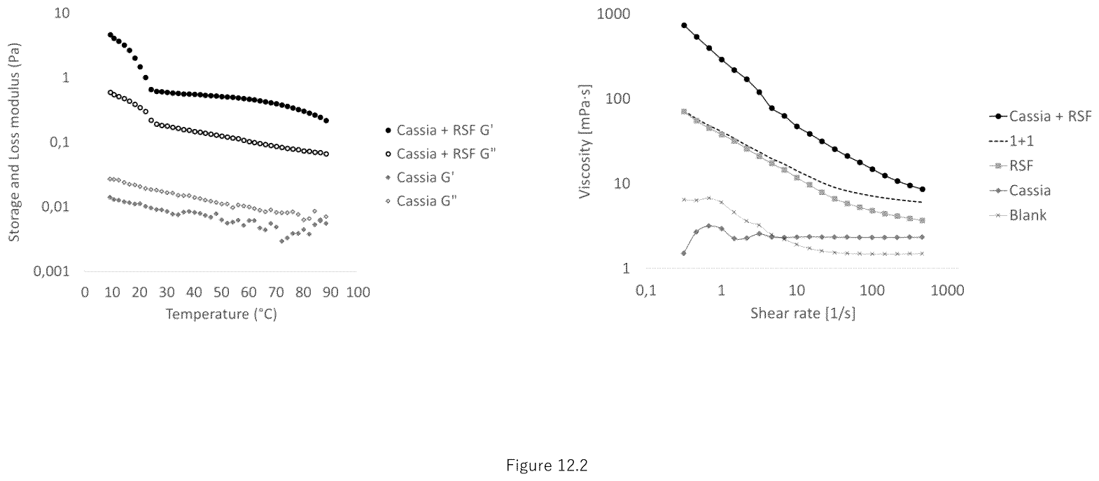

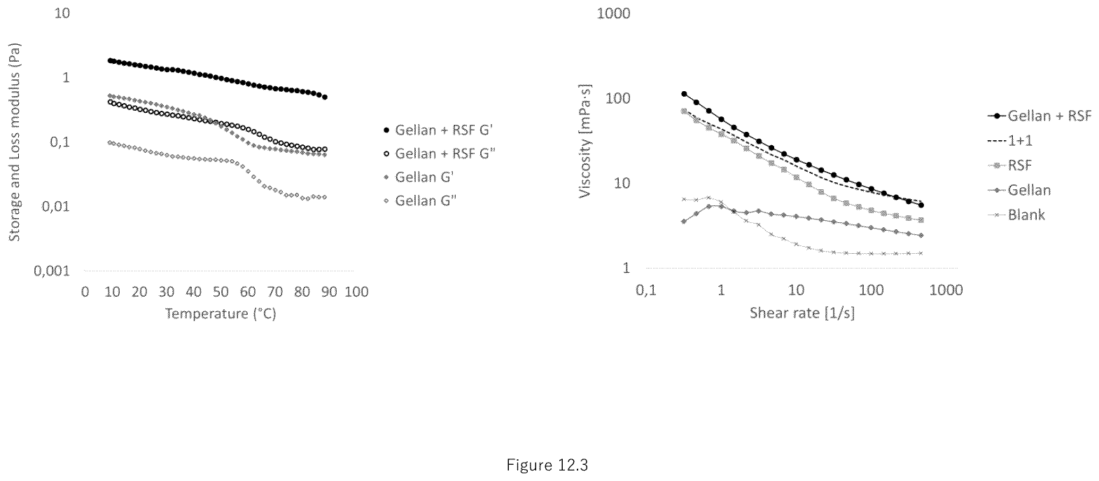

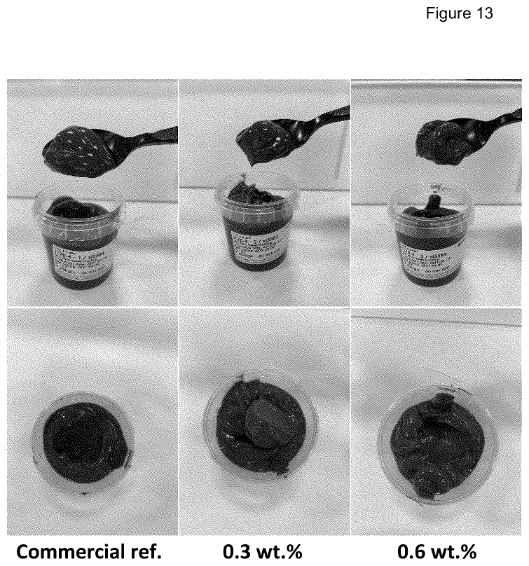

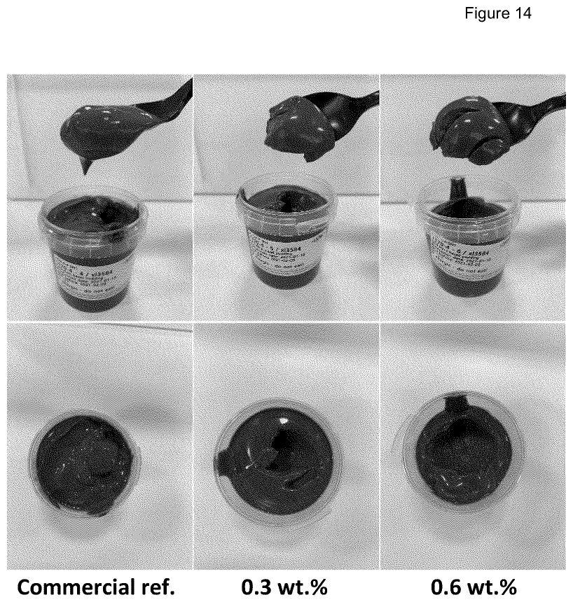

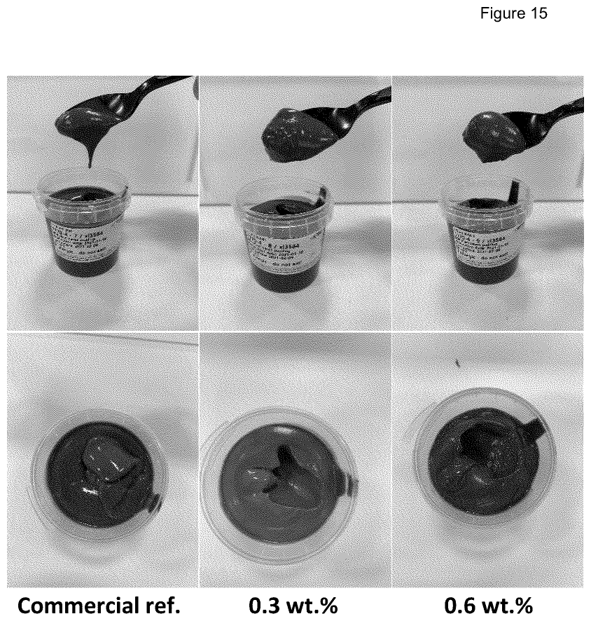

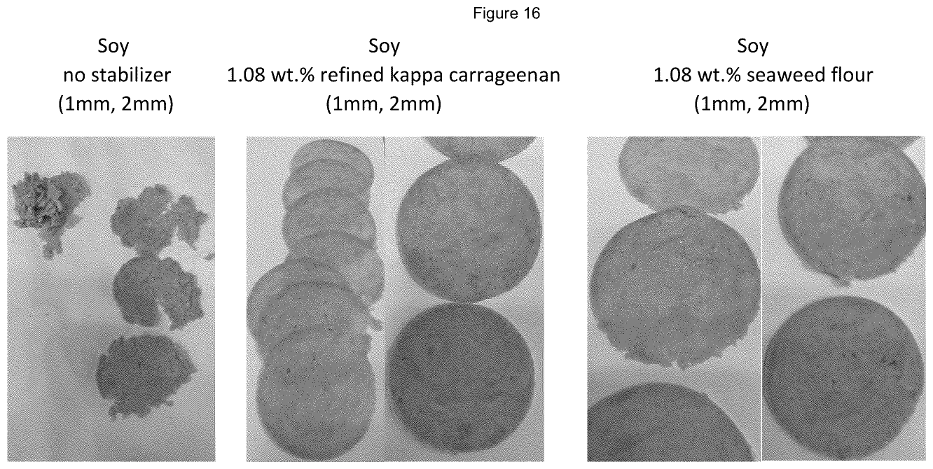

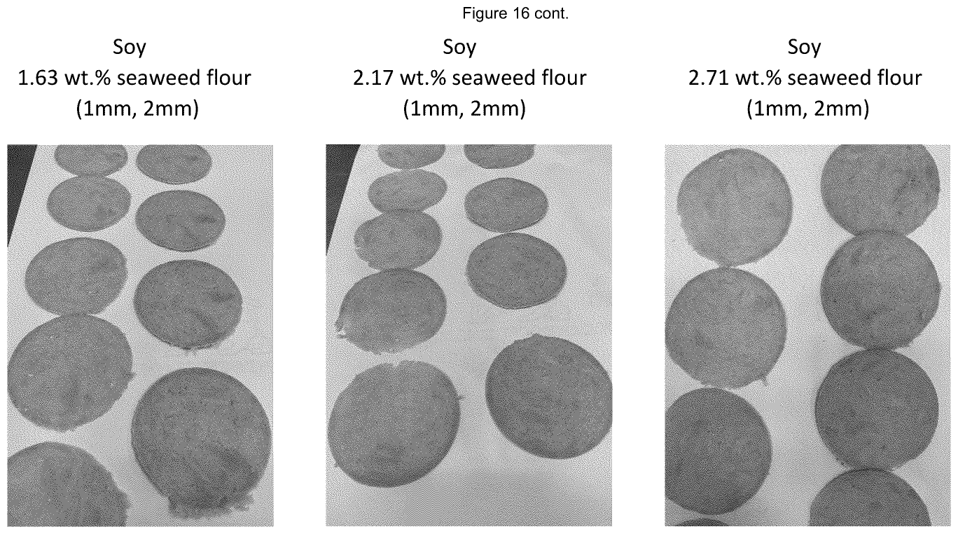

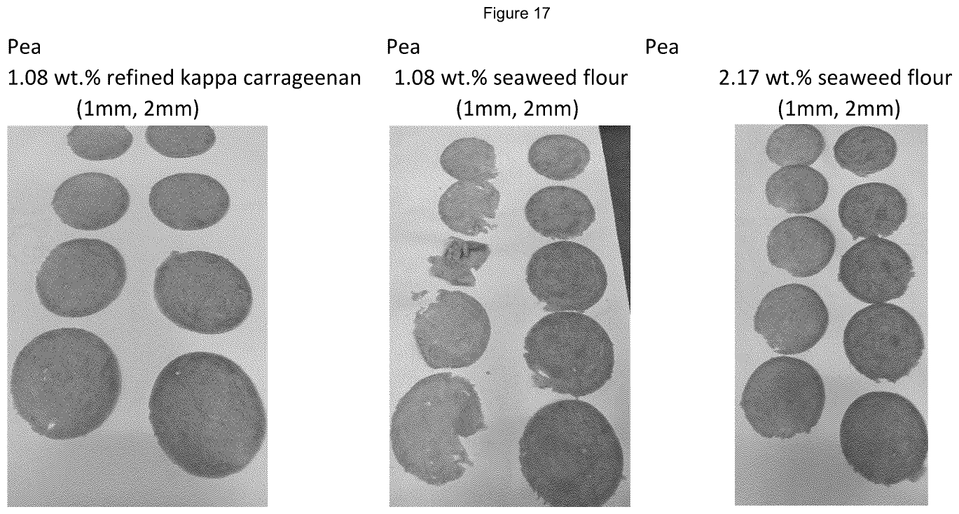

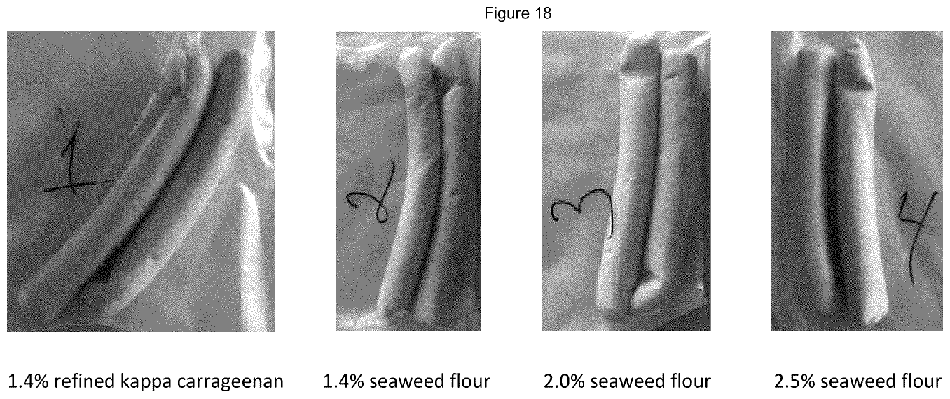

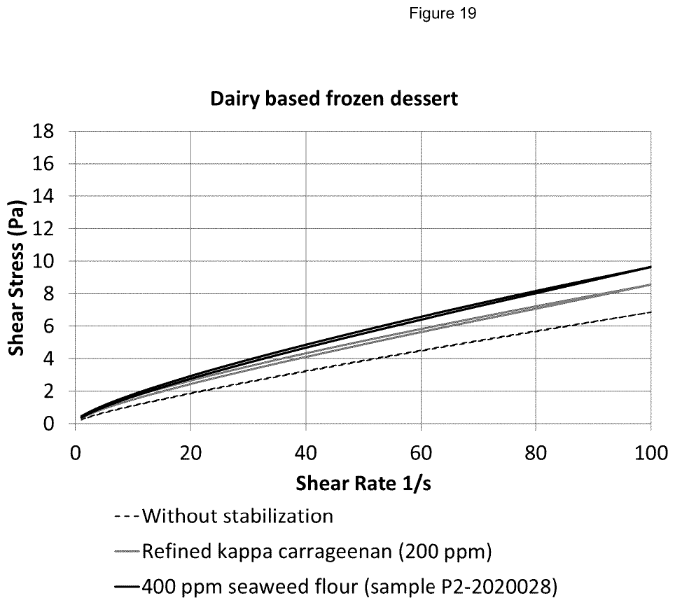

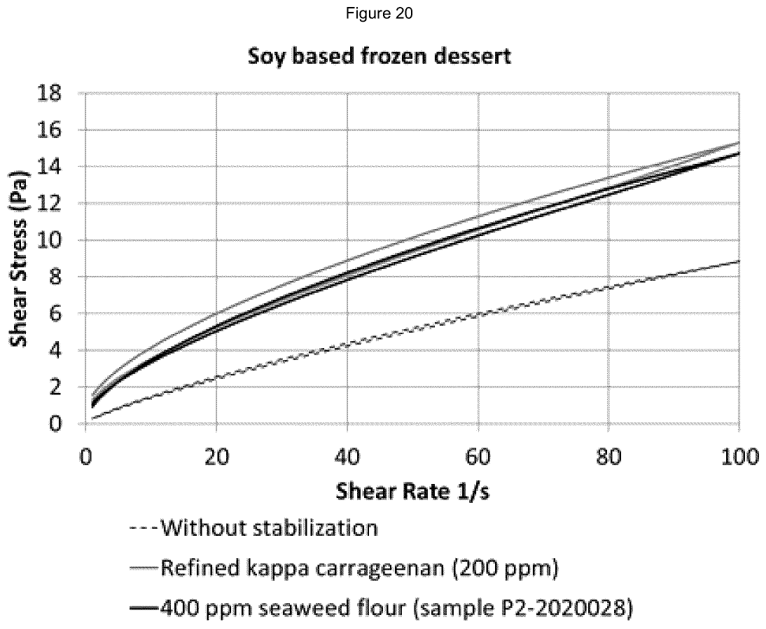

FIGS. 8 and 12 demonstrate in a similar way, that upon combining seaweed flour (sample 1.5) with several galactomannans and glucomannans, similar disproportional increases are found in both cooling curves and flow curve viscosities. In a situation where 1000 ppm of either is not enough to induce significant changes in the cooling and flow curves, the combined effect of both ingredients creates a disproportionally strong response. While there are subtle differences between the different galactomannans and glucomannans used, the shifts in both G′ and G″ upon cooling and the viscosity increases of the combined combinations in the flow curves, indicate clear synergy, that can be used in beverages, but also in other applications.

The change in G′ and G″ upon cooling indicate that both ingredients are interacting at a level that they are not capable on their own (e.g. 1+1=3).

### EXAMPLE 5

Soy drinks are typically stabilized with iota-type carrageenan, carboxymethyl cellulose or gellan. The experiments depicted herein show that seaweed flour based on cottonii type seaweeds is also able to stabilize these types of systems, while this is not an application that would typically use (semi-)refined carrageenan based on cottonii type seaweed.

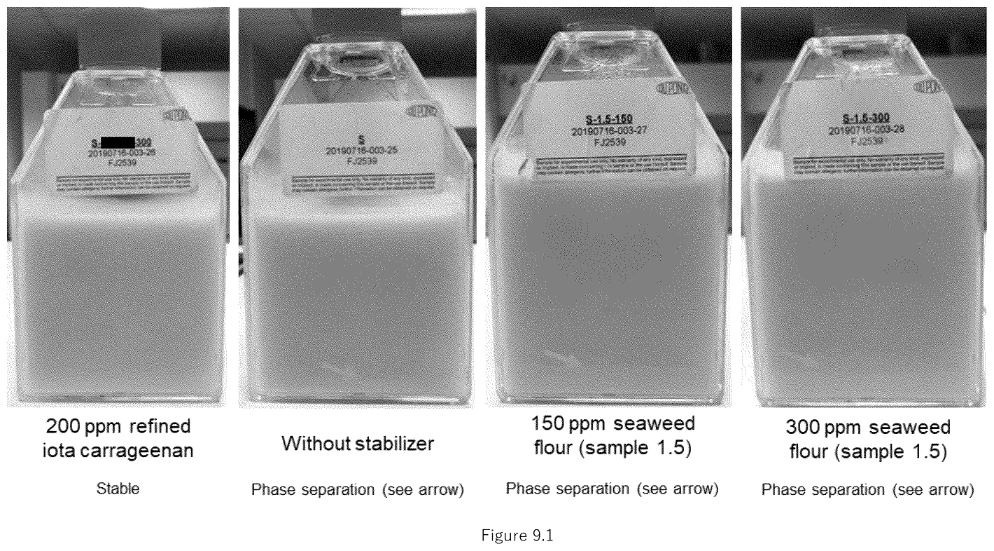

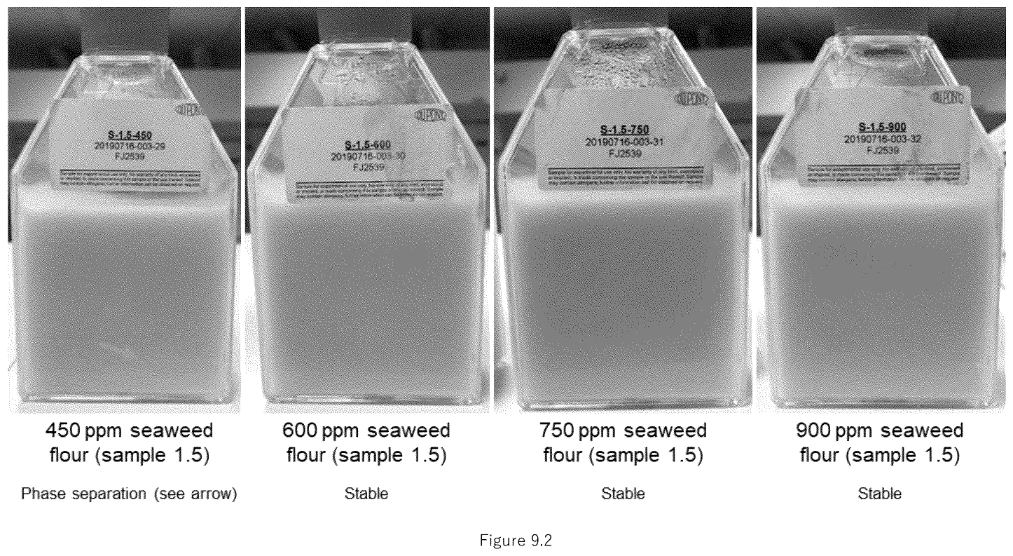

On the visual shelf-life analysis of soy drinks (FIG. 9): Only the refined iota carrageenan control and the seaweed flour (sample 1.5) stabilized samples at 600 ppm, 750 ppm and 900 ppm, showed no phase separation after overnight storage. After three months of refrigerated storage at 4° C., 600 ppm, 750 ppm and 900 ppm did not show phase separation or instability (FIG. 9).

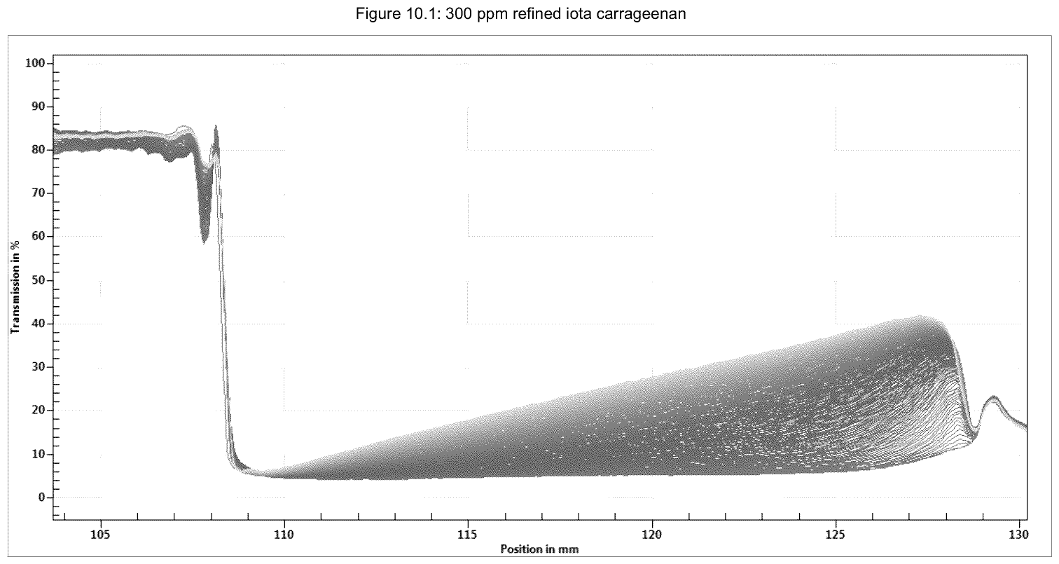

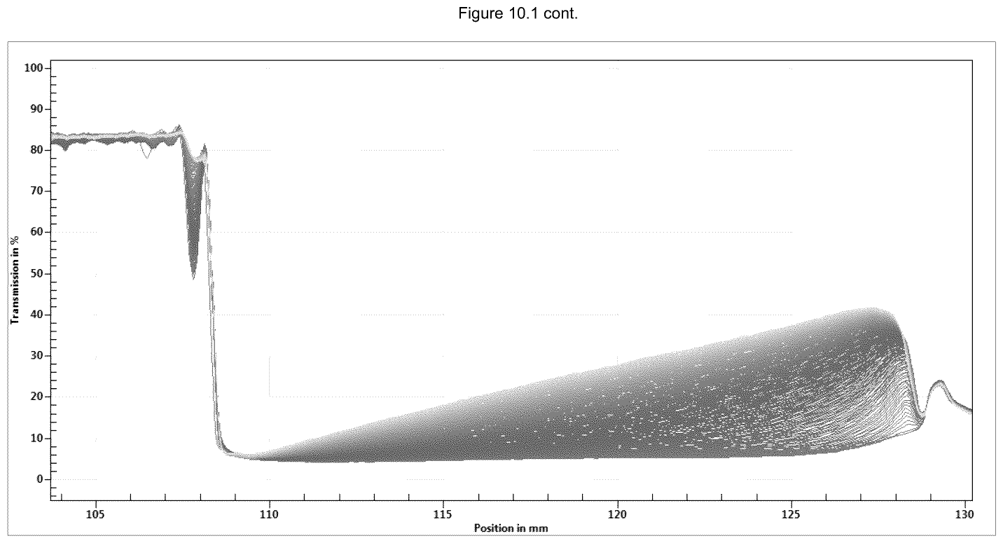

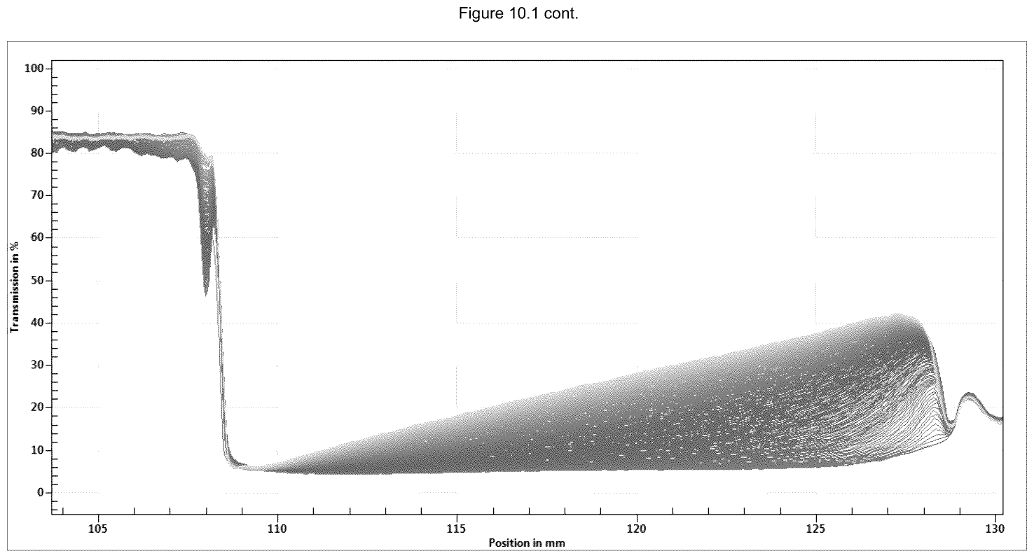

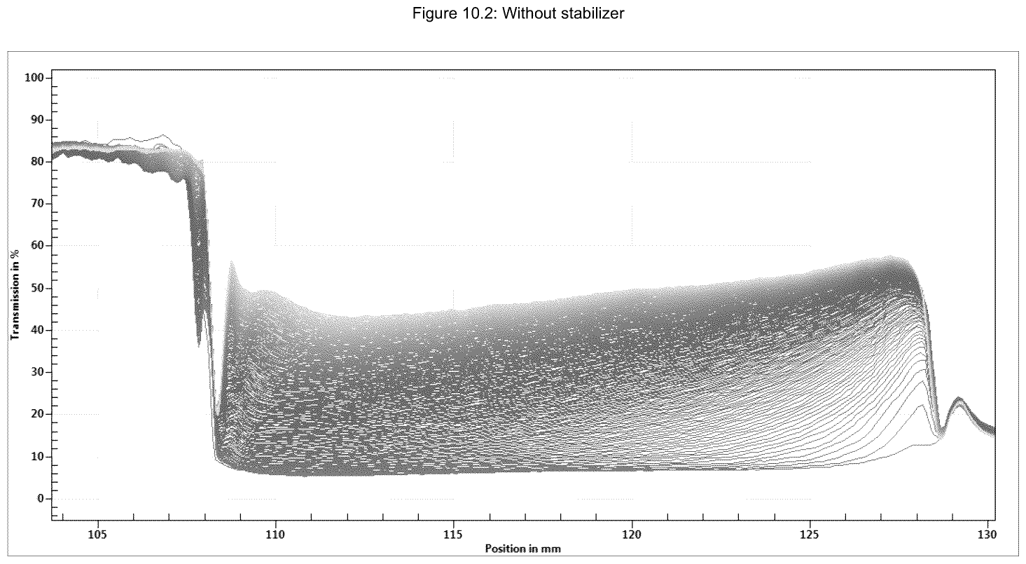

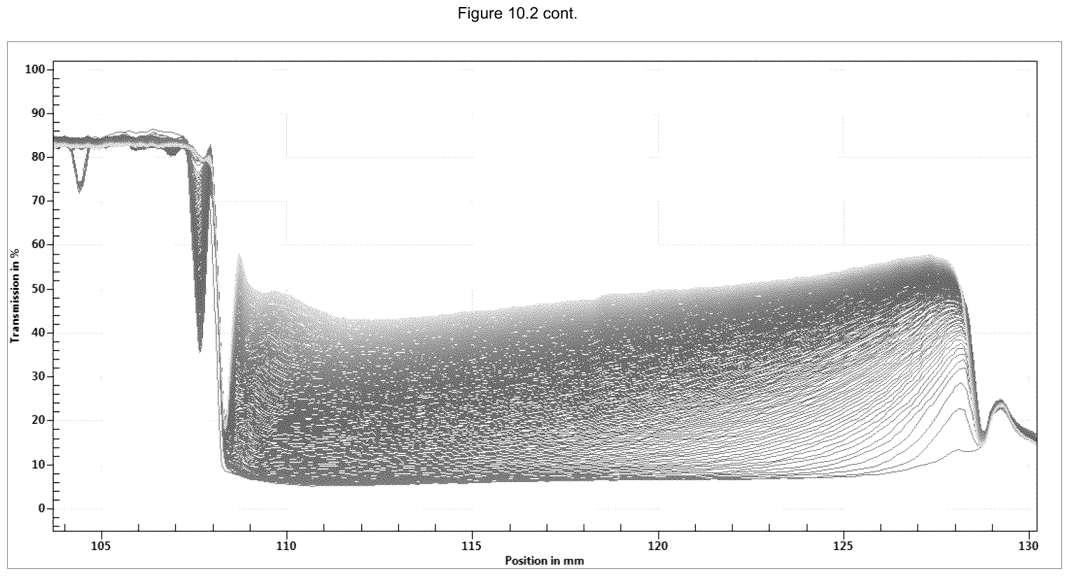

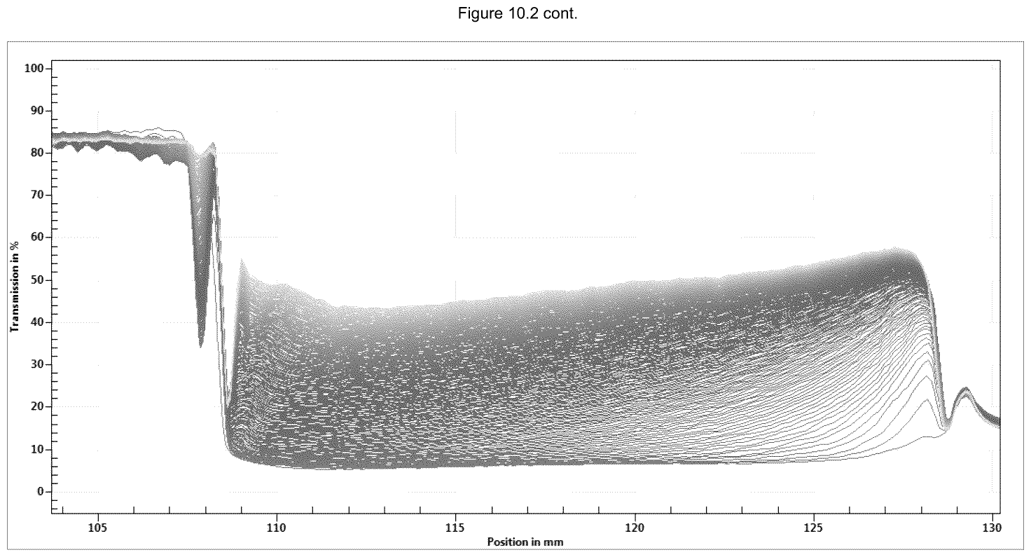

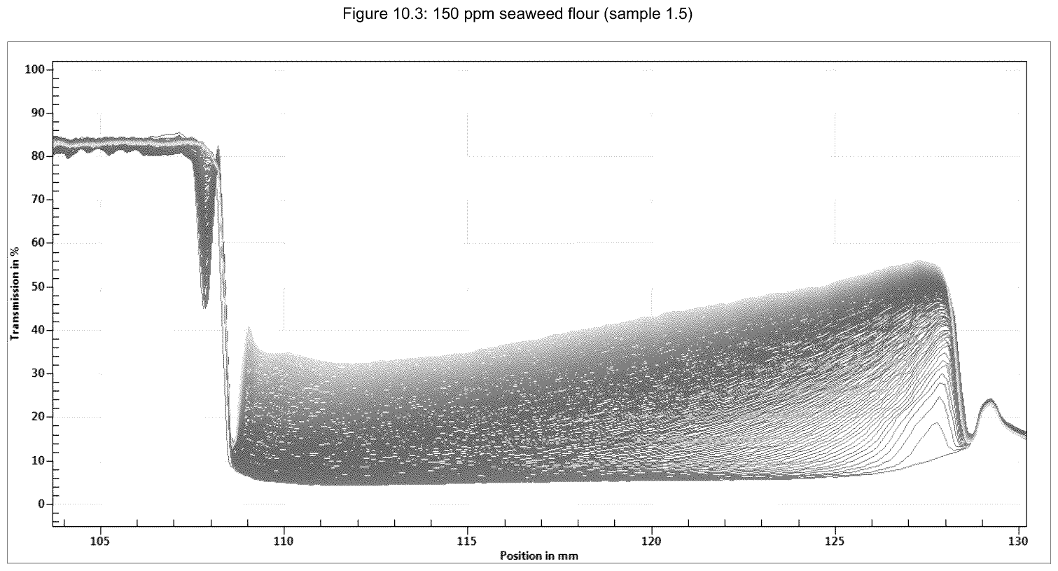

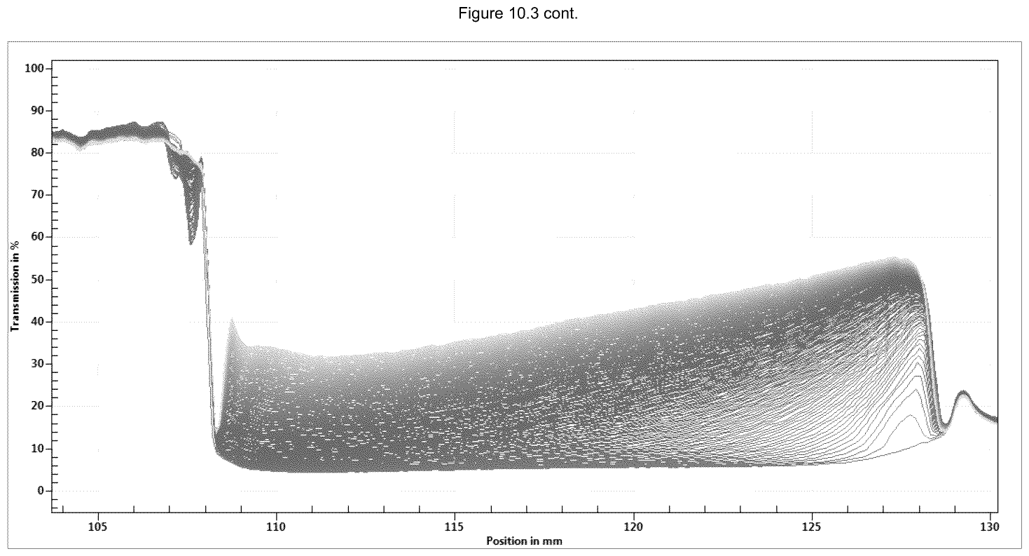

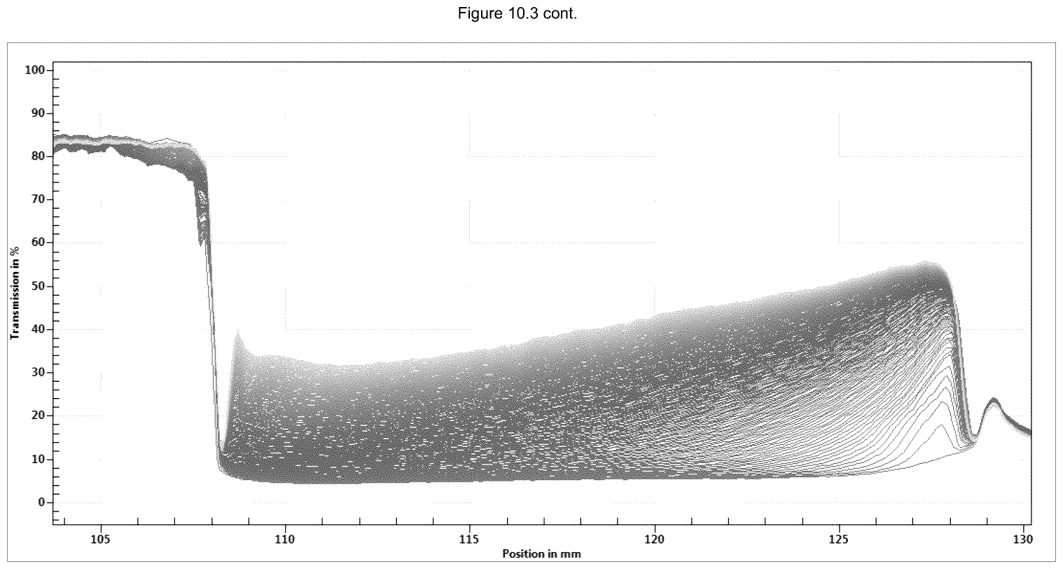

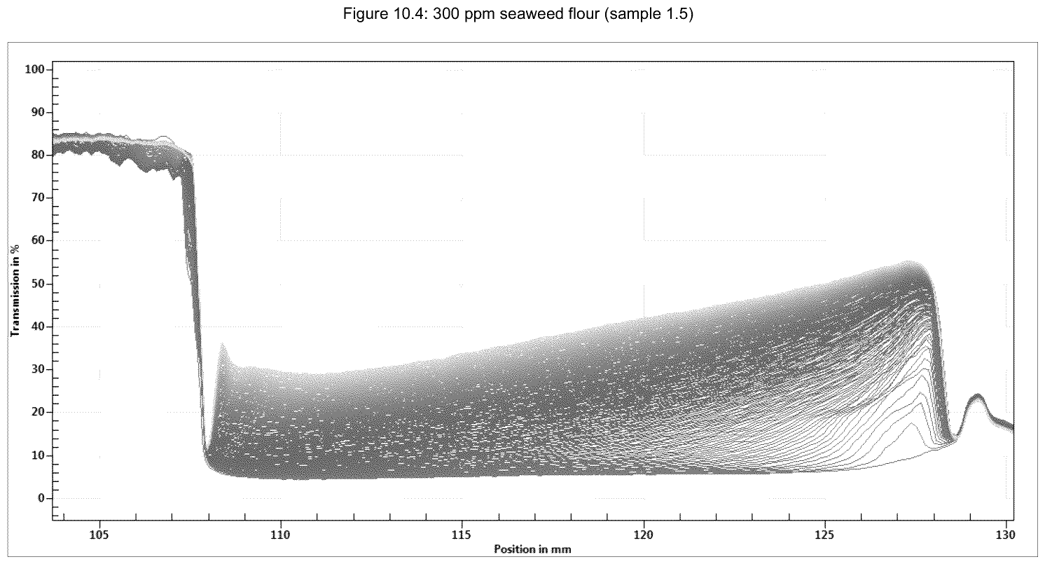

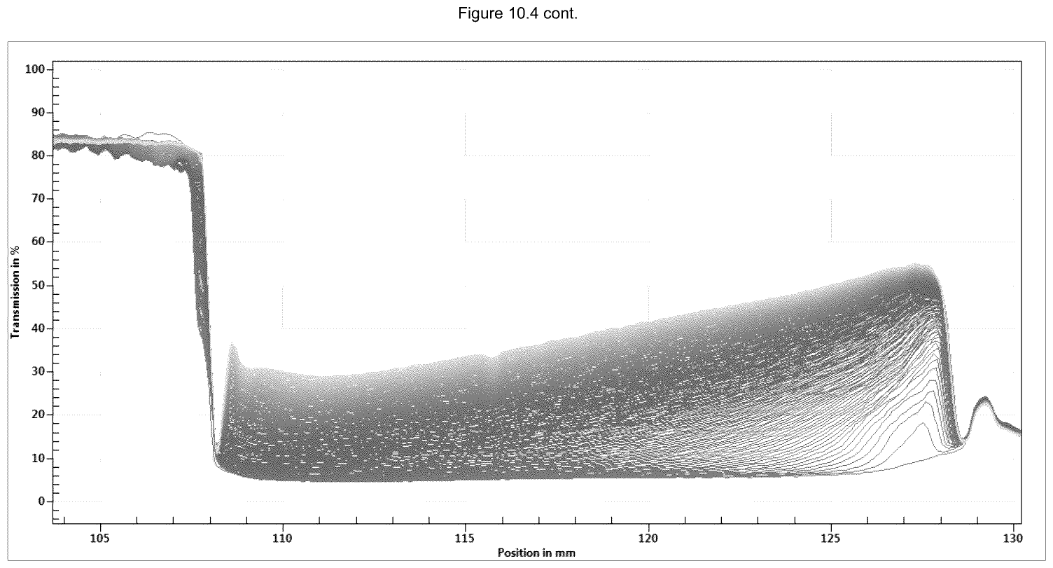

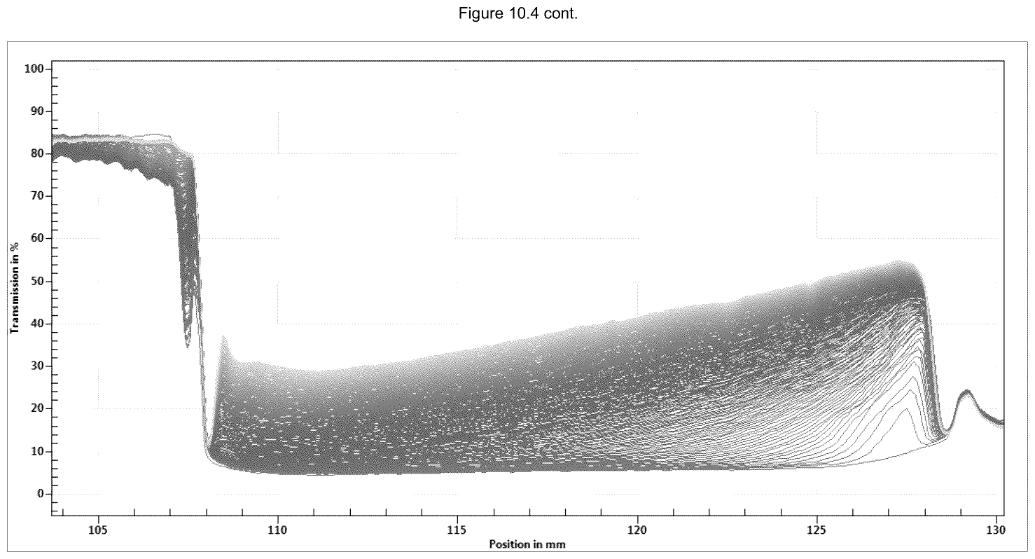

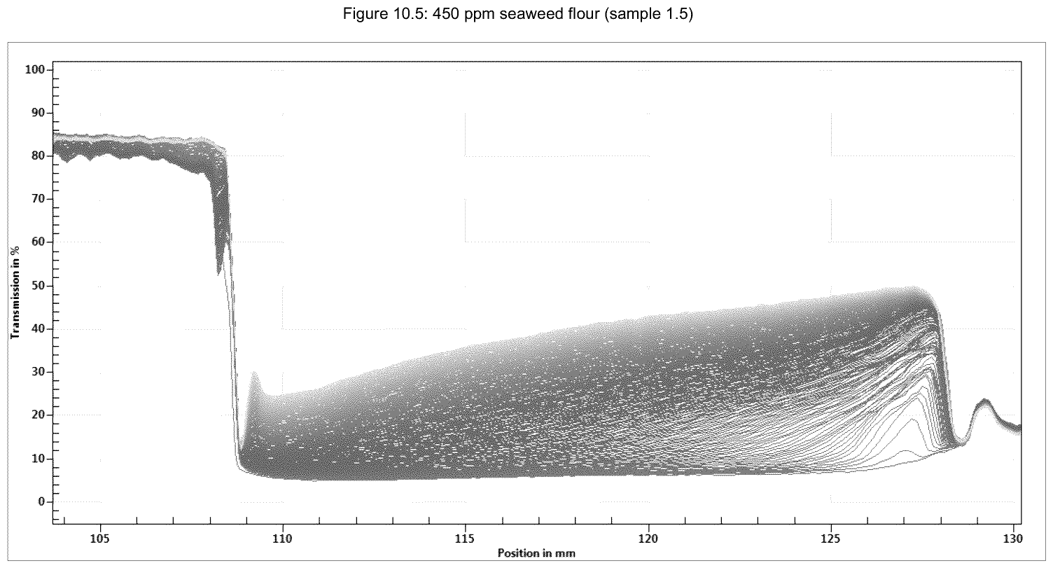

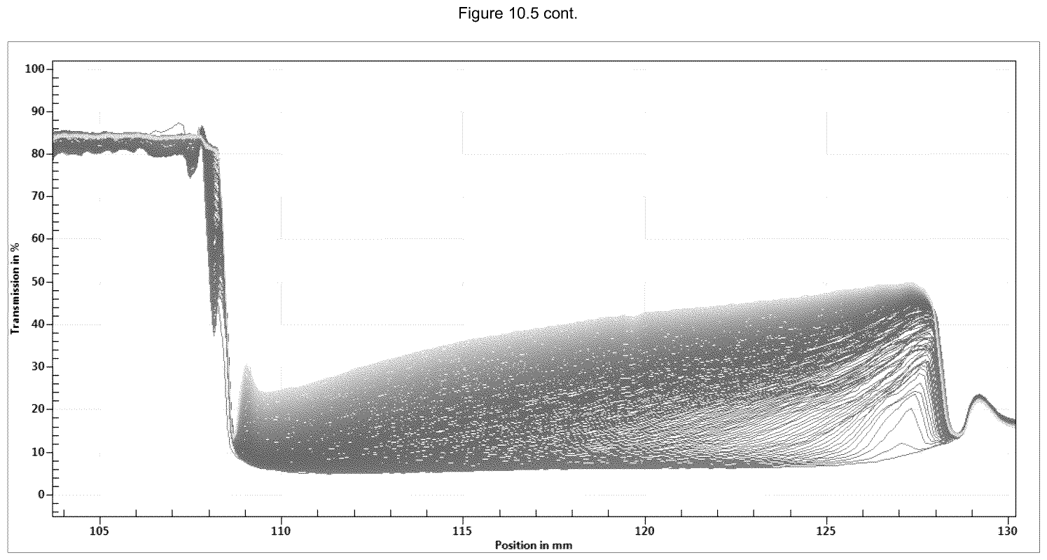

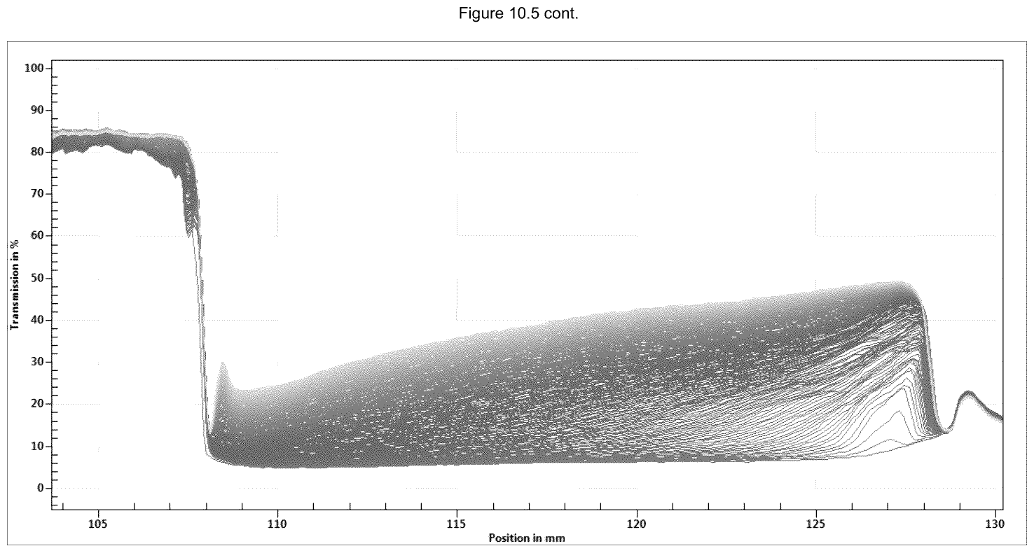

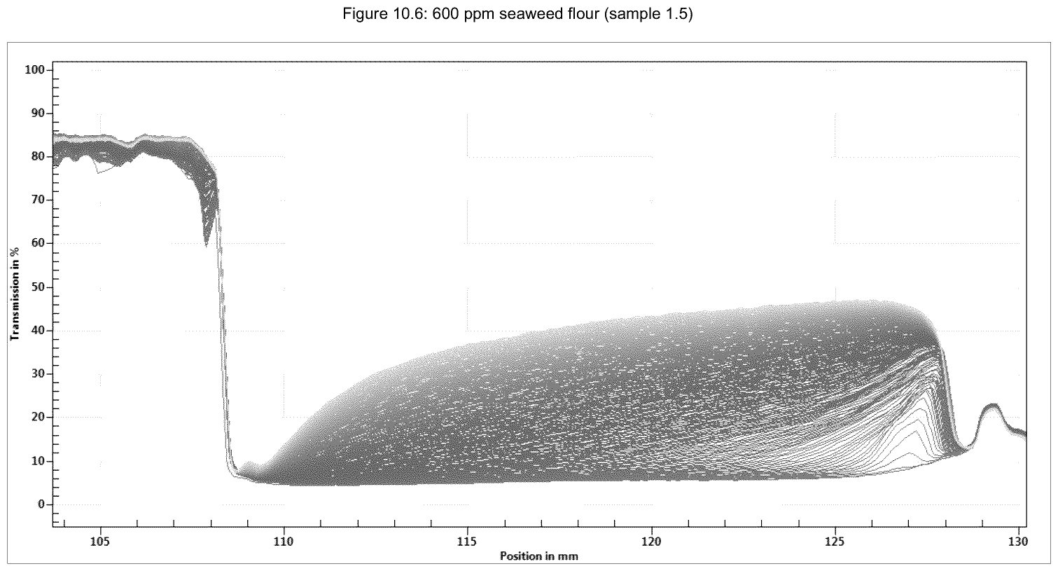

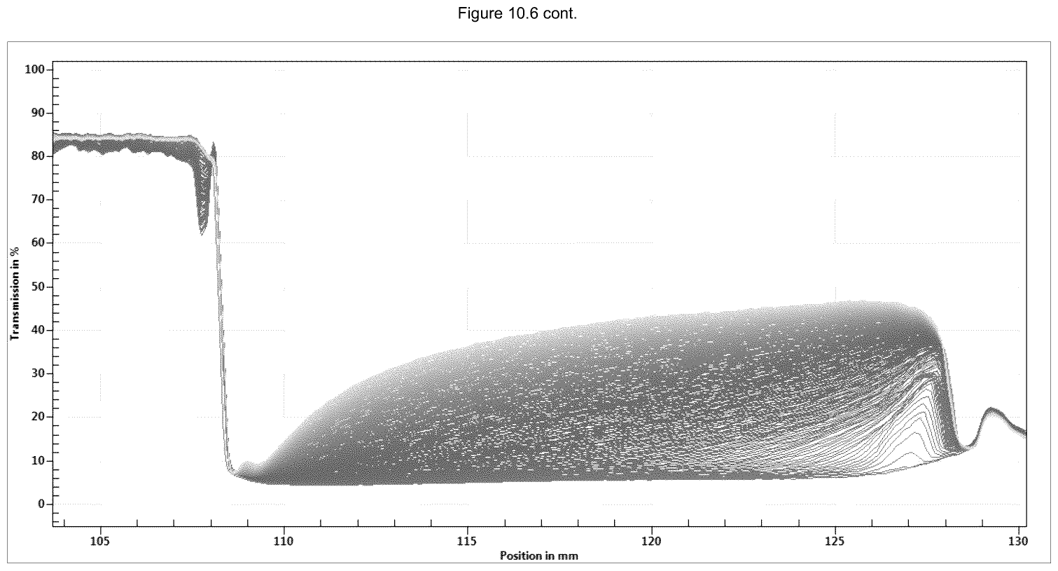

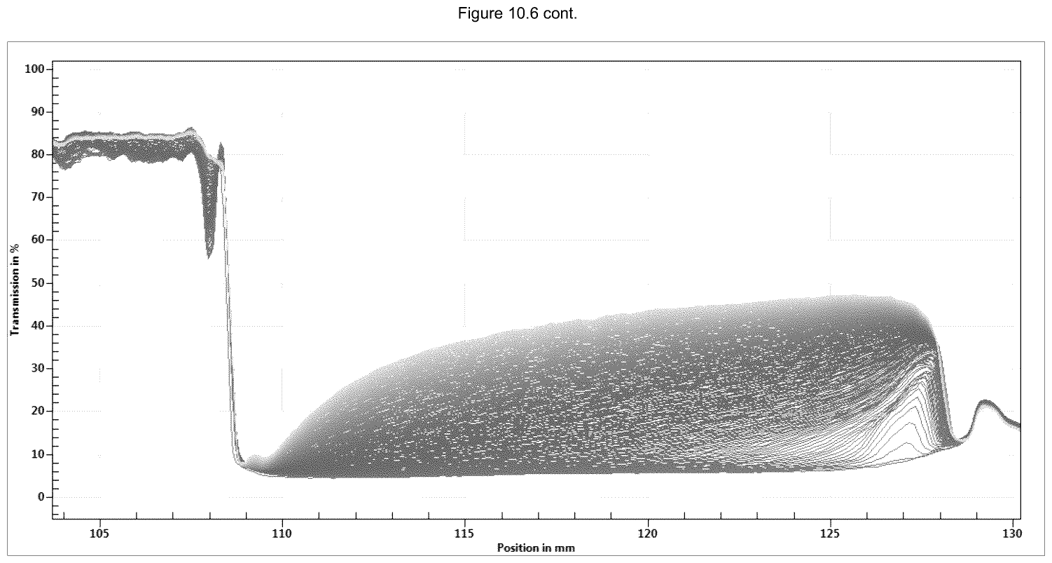

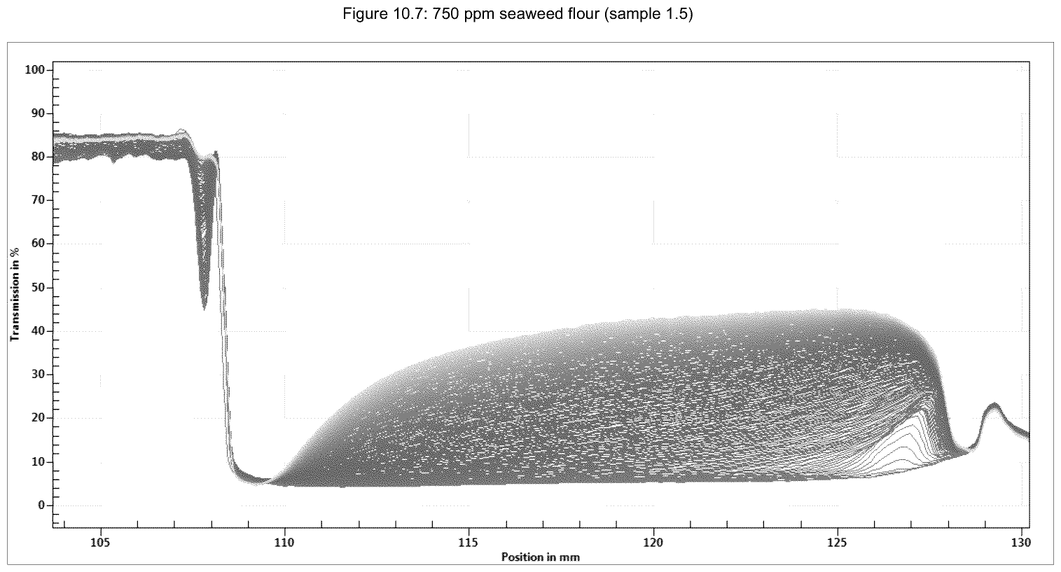

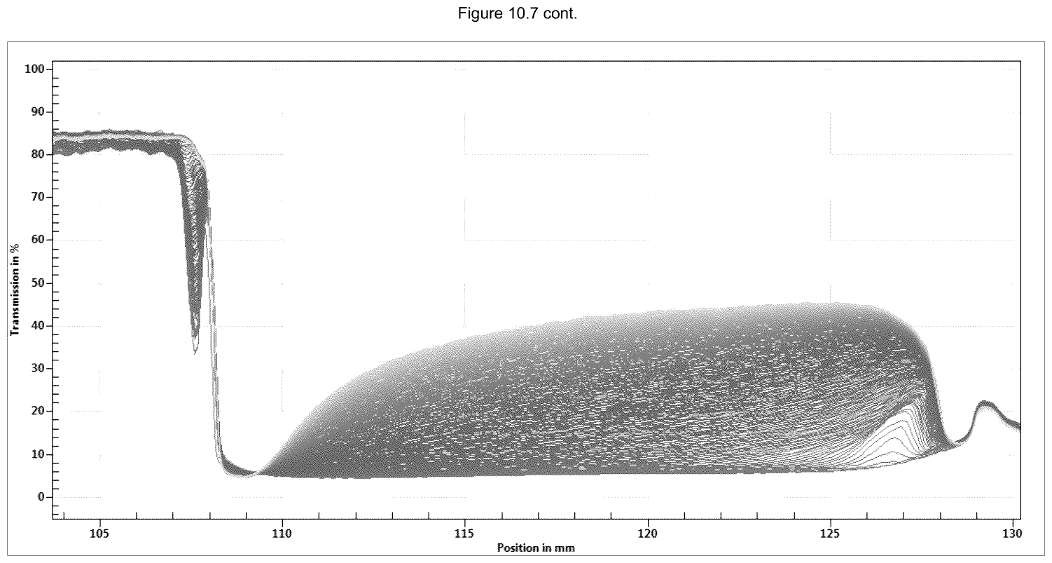

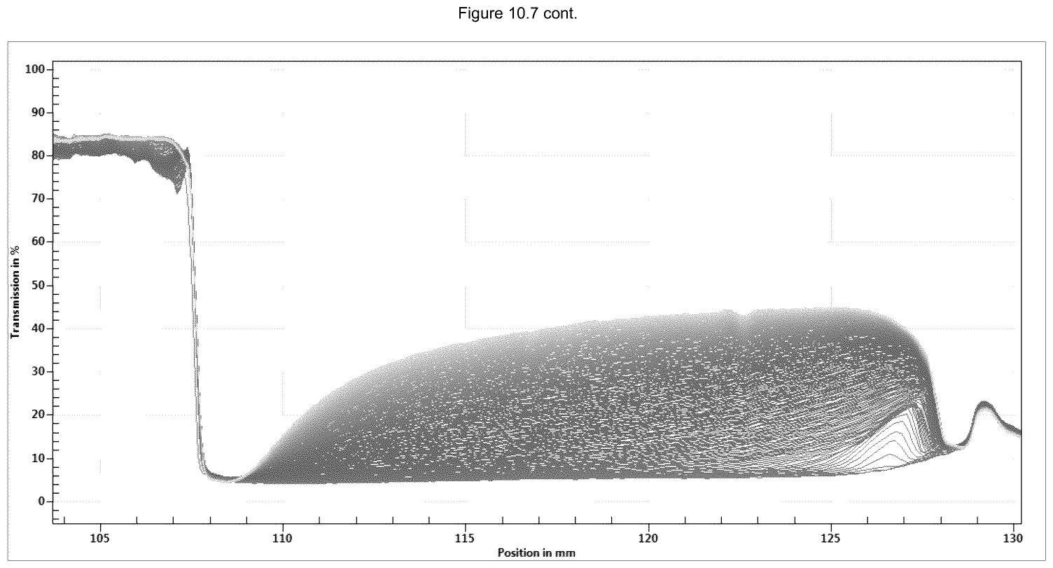

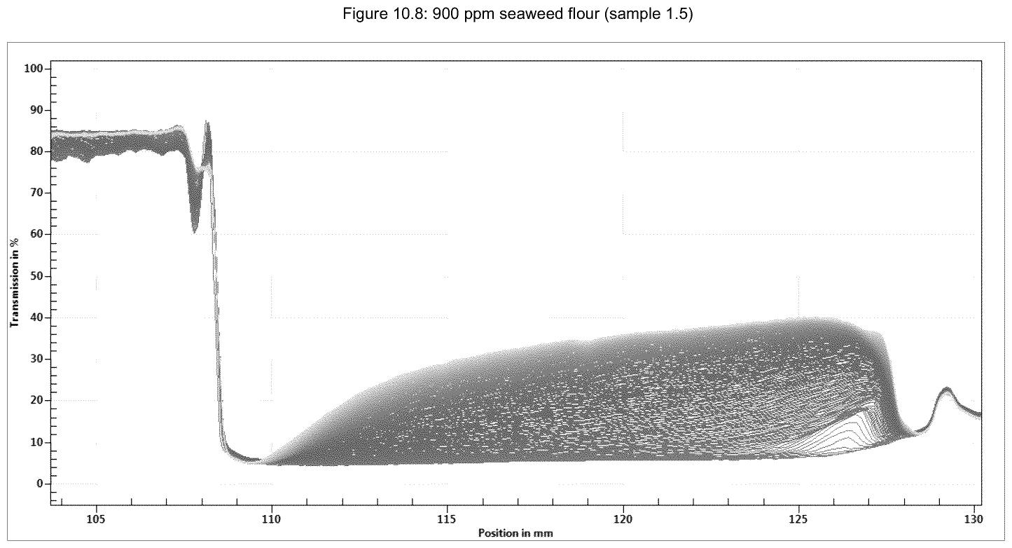

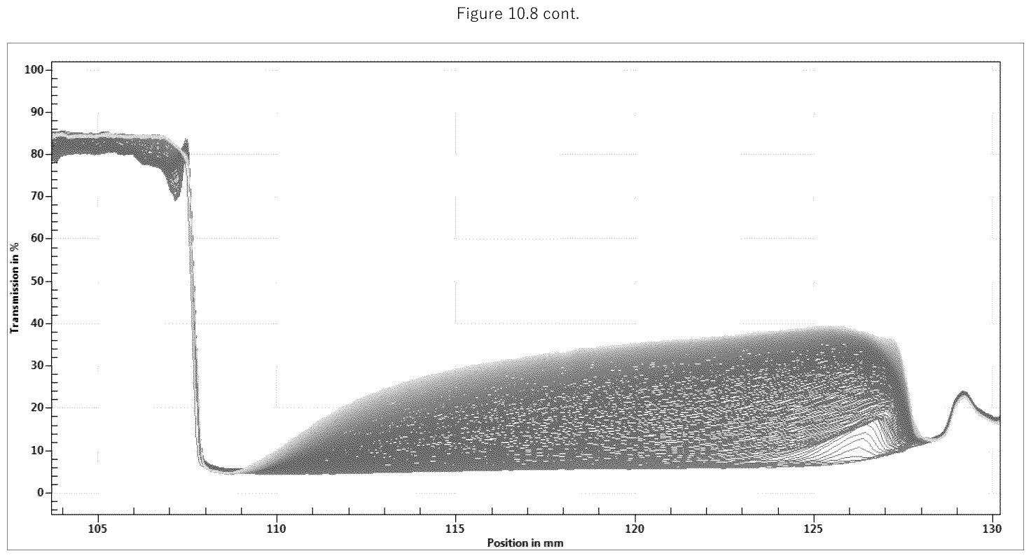

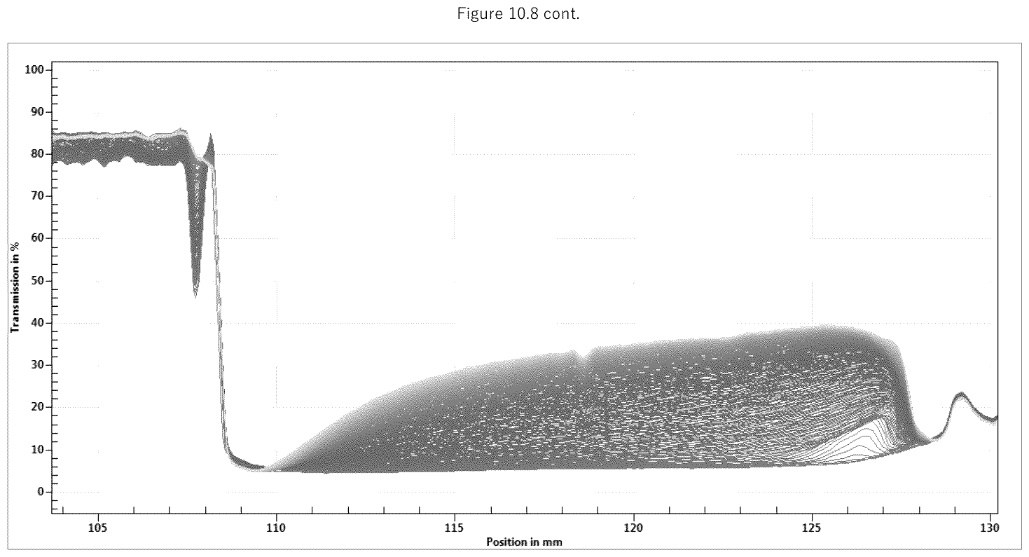

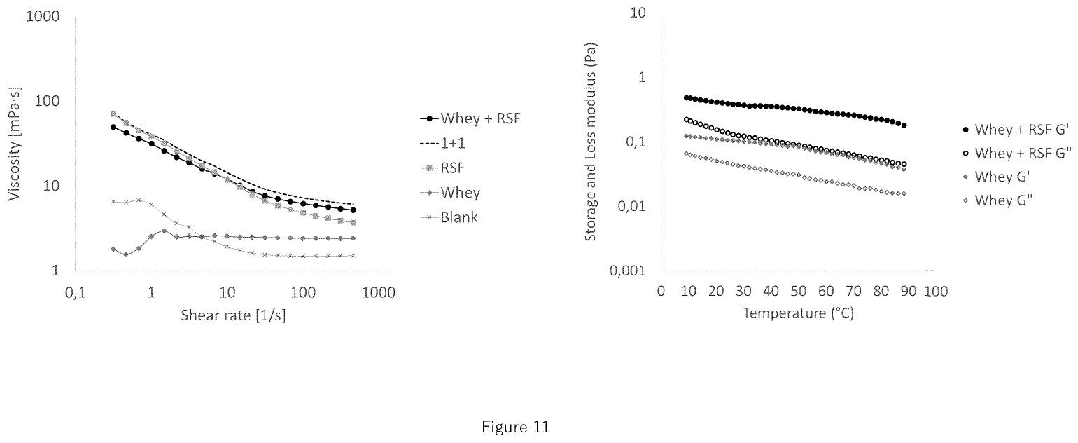

The LUMiSizer plots (FIG. 10) are interpreted in the following way; the bottom of the centrifuge tube is located on the right side of the plot. The steep increase on the left is where the top of the liquid is located. Transparency is plotted on the y-axis and centrifugation time goes from red to green over time. The Lumisizer plots of the seaweed flour (sample 1.5) series show a different curvature arising between the stable samples (600 ppm) and the unstable samples below 600 ppm. Hinting that there is a possible difference in the stabilization mechanism of seaweed flour (sample 1.5) and the refined iota carrageenan control at 200 ppm.

FIG. 9 and FIG. 10 show that soy protein isolate can be stabilized with seaweed flour (sample 1.5). A noticeable change in the LUMiSizer response for seaweed flour (sample 1.5) at 600 ppm and higher occurs at the same dosage levels where visual stabilization is observed after three months of refrigerated storage. Displaying that seaweed flour (sample 1.5) can have a stabilizing effect in plant-based beverages when dosed at the right levels.

### EXAMPLE 6—(Plant-Based) Dessert

### Sample Preparation:

Heat water to 75° C., add proteins to water and hydrate for 30 minutes.
Add dry blended powder ingredients to water (T=60° C.).
Melt fat at 75° C., add fat to water phase under agitation.
For dairy based trials: dry blend powder ingredients and add to milk/cream mixture under good agitation at 4-8° C.
Homogenization at 75 bar (T=58° C.).
Heat treatment with the following parameters:
30 second preheat at 90° C. followed by 15 seconds at 135° C. in a tubular heat exchanger, with subsequent cooling to 15° C.
Filling in 150 ml cups and subsequent storage at 5° C.

### Recipe and Results:

Dairy dessert (Skimmed milk, 2 wt. % Skimmed milk powder):
Seaweed flour (sample P2-2020028) at 0.35 wt. % and 0.6 wt. %
Commercial reference: dessert (0.3 wt. % GRIN DSTED® PECTIN LC 950)
Soy dessert (3.8 wt. % SUPRO® XT 219D IP):
Seaweed flour (sample P2-2020028) at 0.35 wt. % and 0.6 wt. %
Commercial reference: dessert (0.3 wt. % GRIN DSTED® PECTIN LC 950)
Pea dessert (3.5 wt. % TRUPRO™ 2000):
Seaweed flour (sample P2-2020028) at 0.35 wt. % and 0.6 wt. %
Commercial reference: dessert (0.3 wt. % GRINDSTED® PECTIN LC 950)
Viable (plant-based) desserts have been made, demonstrated and reported here.
Strong synergy is unexpectedly observed between seaweed flour and pea protein.
Seaweed flour is surprisingly outperforming the commercial reference in both Pea and Soy recipes.
Firmness increased for all desserts over shelf-life, likely attributed to starch.

TABLE 3
Dessert firmness in grams at week 1 and week 8 in grams
for dairy-, pea- and soy-based desserts.
Dessert (Texture analyser)

Firmness (g)

| | week 1 | week 8 |
|---|---|---|
| Dairy - 0.3 wt. % reference | 31 | 38 |
| Dairy - 0.35 wt. % seaweed flour | 22 | 26 |
| Dairy - 0.6 wt. % seaweed flour | 32 | 39 |
| Pea - 0.3 wt. % reference | 14 | 19 |
| Pea - 0.35 wt. % seaweed flour | 28 | 37 |
| Pea - 0.6 wt. % seaweed flour | 51 | 64 |
| Soy - 0.3 wt. % reference | 10 | 13 |
| Soy - 0.35 wt. % seaweed flour | 15 | 18 |
| Soy - 0.6 wt. % seaweed flour | 23 | 32 |

TABLE 4
Dessert consistency index (Pasn) at week 1 and week 5 in grams
for dairy-, pea- and soy-based desserts.
Dessert - Consistency index

| | Week 1 | Week 5 |
|---|---|---|
| Dairy - 0.3 wt. % reference | 489.4 | 495.1 |
| Dairy - 0.35 wt. % seaweed flour | 367.0 | 346.9 |
| Dairy - 0.6 wt. % seaweed flour | 686.0 | 641.6 |
| Pea - 0.3 wt. % reference | 170.9 | 165.5 |
| Pea - 0.35 wt. % seaweed flour | 329.3 | 335.3 |
| Pea - 0.6 wt. % seaweed flour | 709.3 | 772.5 |
| Soy - 0.3 wt. % reference | 175.9 | 149.6 |
| Soy - 0.35 wt. % seaweed flour | 325.6 | 329.2 |
| Soy - 0.6 wt. % seaweed flour | 444.2 | 454.0 |

### EXAMPLE 7—(Plant-Based) Gelatin-Free Mousse

### Sample Preparation:

Mix all dry ingredients.
Heat fat and emulsifier to 70° C. Heat water to 70° C. and add dry mix, leave for 30 minutes hydration.
Heat to 135° C. for 10-15 sec.
Homogenisation at 75° C./150 bar.
Cool to 20° C. and whip on a Mondo-mixer:
   Main pump: 30 L/h
   Backpressure: 2.1 bar
   Mixing head: 1200 rpm
   Overrun (OR) of 100%
Followed by filling and subsequent storage at 5° C.

### Recipe and Results:

Gelatin-free dairy mousse (6 wt. % Skimmed milk powder):
Seaweed flour (sample P2-2020028) at 0.4 wt. %, 0,7 wt. % and 0.9 wt. %
Commercial reference: gelatin-free mousse (0.45 wt. % CREMODAN® MOUSSE 45)
Gelatin-free soy mousse (2.5 wt. % SUPRO® 670 IP):
Seaweed flour (sample P2-2020028) at 0.7 wt. % and 0.9 wt. %
Gelatin-free pea mousse (2.7 wt. % TRUPRO™ 2000):
Seaweed flour (sample P2-2020028) at 0.4 wt. %, 0,7 wt. % and 0.9 wt. %
Strong synergy observed between seaweed flour and pea protein.
Seaweed flour can directly replace the commercial reference at same dosage in Dairy.

Viable (plant-based) gelatin-free mousses have been made, demonstrated and reported here.

Pea protein synergy will allow for lower seaweed flour dosing than expected.

TABLE 5
Mousse firmness in grams at week 1 and percentage of overrun
during the production of dairy-, pea- and soy-based mousses.

| | Firmness (g) - | |
|---|---|---|
| | week 1 | Overrun % |
| Dairy - 0.45 wt. % reference | 35 | 102 |
| Dairy - 0.4 wt. % seaweed flour | 42 | 100 |
| Dairy - 0.7 wt. % seaweed flour | 48 | 88 |
| Dairy - 0.9 wt. % seaweed flour | 41 | 74 |
| Pea - 0.4 wt. % seaweed flour | 100 | 105 |
| Pea - 0.7 wt. % seaweed flour | 102 | 110 |
| Pea - 0.9 wt. % seaweed flour | 88 | 100 |
| Soy - 0.4 wt. % seaweed flour | 29 | 84 |
| Soy - 0.7 wt. % seaweed flour | 35 | 100 |

### EXAMPLE 8—Protein Systems at Meat Alternative Inclusion Levels

### Sample Preparation:

Samples without Protein:
Mix all dry ingredients.
Add water to thermomixer and heat to 90° C.
Add dry ingredients and mix for 2 minutes.
Fill into cans and store at 5° C.
   Samples with Protein:
Mix texturizer and protein if both are used.
Add water to Stephan cutter and add protein(blend).
Apply vacuum and mix for 1 minute.
Scrape down, apply vacuum, and mix for 1 minute.
Add potassium chloride and mix for 1 minute with vacuum.
Fill into cans.
Cook at 90° C. for 1 hour.
Store at 5° C.

### Recipe and Results:

Without protein
Seaweed flour (sample P3-2020028) at 1 wt. %, 1.125 wt. % and 1.2 wt. %
Commercial reference: (1 wt. % refined kappa carrageenan)
Commercial reference: (1 wt. % semi-refined kappa carrageenan)
11.14 wt. % TRUPRO™ 2000
Seaweed flour (sample P3-2020028) at 0.667 wt. %, 1 wt. %, 1.333 wt. % and 1.667 wt. %
Commercial reference: (1 wt. % refined kappa carrageenan)
Commercial reference: (1 wt. % semi-refined kappa carrageenan)
Blank
10.29 wt. % SUPRO® EX 37 HG IP
Seaweed flour (sample P3-2020028) at 1 wt. %
Commercial reference: (1 wt. % refined kappa carrageenan)
Blank
16.67 wt. % GRINDSTED® VEG PRO XP
Seaweed flour (sample P3-2020028) at 1 wt. %
Commercial reference: (1 wt. % refined kappa carrageenan)
Blank

TABLE 6
Firmness in grams for refined kappa carrageenan, semi-refined kappa
carrageenan and seaweed flour containing protein systems at meat
alternative inclusion levels containing pea, soy or carob protein,
and firmness in grams for a reference system without protein.

| | Firmness (g) |
|---|---|
| Without protein - 1 wt. % refined kappa carrageenan | 589 |
| Without protein - 1 wt. % semi-refined kappa carrageenan | 269 |
| Without protein - 1 wt. % seaweed flour | 48 |
| Without protein - 1.125 wt. % seaweed flour | 63 |
| Without protein - 1.2 wt. % seaweed flour | 83 |
| Pea - no texturant | 26 |
| Pea - 1 wt. % refined kappa carrageenan | 721 |
| Pea - 1 wt. % semi-refined kappa carrageenan | 306 |
| Pea - 0.667 wt. % seaweed flour | 66 |
| Pea - 1 wt. % seaweed flour | 127 |
| Pea - 1.333 wt. % seaweed flour | 211 |
| Pea - 1.667 wt. % seaweed flour | 293 |
| Soy - no texturant | 14 |
| Soy - 1 wt. % refined kappa carrageenan | 542 |
| Soy - 1 wt. % seaweed flour | 220 |
| Carob - no texturant | — |
| Carob - 1 wt. % refined kappa carrageenan | 935 |
| Carob - 1 wt. % seaweed flour | 307 |

TABLE 5
Firmness ratio between seaweed flour and refined kappa carrageenan
for protein systems at meat alternative inclusion levels containing pea,
soy or carob protein, and for a reference system without protein.
Firmness ratio - seaweed flour:refined kappa carrageenan

| | (1 wt. % seaweed flour/ |
|---|---|
| | 1 wt. % refined kappa carrageenan) * 100 |
| Without protein | 8.15% |
| Pea | 17.61% |
| Soy | 40.59% |
| Carob | 32.83% |

Seaweed flour benefits more from protein addition compared to refined kappa carrageenan, indicating that seaweed flour, surprisingly, has more potential to interact with protein. Wherein soy protein improves seaweed flour the most in this application, followed by carob protein and pea protein.

### EXAMPLE 9—Plant-Based Emulsion

### Sample Preparation:

Mix dry ingredients except salt.
Add ice water to bowl chopper, add dry ingredients and hydrate with knives running at 1000 rpm and bowl at 25 rpm.
Chop 1 min with knives at 5400 rpm and bowl at 25 rpm.
Add oil and chop 1 minute with knives at 5400 rpm and bowl at 25 rpm.
Add salt and chop 30 seconds with knives at 5400 rpm and bowl at 25 rpm.
Apply vacuum and chop for 1% minutes with knives at 5400 rpm and bowl at 25 rpm.
Fill into cans and cook at 98° C. full steam for 1 hour.
Cool down using cold water shower and store at 5° C.

### Recipe and Results:

9 wt. % SUPRO® EX 37 HG IP
Seaweed flour (sample P3-2020028) at 3.87 wt. %, 5.81 wt. %, 7.74 wt. % and 9.68 wt. %
Commercial reference: (3.82 wt. % refined kappa carrageenan)
Blank
9.75 wt. % TRUPRO™ 2000
Seaweed flour (sample P3-2020028) at 3.87 wt. % and 7.74 wt. %
Commercial reference: (3.82 wt. % refined kappa carrageenan)
Blank

Note that the plant-based emulsion discussed in this example is a component of what will be a viable food-matrix, although it is not regarded as a tenable food matrix in its own right. The seaweed flour protein synergies displayed in this example will however contribute to a viable product, demonstrated in example 10.

TABLE 6
Firmness in grams for refined kappa carrageenan and seaweed flour
containing plant-based emulsions with pea or soy protein, and firmness
in grams for a reference soy-based emulsion without texturant.

| | Firmness (g) |
|---|---|
| Soy - no texturant | 223 |
| Soy - 3.87 wt. % refined kappa carrageenan | 667 |
| Soy - 3.87 wt. % seaweed flour | 800 |
| Soy - 5.81 wt. % seaweed flour | 1062 |
| Soy - 7.74 wt. % seaweed flour | 1230 |
| Soy - 9.68 wt. % seaweed flour | 1615 |
| Pea - 3.87 wt. % refined kappa carrageenan | 726 |
| Pea - 3.87 wt. % seaweed flour | 765 |
| Pea - 7.74 wt. % seaweed flour | 1251 |

TABLE 7
Firmness ratio between seaweed flour and refined kappa carrageenan
for plant-based emulsions containing soy or pea protein.
Firmness ratio - seaweed flour:refined kappa carrageenan

| | (3.87 wt. % seaweed flour/ |
|---|---|
| | 3.87 wt. % refined kappa carrageenan) * 100 |
| Soy-based | 119.94% |
| Pea-based | 105.37% |

Seaweed flour clearly outperforms refined kappa carrageenan at the same dosage level. Indicating that this texturant to protein ratio is even more beneficial to the performance of seaweed flour compared to refined kappa carrageenan. This can be taken as further verification of seaweed flour's unique synergy with plant-based proteins.

### EXAMPLE 10—Plant-Based Cold-Cut

### Sample Preparation:

Hydrate textured proteins under vacuum and grind textured proteins at 25 mm scale.
Mix dry ingredients except salt.
Add ice water to bowl chopper, add dry ingredients and hydrate with knives running at 1000 rpm and bowl at 25 rpm.
Chop 1 min with knives at 5400 rpm and bowl at 25 rpm.
Add oil and chop 1 minute with knives at 5400 rpm and bowl at 25 rpm.
Add salt and chop 30 seconds with knives at 5400 rpm and bowl at 25 rpm.
Apply vacuum and chop for 1% minutes with knives at 5400 rpm and bowl at 25 rpm.
Add grinded textured proteins and mix for 1 minute with knives at 500 rpm and bowl at 25 rpm.
Apply vacuum and mix for 1 minute with knives at 500 rpm and bowl at 25 rpm.
Fill into 60mm casing.
Fill into cans and cook at 98° C. full steam for 1 hour.
Cool down using cold water shower and store at 5° C.

### Recipe and Results:

Plant-based cold-cut (2.52 wt. % SUPRO® EX 37 HG IP, 20.02 wt. % SUPRO® MAX 5050 IP)
Seaweed flour (sample P3-2020028) at 1.08 wt. %, 1.63 wt. %, 2.17 wt. % and 2.71 wt. %
Commercial reference: (1.08 wt. % refined kappa carrageenan)
Plant-based cold-cut (2.52 wt. % SUPRO® EX 37 HG IP, 20.02 wt. % SUPRO® MAX 5050 IP)
Seaweed flour (sample P3-2020028) at 1.08 wt. % and 2.17 wt. %
Commercial reference: (1.08 wt. % refined kappa carrageenan)

TABLE 8
Firmness in grams for refined kappa carrageenan and seaweed flour
containing plant-based cold-cuts with soy or pea protein, and firmness
in grams for a reference soy-based cold-cut without texturant.

| | Firmness (g) |
|---|---|
| Soy - no texturant | 1429 |
| Soy - 1.08 wt. % refined kappa carrageenan | 3525 |
| Soy - 1.08 wt. % seaweed flour | 2399 |
| Soy - 1.63 wt. % seaweed flour | 2874 |
| Soy - 2.17 wt. % seaweed flour | 3463 |
| Soy - 2.71 wt. % seaweed flour | 3853 |
| Pea - 1.08 wt. % refined kappa carrageenan | 3585 |
| Pea - 1.08 wt. % seaweed flour | 2475 |
| Pea - 2.17 wt. % seaweed flour | 3497 |

TABLE 9
Firmness ratio between seaweed flour and refined kappa carrageenan
for plant-based cold-cuts containing soy or pea protein.
Firmness ratio - seaweed flour:refined kappa carrageenan

| | (1.08 wt. % seaweed flour/ |
|---|---|
| | 1.08 wt. % refined kappa carrageenan) * 100 |
| Soy-based cold-cut | 68.06% |
| Pea-based cold-cut | 69.04% |

### EXAMPLE 11—Plant-Based Sausage (Frankfurter-Style)

### Sample Preparation:

Add ice water and cold oil are added to the cold bowl chopper.
Mix all dry ingredients, except spices and salt and add to the bowl and chopped at slow speed. Knives running at 1000 rpm and bowl at 25 rpm, until all dry ingredients are hydrated.
Finally, they are chopped for 1 minute with knives at 5400rpm and bowl at 25 rpm.
Add spices and salt and chop for 2 minutes with knives at 5400 rpm and bowl at 25 rpm.
Fill the emulsion into 22 mm casings.
Smoke at 55° C. for 20 minutes and then steam cook at 92° C. until the core temperature is 88° C. Hold core temperature of 88° C. for 5 minutes.
Cool down using cold water shower and store at 5° C.

### Recipe and Results:

Plant-based sausage (10.3 wt. % SUPRO® EX 37 HG IP)
Seaweed flour (50/50 blend of sample 38.1 and 38.2) at 1.6 wt. %, 2.0 wt. % and 2.5 wt. %
Commercial reference: (1.6 wt. % refined kappa carrageenan)

TABLE 10
Break force at 5° C. and 70° C. in grams for
soy-based plant-based sausages containing refined kappa
carrageenan or seaweed flour at different concentrations.

Break force (g)

| | Cold texture | Warm texture |
|---|---|---|
| | (5° C.) | (70° C.) |
| Soy - 1.4 wt. % refined kappa | 1607 | 2452 |
| carrageenan | | |
| Soy - 1.4 wt. % seaweed flour | 1083 | 2818 |
| Soy - 2.0 wt. % seaweed flour | 1058 | 2905 |
| Soy - 2.5 wt. % seaweed flour | 1175 | 2714 |

TABLE 11
Break Force ratio between seaweed flour and refined kappa
carrageenan for plant-based sausages containing soy protein.
Break force ratio - seaweed flour:refined kappa carrageenan

| | (1.08 wt. % seaweed flour/ |
|---|---|
| | 1.08 wt. % refined kappa carrageenan) * 100 |
| Soy-based sausage | 67.39% |

### EXAMPLE 12 (Plant-Based) Frozen Dessert

Sample preparation:
Mix dry ingredients.
Mix dry, liquid ingredients and fat. Increase temperature to 70° C.
Homogenize at: 78° C./160 bar.
Pasteurize at: 84° C./30 sec.
Cool to 5° C.
Ageing overnight in ice water.
Freezing, light extrusion with 100% overrun, drawing temperature: -5.5° C.
Fill in 250 ml rectangular paper containers.
Overnight freezing in hardening tunnel at -30° C.
Store at −25° C.
The rheology of the samples was evaluated at 5° C.
Samples were tempered at −18° C. before sensorial evaluation.

### Recipe and Results:

Frozen dairy dessert (8 wt. % Skimmed milk powder, 2.5 wt. % Whey powder)
Seaweed flour (sample P2-2020028) at 0.04 wt. %
Commercial reference: frozen dairy dessert (0.02 wt. % refined kappa carrageenan)
Blank
Frozen soy dessert (2.5 wt. % SUPRO® XT 221D IP)
Seaweed flour (sample P2-2020028) at 0.04 wt. %
Commercial reference: frozen soy dessert (0.02 wt. % refined kappa carrageenan)
Blank
At 0.04 wt. % seaweed flour can be used to replace 0.02 wt. % refined kappa carrageenan for stabilization of the ice cream mix during ageing time.

Further aspects of the invention are described below in the following numbered paragraphs.
1. A food ingredient obtained from seaweed of the class of Rhodophyta wherein
   (a) the food ingredient contains mu carrageenan in an amount of at least 4 wt. % based on the total weight of the food ingredient; and
   (b) the weight average molecular weight of carrageenan present in the food ingredient is at least 700 kDa, such as at least about 710, 720, 730, 740, 750, 760, 770, 780, or 790 kDa or at least 800 kDa.
2. A food ingredient according to paragraph 1 wherein the seaweed is at least one seaweed of the genera Kappaphycus, Eucheuma, Gigartina, Chondrus, lriadae, Mazzaella, Mastocarpus, Sarcothalia, Hypnea, Furcellaria, Gracilaria, Gelidium, Gelidiella. Pterocladia, Halymenia and Chondracanthus.
3. A food ingredient according to paragraph 1 or 2 wherein the seaweed is at least of the genus Eucheuma or Kappaphycus.
4. A food ingredient according to any one of paragraphs 1 to 3 wherein the seaweed is at least of the species Euchema striatum/Kappaphycus striatus, Euchema alvarezii/Kappaphycus alvarezii, Eucheuma cottonii/Kappaphycus cottonii, or a combination thereof, such as the combination of Eucheuma cottonii and Kappaphycus alvarezii.
5. A food ingredient according to any one of paragraphs 1 to 4 wherein the seaweed is at least of the species Kappaphycus striatus and Kappaphycus alvarezii.
6. A food ingredient according to any one of paragraphs 1 to 5 wherein the food ingredient contains mu carrageenan in an amount of at least 5 wt. % based on the total weight of the food ingredient.
25 7. A food ingredient according to any one of paragraphs 1 to 6 wherein the food ingredient contains mu carrageenan in an amount of at least 6 wt. % based on the total weight of the food ingredient.
8. A food ingredient according to any one of paragraphs 1 to 7 wherein the food ingredient contains mu carrageenan in an amount of from 6 to 20 wt. % based on the total weight of the food ingredient.
9. A food ingredient according to any one of paragraphs 1 to 8 wherein the weight average molecular weight of carrageenan present in the food ingredient is at least 700 KDa, or at least 800, 900, 1000, 1100, or 1200 kDa
10. A food ingredient according to any one of paragraphs 1 to 9 wherein the food ingredient contains acid insoluble matter in an amount of from 3 to 20 wt. % based on the total weight of the food ingredient.
11. A food ingredient according to any one of paragraphs 1 to 10 wherein the food ingredient contains acid insoluble matter in an amount of from 5 to 15 wt. % based on the total weight of the food ingredient.
12. A food ingredient according to any one of paragraphs 1 to 11 wherein the food ingredient contains acid insoluble matter in an amount of from 8 to 15 wt. % based on the total weight of the food ingredient.
13. A food ingredient according to any one of paragraphs 1 to 12 wherein the food ingredient contains acid insoluble matter in an amount of from 8.5 to 10.5 wt. % based on the total weight of the food ingredient.
14. A food ingredient according to any one of paragraphs 1 to 13 wherein the food ingredient contains kappa carrageenan in an amount of no greater than 70 wt. %, such as no greater than 65 wt. %, such as no greater than 60 wt. %, such as no greater than 50 wt. % based on the total weight of the food ingredient.
15. A food ingredient according to any one of paragraphs 1 to 14 wherein the food ingredient contains kappa carrageenan in an amount of no greater than 55 wt. % based on the total weight of the food ingredient.
16. A food ingredient according to any one of paragraphs 1 to 15 wherein the food ingredient contains kappa carrageenan in an amount of from 20 to 70 wt. %, such as from 20 to 65 wt. %, such as from 40 to 60 wt. %, such as from 50 to 60 wt. %, or from 30 to 55 wt. % based on the total weight of the food ingredient.
17. A composition comprising
   (i) a food ingredient as defined in any one of paragraphs 1 to 16; and
   (ii) a protein.

18. A composition according to paragraph 17 wherein the protein is a protein concentrate or isolate.
19. A composition according to paragraph 17 or 18 wherein the protein is selected from legumenous proteins, such as soy, pea, faba, carob; cereal proteins, such as oat, rice, wheat, oat; algal protein, grass protein, cotton protein, dairy proteins, such as whey, casein; and any mixture thereof.
20. A composition according to paragraph 17, 18 or 19 wherein the protein is selected from soy protein, pea protein, and mixtures thereof.
21. A composition according to paragraphs 17 to 20, wherein the protein is in an amount of 10 0.0-20.0 wt. %, such as in the amount of 3.0-20.0 wt. %, such as in the amount of 3.0-18.0 wt.

%, such as in the amount of 3.0-16.0 wt. %, such as in the amount of 3.0-14.0 wt. %, such as in the amount of 3.0-12.0 wt. %, such as in the amount of 3.0-10.0 wt. %, such as in the amount of 3.0-9.0 wt. %, such as in the amount of 3.0-8.0 wt. %, such as in the amount of 3.0-4.5 wt. %, such as in the amount of 3.5-4.5 wt. %, such as in the amount of 3.5-4 wt. %.
22. A composition comprising
   (i) a food ingredient as defined in any one of paragraphs 1 to 16; and
   (ii) pea protein.
23. A composition comprising
   (i) a food ingredient as defined in any one of paragraphs 1 to 16; and
   (ii) a polysaccharide selected from galactomannans, glucomannans, and mixtures thereof.
24. A composition comprising
   (i) a food ingredient as defined in any one of paragraphs 1 to 16; and
   (ii) a hydrocolloid/binder, such as methylcellulose.
25. A composition comprising
   (i) a food ingredient as defined in any one of paragraphs 1 to 16; and
   (ii) a fiber, such as carob fiber.
26. A composition comprising
   (i) a food ingredient as defined in any one of paragraphs 1 to 16; and
   (ii) a protein; and
   (iii) a polysaccharide selected from galactomannans, glucomannans, and mixtures thereof, and
   (iv) optionally a salt, such as potassium chloride.
27. A foodstuff containing a food ingredient as defined in any one of paragraphs 1 to 16 or a composition as defined in any one of paragraphs 17 to 26.
28. A foodstuff according to paragraph 27 wherein the food ingredient is present in an amount of no greater than 7 wt. % based on the total weight of the foodstuff.
29. A foodstuff according to paragraph 27 wherein the food ingredient is present in an amount of 0.1 to 5 wt. %, such as 0.3 to 1 wt. %, such as 0.3 to 0.7 wt. %, such as 0.3 to 0.6 wt. %, such as 0.3 to 0.5 wt. %, such as 0.3 to 0.45 wt. %, such as 0.3 to 0.4 wt. %, or 0.35 to 0.45 wt. %, based on the total weight of the foodstuff.
30. A foodstuff according to any one of paragraphs 27 to 29 wherein when the foodstuff is a dairy foodstuff, such as a mousse or a creamer, or dessert, such as a dairy foodstuff wherein the food ingredient is present in an amount of less than 8.4 wt. %, such as less than 6 wt. %, such as less than 4 wt. %, or less than 2 wt. %, or less than 1 wt. %, or less than 0.5 wt. %, or in the range of 0.02% wt. to 1 wt. %, such as in the range of 0.1% wt. to 1 wt. %, or is present in an amount of greater than 9.3 wt. %, based on the total weight of the foodstuff.
31. A foodstuff according to any one of paragraphs 27 to 30 wherein the food ingredient is present in an amount of less than 8.4 wt. %, such as less than 6 wt. %, such as less than 4 wt. %, or less than 2 wt. %, or less than 1 wt. %, or less than 0.5 wt. %, or in the range of 0.02% wt. to 1 wt. % or is present in an amount of greater than 9.3 wt. %, based on the total weight of the foodstuff.
32. A foodstuff according to any one of paragraphs 27 to 29, and 31 wherein the foodstuff is a spread, a plant-based emulsion, or a meat alternative, such as a sausage, a cold cut, or a burger.
33. A foodstuff according to any one of paragraphs 27 to 29, and 31 wherein the foodstuff is a dessert, a mousse, a creamer, or a frozen dessert, such as an ice-cream.
34. A foodstuff according to paragraphs 33 wherein the foodstuff is a dessert with a firmness in grams at week 1 higher than about 12 as measured according to the assay used for example 6.
35. A foodstuff according to paragraphs 33 wherein the foodstuff is a mousse with a firmness in grams at week 1 higher than about 20, such as higher than about 30, 40, 50, 60, or as measured according to the assay used for example 7.
36. A foodstuff according to paragraphs 32 wherein the foodstuff is a meat alternative with a firmness in grams higher than about 60, such as higher than about 70, 80, 90, 100, 120, 140, 160, 180, or 200 as measured according to the assay used for example 8.
37. A foodstuff according to paragraphs 32 wherein the foodstuff is a plant-based emulsion with a firmness in grams higher than about 700, such as higher than about 750, 800, 850, 900, 950, 1000, 1100, 1200, 1300, 1400, or 1500 as measured according to the assay used for example 9.
38. A foodstuff according to paragraphs 32 wherein the foodstuff is a plant-based cold-cut with a firmness in grams higher than about 2000, such as higher than about 2200, 2400, 2600, 2800, 3000, 3200, 3400, or 3600 as measured according to the assay used for example 10.
39. A foodstuff according to paragraphs 32 wherein the foodstuff is a plant-based sausage with a firmness in grams higher than about 2500, such as higher than about 2600, 2700, 2800, or 2900 as measured at 70° C. according to the assay used for example 11.
40. The foodstuff according to any one of paragraphs 32 to 39, comprising a protein selected from legumenous proteins, such as soy, pea, faba, carob; cereal proteins, such as oat, rice, wheat, oat; algal protein, grass protein, cotton protein, dairy proteins, such as whey or casein; and any mixture thereof.
41. The foodstuff according to paragraph 40, comprising a protein selected from soy and/or pea.
42. The foodstuff according to paragraphs 40 or 41, wherein the foodstuff is a dessert, a mousse, or a creamer, such as an ice-cream and wherein the protein is in an amount of 0.2-wt %, such as in the amount of 0.2-8.0 wt %, such as in the amount of 0.2-6 wt %, such as in the amount of 0.2-4.0 wt %, or in the amount of 0.5-8.0 wt %, such as in the amount of 1.0-8.0 wt %, such as in the amount of 2.0-8.0 wt %, such as in the amount of 3.0-8.0 wt %, such as in the amount of 4.0-8.0 wt %, such as in the amount of 3.0-4.5 wt %, such as 3.0-5.0 wt %, such as in the amount of 3.0-4.5 wt %, such as in the amount of 3.5-4.5 wt %, such as in the amount of 3.5-4.0 wt %.
43. The foodstuff according to paragraphs 40 or 41, wherein the foodstuff is a spread, or a meat alternative, such as a sausage, a cold cut, or a burger and wherein the protein is in an amount of 0.0-20.0 wt %, such as in the amount of 1.0-20.0 wt %, such as in the amount of 2.0-20.0 wt %, such as in the amount of 3.0-20.0 wt %, such as in the amount of 4.0-20.0 wt %, such as in the amount of 5.0-20.0 wt %, such as in the amount of 6.0-20.0 wt %, such as in the amount of 7.0-20.0 wt %, such as in the amount of 8.0-20.0 wt %, such as in the amount of 8.0-19.0 wt %, such as in the amount of 8.0-18.0 wt %, such as in the amount of 8.0-17.0 wt %, such as in the amount of 7.0-17.0 wt %, such as in the amount of 7.0-15.0 wt %, such as in the amount of 7.0-13.0 wt %, such as in the amount of 7.0-11.0 wt %.
44. A foodstuff according to any one of paragraphs 27 to 29 wherein when the foodstuff is a beverage, such as a beverage wherein the protein is in an amount of 0.2-10.0 wt %, such as in the amount of 0.2-8.0 wt %, such as in the amount of 0.2-6 wt %, such as in the amount of 0.2-4.0 wt %, or in the amount of 0.5-8.0 wt %, such as in the amount of 1.0-8.0 wt %, such as in the amount of 2.0-8.0 wt %, such as in the amount of 3.0-8.0 wt %, such as in the amount of 4.0-8.0 wt %, such as in the amount of 3.0-5.0 wt %, such as in the amount of 3.0-4.5 wt %, such as in the amount of 3.5-4.5 wt %, such as in the amount of 3.5-4.0 wt %.
45. A method for improving the stability of a foodstuff, the method comprising the step of combining with the foodstuff a food ingredient as defined in any one of paragraphs 1 to 16 or a composition as defined in any one of paragraphs 17 to 26.
46. Use of a food ingredient as defined in any one of paragraphs 1 to 16 or a composition as defined in any one of paragraphs 17 to 26 for improving the stability of a foodstuff.
47. A method for reducing sedimentation in a beverage, the method comprising the step of combining with the beverage a food ingredient as defined in any one of paragraphs 1 to 16 or a composition as defined in any one of paragraphs 17 to 26.
48. Use of a food ingredient as defined in any one of paragraphs 1 to 16 or a composition as defined in any one of paragraphs 17 to 26 for reducing sedimentation in a beverage.
49. A process for preparing a food ingredient as defined in any one of paragraphs 1 to 16 the process comprising the steps of:
   (a) providing seaweed of the class of Rhodophyta,
   (b) drying the seaweed of step (a), and optionally reducing color by natural sunlight;
   (c) rehydrating the dried seaweed at a temperature of from 20° C. to 85° C., such as from 50° C. to or from 60° C. to 70° C., normally in the presence of a salt solution, such as NaCl and/or KCl and/or CaCl₂ at a pH that is lower than 9.5, such as lower than 8.5, such as in the range of 6.5 to 8.5;
   (d) separating the rehydrated seaweed of step (c) from the solution;
   (e) drying the product of step (d), and
   (f) optionally milling the dried product of step (e) to form the food ingredient.

All publications mentioned in the above specification are herein incorporated by reference. Various modifications and variations of the described methods and composition of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry applied in food industry or related fields are intended to be within the scope of the following claims.

1. A food ingredient obtained from seaweed of the class of Rhodophyta wherein:
(a) the food ingredient contains mu carrageenan in an amount of at least 4 wt. % based on the total weight of the food ingredient; and
(b) the weight average molecular weight of carrageenan present in the food ingredient is at least 700.

2. A food ingredient according to claim 1 wherein the seaweed comprises at least one seaweed genera selected from Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus.

3. A food ingredient according to claim 1 wherein the seaweed comprises the genus Eucheuma or Kappaphycus.

4. A food ingredient according to claim 1 wherein the seaweed comprises at least one species selected from Euchema striatum/Kappaphycus striatus and Euchema alvarezii/Kappaphycus alvarezii.

5. A food ingredient according to claim 1 wherein the food ingredient contains mu carrageenan in an amount of at least 4 wt. % based on the total weight of the food ingredient.

6. A food ingredient according to claim 1 wherein the food ingredient contains mu carrageenan in an amount of from 6 to 20 wt. % based on the total weight of the food ingredient.

7. A food ingredient according to claim 1 wherein the weight average molecular weight of carrageenan present in the food ingredient is at least 1200 kDa.

8. A food ingredient according to claim 1 wherein the food ingredient contains acid insoluble matter in an amount of from 3 to 20 wt. % based on the total weight of the food ingredient.

9. A food ingredient according to claim 1 wherein the food ingredient contains acid insoluble matter in an amount of from 5 to 15 wt. % based on the total weight of the food ingredient.

10. A composition comprising:
(i) a food ingredient as defined in claim 1; and
(ii) (a) a protein; or
(b) a polysaccharide selected from galactomannans, glucomannans, and mixtures thereof.

11. A composition according to claim 10 wherein the protein is selected from soy protein, pea protein, and mixtures thereof.

12. A foodstuff containing a food ingredient as defined in claim 1.

13. A foodstuff according to claim 12 wherein the food ingredient is present in an amount of no greater than 7 wt. % based on the total weight of the foodstuff.

14. A foodstuff according to claim 12 wherein:
the foodstuff is a dairy foodstuff, and
the food ingredient is present in the foodstuff in an amount of in the range of 0.02 % wt. to 1 wt. % based on the total weight of the foodstuff.

15. A method for improving the stability of a foodstuff, wherein the method comprises adding the food ingredient defined in claim 1 to the foodstuff.

16. A method for for reducing sedimentation in a beverage, wherein the method comprises adding the food ingredient defined in claim 1 into the beverage.
